(19) United States
(12) Patent Application Publication
(10) Pub. No.: US 2006/0194871 A1
(43) Pub. Date: Aug. 31, 2006

(54) Heterocyclic mchr1 antagoists

(76) Inventors: Kevin K Barvian, Durham, NC (US); Andrew J Carpenter, Durham, NC (US); Joel P Cooper, Durham, NC (US); Paul L Feldman, Durham, NC (US); Dulce M Garrido, Durham, NC (US); Yu C Guo, Durham, NC (US); Anthony L Handlon, Durham, NC (US); Donald L Hertzog, Durham, NC (US); Clifton E Hyman, Durham, NC (US); Andrew J Peat, Durham, NC (US); Gregory E Peckham, Durham, NC (US); Jason D Speake, Durham, NC (US); William R Swain, Durham, NC (US); Francis X Tavares, Durham, NC (US); Huiqiang J Zhou, Durham, NC (US)

Correspondence Address: GLAXOSMITHKLINE;CORPORATE INTELLECTUAL PROPERTY, MAI B475, FIVE MOORE DR., PO BOX 13398, RESEARCH TRIANGLE PARK, NC, 27709-3398, US

(21) Appl. No.: 10/552,232

(22) PCT Filed: Apr. 6, 2004
(86) PCT No.: PCT/US04/10518

§ 371 Date: Oct. 7, 2005

Related U.S. Application Data
(60) Provisional application No. 60/462,292, filed on Apr. 11, 2003.

(51) Int. Cl. C07D 311/80; A61K 31/353

(52) U.S. Cl. 514/455; 549/387; 549/390

(57) ABSTRACT

This invention relates to novel heterocycles which are antagonists at the melanin-concentrating hormone receptor 1 (MCHR1), also referred to as 11 CBy, to pharmaceutical compositions containing them, to processes for their preparation, and to their use in medicines. Compounds of the invention have the formula:

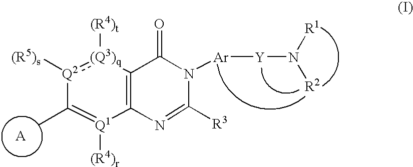

This invention relates to novel heterocycles which are antagonists at the melanin-concentrating hormone receptor 1 (MCHR1), also referred to as 11CBy, to pharmaceutical compositions containing them, to processes for their preparation, and to their use in therapy.

### BACKGROUND OF THE INVENTION

Obesity is a medical condition that is reaching epidemic proportions among humans in a number of countries throughout the world. It is a condition that is also associated with or induces other diseases or conditions that disrupt life activities and lifestyles. Obesity is recognized as a serious risk factor for other diseases and conditions such as diabetes, hypertension, and arteriosclerosis. It is also known that increased body weight due to obesity can place a burden on joints, such as knee joints, causing arthritis, pain, and stiffness.

Because overeating and obesity have become such a problem in the general population, many individuals are now interested in losing weight, reducing weight, and/or maintaining a healthy body weight and desirable lifestyle.

WO01/21577 (Takeda) relates to a compound of the formula 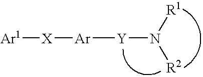
wherein Ar¹ is a cyclic group which may have substituents, X is a spacer having a main chain of 1 to 6 atoms, Y is a bond or a spacer having a main chain of 1 to 6 atoms, Ar is a monocyclic aromatic ring which may be condensed with a 4 to 8 membered non-aromatic ring, and may have further substituents; R¹ and R² are independently hydrogen or a hydrocarbon group which may have substituents; R¹ and R² together with the adjacent nitrogen atom may form a nitrogen containing hetero ring which may have substituents; R² may form a spiro ring together with Ar; or R² together with the adjacent nitrogen atom may form a nitrogen containing hetero ring which may have substituents; or a salt thereof; and which compounds are antagonists of a melanin-concentrating hormone. Such compounds are suggested as being useful for preventing or treating obesity.

WO 01/82925A1 (Takeda) relates to a compound of the formula 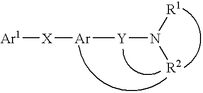
wherein Ar¹ is an optionally substituted cyclic group;
X and Y are the same or different spacers having from 1 to 6 atoms in the main chain;
Ar is an optionally substituted fused polycyclic aromatic ring;
R¹ and R² are the same or different hydrogen atoms or optionally substituted hydrocarbon groups, or R¹ and R² together with the adjacent nitrogen atoms may form an optionally substituted nitrogenous heterocycle, R² together with the adjacent nitrogen atom and Y may form an optionally substituted nitrogenous heterocycle, or R² together with the adjacent nitrogen atom, Y, and Ar may form an optionally substituted nitrogenous heterocycle or salts thereof.

WO 01/21577A2 (Takeda) relates to aromatic compounds of the formula 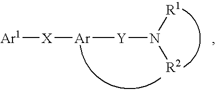
or a salt thereof, which is useful as an agent for preventing or treating obesity.

P32897WO1 (GlaxoSmithKline) relates to compounds of the formula

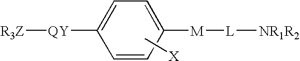

or a salt thereof, wherein M is a group selected from O, S, CO, NH or CH₂, L is a 2 or 3 membered alkylene chain, and the chain -M-L may be optionally substituted by one or more groups selected from methyl, ethyl, hydroxy or alkoxy and or which chain may contain a —C═C— double bond; R₁ and R₂ each independently represent hydrogen, C₁₋₆ straight or branched alkyl which may be optionally substituted by phenyl, or C₃₋₆ cycloalkyl optionally substituted by one or more C₁₋₆ alkyl groups; or R₁ and R₂ together with the nitrogen atom to which they are attached form a 4-8 membered heterocyclic ring or a 7-10 membered bridged heterocyclic ring, which rings may be optionally substituted by a phenyl group or up to 4 C₁₋₃ alkyl groups; or R₁ or R₂ may be linked to the group L or be linked as part of the substituted X on the phenyl ring to form a cyclic group; the group X may be linked to the group L to form a cyclic group which may contain an additional oxygen, a sulphur or nitrogen atom, alternatively or additionally there may be one or more substituents X selected from hydroxy, C₁₋₂ alkyl, C₁₋₂ alkoxy, halogen, C₂₋₃ alkenyl, benzyl, CRₐNOR_{b} wherein Rₐ and R_{b} are independently hydrogen or methyl, methoxy-methyl, methoxymethoxy or methoxyethoxy; QY is a bicyclic fused heterocyclic ring wherein Y is one ring of a bicyclic fused heterocyclic group and which is linked via nitrogen atom therein to the phenyl ring, and substituted on the second ring Q by the group ZR₃; Z is a bond or a group selected from NH, NCH₃O, S or CH₂; R₃ is a group selected from aryl, 2-alkenyl, cycloalkyl or 2-cycloalkenyl and which R₃ group may be optionally substituted by one or more C₁₋₃ alkyl, halo, amino, alkylamino, dialkylamino, hydroxy, C₁₋₃ alkoxy, cyano, trifluoromethyl or methylthio groups, processes for their preparation, pharmaceutical compositions containing them and to their use in medicine.

P32897WO2 (GlaxoSmithKline) relates to a compound of the formula comprising: 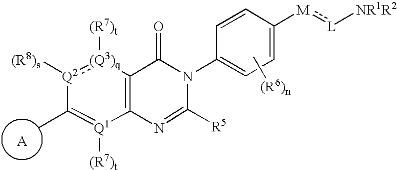
a pharmaceutically acceptable salt or solvate thereof, formulations, processes of preparing, and methods of administering to mammals are provided.

Aventis WO 03/015769A1 relates to aminoalkyl-substituted aromatic compounds of the formula below, their physiologically funcitonal derivatives and salts, as well as a method for a the production thereof. Said compounds can be suitably used as anorectic drugs.

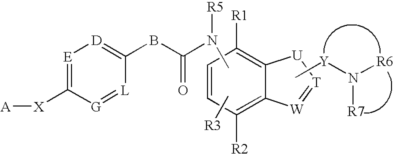

In particular, it is known that melanin-concentrating hormone (“MCH”) originates in the hypothalamus and has orexigenic action (see Nature, Vol. 396, p. 670, (1998), for example). There is an on-going need for the development of a melanin-concentrating hormone antagonist useful in the treatment of obesity and other associated or related diseases and conditions.

Accordingly, we have now found a novel group of heterocycles that exhibit a useful profile of activity as antagonists of the melanin-concentrating hormone receptor (MCHR1) disclosed in Nature, Vol. 400, p. 261-265 (1999).

### SUMMARY OF THE INVENTION

The present invention provides a compound of formula (I) comprising: 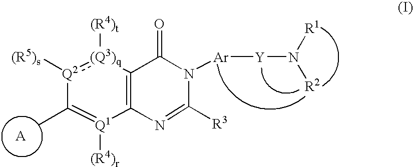
a pharmaceutically acceptable salt, solvate, or phyisiologically functional derivative thereof, wherein:
\textcircled{A} is aryl or heteroaryl, optionally substituted one to four times by a least one substiutent selected from the group consisting of C₁₋₆ straight or branched alkyl, alkenyl, halo, amino, alkylamino, dialkylamino, hydroxy, C₁₋₆ alkoxy, cyano, nitro, and alkylthio groups;

the dashed line connecting Q² to Q³ represents an optional bond;

q, r, s, and t are each independently 0 or 1;

when q is 1, the bond between Q² and Q³ is a double bond;

Q¹ and Q³ are each independently C or N;

when q is 0 then Q² is N, S, or O;

when q is 1, then Q² is C or N; when q is 1 and Q² is N, then s is 0;

when Q² is S or O, s is 0;

when Q¹ is N, r is 0;

when Q³ is N, t is 0;

R³ is selected from the group consisting of hydrogen, amino, C₁₋₆ straight or branched alkyl, C₃₋₆ cycloalkyl, and C₁₋₃ alkylthio;

when Q¹ or Q³ is C, then each corresponding R⁴ is independently selected from the group consisting of hydrogen, C₁₋₆ straight or branched alkyl, C₃₋₆ cycloalkyl, C₁₋₆ alkoxy, amino, alkylamino, dialkylamino, hydroxy, cyano, alkylthio, and halo;

when q is 1 and Q² is C or when q is 0 and Q² is N, then R⁵ is selected from hydrogen, C₁₋₆ straight or branched alkyl, C₃₋₆ cycloalkyl, C₁₋₆ alkoxy, amino, alkylamino, dialkylamino, hydroxy, cyano, alkylthio, and halo;

Ar is a fused bicyclic ring optionally substituted one to four times by at least one substituent selected from the group consisting of C₁₋₆ straight or branched alkyl, alkenyl, halo, amino, alkylamino, dialkylamino, hydroxy, C₁₋₆ alkoxy, cyano, and alkylthio groups;

Y is a bond or a C₁₋₆ alkylene, optionally substituted;

(i) R¹ and R² each independently are selected from the group consisting of hydrogen, C₁₋₆ straight or branched alkyl, C₃₋₆ cycloalkyl, and a 5- or 6-membered heterocycle wherein said alkyl, said cycloalkyl, and said heterocycle are optionally substituted one to four times by at least one substituent selected from the group consisting of phenyl, C₁₋₃ alkyl, amino, C₁₋₆ alkylamino, C₁₋₆ dialkylamino, hydroxy, oxo (i.e., ═O), alkoxy and halo;

or (ii) R¹ and R² may be selected from the group consisting of aryl and a 5- or 6-membered heteroaryl containing 1, 2, or 3 heteroatoms selected from N, O, and S, wherein said aryl and said heteroaryl are optionally substituted 1, 2, or 3 times with a substituent selected from halo, C₁₋₆ straight or branched alkyl, C₃₋₆ cycloalkyl, C₁₋₆ alkenyl, C₃₋₆ cycloalkenyl, hydroxy, C₁₋₆ alkoxy, amino, C₁₋₆ alkylamino, C₁₋₆ dialkylamino, C₁₋₆ alkylthio, C₁₋₆ alkylsulfinyl, and phenyl;

or (iii) R¹ and R² together with the nitrogen atom to which they are bonded form a 4-8 membered heterocyclic ring or a 7-11 membered bicyclic heterocyclic ring, each of said 4-8 membered heterocyclic ring and said 7-11 membered bicyclic heterocyclic ring contain 1, 2 or 3 heteroatoms selected from the group consisting of N, O, and S, and wherein either said heterocyclic ring or said bicyclic heterocyclic ring may be optionally substituted one to four times by at least one substituent selected from the group consisting of phenyl, C₁₋₃ alkyl, hydroxy, C₁₋₃ alkoxy, oxo (i.e., ═O), amino, C₁₋₆ alkylamino, C₁₋₆ dialkylamino, and halo;

or (iv) R² together with the adjacent nitrogen atom and Y may form an optionally substitued nitrogen-containing heterocycle, or R² together with the adjacent nitorgen atom, Y, and Ar may form an optionally substitued nitrogen-containing heterocycle or salt thereof, and wherein said heterocycles are optionally substituted one to four times by at least one substituent selected from the group consisting of phenyl, C₁₋₃ alkyl, hydroxy, C₁₋₃ alkoxy, oxo (i.e., ═O), amino, C₁₋₆ alkylamino, C₁₋₆ dialkylamino, and halo;

In another aspect of the invention, there is provided a pharmaceutical composition for use in the treatment, prophylaxis or both of one or more conditions or indications set forth herein comprising a compound of formula (I), or a physiologically acceptable salt, solvate, or physiologically functional derivative thereof, and a pharmaceutically acceptable carrier or excipient. There is also provided a method of treatment comprising the administration of the above-identified compound of formula (I) to a mammal such as a human, as well as, the use of said compound in the manufacture of a medicine for treating the conditions of obesity, diabetes, depression, and/or anxiety in a mammal (e.g., a human).

In a further embodiment of the invention, there are provided processes for the preparation a compound of formula (I).

### DETAILED DESCRIPTION OF THE INVENTION

As used herein, “a compound of the invention” or “a compound of formula (I)” means a compound of formula (I) or a pharmaceutically acceptable salt, solvate, of physiologically functional derivative (such as, e.g. a prodrug), thereof.

As used herein, unless otherwise specified, the term “alkyl” and “alkylene” refer to straight or branched hydrocarbon chains containing 1 to 6 carbon atoms. Examples of “alkyl” as used herein include, but are not limited to, methyl, ethyl, n-propyl, n-butyl, n-pentyl, isobutyl, isopropyl, tert-butyl, and hexyl. Examples of “alkylene” as used herein include, but are not limited to, methylene, ethylene, propylene, butylene, and isobutylene. “Alkyl” also includes substituted alkyl. “Alkylene” also includes substituted alkylene. The alkyl and alkylene groups may optionally be substituted with at least one substituent selected from the group consisting of hydroxy, alkoxy, halo, amino, alkylamino, dialkylamino, thio, oxo, aryl, and cyano. Halo, alkoxy, and hydroxy are particularly preferred.

As used herein, unless otherwise specified, the term “cycloalkyl” refers to a non-aromatic carbocyclic ring having from 3 to 8 carbon atoms (unless otherwise specified) and no carbon-carbon double bonds. “Cycloalkyl” includes by way of example cyclopropyl, cyclobutyl, cyclopentyl, cyclohexyl, cycloheptyl, and cyclooctyl. “Cycloalkyl” also includes substituted cycloalkyl. The cycloalkyl may be optionally substituted with at least one substituent selected from the group consisting of hydroxy, cyano, halo, alkoxy, amino, alkylamino, dialkylamino, and alkyl. Halo, hydroxy, and alkoxy are preferred.

As used herein, unless otherwise specified, the term “alkenyl” refers to straight or branched hydrocarbon chains containing 2 to 8 carbon atoms and at least one and up to three carbon-carbon double bonds. Examples of “alkenyl” as used herein include, but are not limited to, ethenyl and propenyl. “Alkenyl” also includes substituted alkenyl. The alkenyl group may be optionally substituted with at least one substituent selected from the group consisting of alkyl, amino, alkylamino, dialkylamino, halo, hydroxy, alkoxy, and cyano. Halo, hydroxy, and alkoxy are preferred.

As used herein, unless otherwise specified, the term “cycloalkenyl” refers to a non-aromatic carbocyclic ring having from 3 to 8 carbon atoms (unless otherwise specified) and up to 3 carbon-carbon double bonds. “Cycloalkenyl” includes by way of example, cyclobutenyl, cyclopentenyl, and cyclohexenyl. “Cycloalkenyl” also includes substituted cycloalkenyl. The ring may be optionally substituted with at least one substituent selected from the group consisting of cyano, halo, hydroxy, —NH₂, —N₃, —CN, —O—C₁₋₃ alkyl, —NH(C₁₋₃ alkyl), —N(C₁₋₃ alkyl)₂, and —C₁₋₃ alkyl (including haloalkyl).

As used herein, the terms “halo” or “halogen” refer to fluorine, chlorine, bromine, and iodine. Preferred among these are chlorine (or “chloro”) and fluorine (or “fluoro”).

Unless otherwise specified, the term, “aryl” (as well as “aromatic”) refers to monocyclic carbocyclic groups and fused bicyclic carbocylic groups having from 6 to 12 carbon atoms and having at least one aromatic ring. Examples of particular aryl groups include, but are not limited to, phenyl and naphthyl. “Aryl” also includes substituted aryl, especially substituted phenyl. An aryl ring may be optionally substituted with at least one substituent selected from the group consisting of halo, alkyl (including haloalkyl), alkenyl, cycloalkyl, cycloalkenyl, alkoxy, amino, hydroxy, hydroxyalkyl, aminoalkyl, carboxy, carboxamide, sulfonamide, heteroaryl (abbreviated as “Het”), amidine, cyano, nitro, and azido. Preferred aryl groups according to the invention include, but are not limited to, phenyl and substituted phenyl. Preferred substituted phenyl is a phenyl containing one or more halo groups, particularly chloro and fluoro groups.

The terms “heterocycle” and “heterocyclic” refer to a ring system composed of C and at least one other atom selected from the group consisting of N, O, and S. Heterocycles may or may not be heteroaromatic as defined below. In other words, heteroaromatics are heterocycles, but all heterocycles are not heteroaromatic.

The term “heteroaryl” and “heteroaromatic” refer to a monocyclic or bicylic aromatic ring system composed of C and at least one other atom selected from the group consisting of N, O, and S.

The terms “members” (and variants thereof, e.g., “membered”) in the context of heterocyclic, heteroaryl, and aryl groups refers to the total atoms, carbon and heteroatoms (N, O, and/or S) which form the ring. Thus, an example of a 6-membered heterocyclic ring is piperidine, an example of a 6-membered heteroaryl ring is pyridine, and an example of a 6-membered aryl ring is benzene.

As used herein, the term “optionally” means that the subsequently described event(s) may or may not occur, and includes both event(s) that occur and events that do not occur.

Formula (I) of the invention is set forth in detail as follows.
\textcircled{A} is aryl or heteroaryl, optionally substituted one to four times with at least one substituent selected from the group consisting of C₁₋₆ straight or branched alkyl, alkenyl, halo, amino, alkylamino, dialkylamino, hydroxy, C₁₋₆ alkoxy, cyano, nitro, and alkylthio groups. Preferred among these substituted groups are halo, C₁₋₃ alkyl, and C₁₋₃ alkoxy. Most preferred are fluoro, chloro, and methoxy. In a preferred embodiment said \textcircled{A} is substituted with a halo group, q is 0, Q¹ is carbon, Q² is sulfur, and R⁴ is hydrogen or halo. For example, \textcircled{A} is 4-chlorophenyl and R³ and R⁴ are each hydrogen.

In the formula, the dashed line connecting Q² to Q³ represents an optional bond such that the bond between Q² and Q³ are connected by a double bond; and q, r, s, and t are each independently 0 or 1.

In formula (I), q is 0 or 1. When q is 1 the bond between Q² and Q³ in formula (I) is a double bond. When q is 0 there is no Q³ group. When q is 0 then Q² is N, S, or O. And when q is 1, Q² is C or N. When q is 1 and Q² is N, then s is 0 and there is no R⁵ substituent.

Q¹ and Q³ are each independently carbon (C) or nitrogen (N). In one embodiment, Q¹, Q², and Q³ are each carbon and q, r, s, and t are 1. In another embodiment, Q¹ is carbon, Q² is sulfur, q and s are 0, and r is 1.

In the formula, r and t are each independently 0 or 1. When r and t are each independently 0, then there is no R⁴ substituent. When r and t are each independently 1, Q¹ and Q³ are each independently bonded by the group R⁴. Each R⁴ is the same or different and is independently selected from the group consisting of hydrogen, C₁₋₆ straight or branched alkyl, C₃₋₆ cycloalkyl, C₁₋₆ alkoxy, amino, alkylamino, dialkylamio, hydroxy, cyano, alkylthio, and halo.

In formula (I), s is 0 or 1. When Q² is S or O, then s is 0 and there is no R⁵ group. When Q² is C, then s is 1 and R⁵ is selected from the group consisting of hydrogen, C₁₋₆ straight or branched alkyl, C₃₋₆ cycloalkyl, C₁₋₆ alkoxy, amino, alkylamino, dialkylamino, hydroxy, cyano, alkylthio, and halo. When Q² is C, preferably R⁵ is hydrogen or a C₁₋₃ alkyl; most preferably R⁵ is hydrogen or methyl.

In formula (I), R³ is selected from the group consisting of hydrogen, amino, C₁₋₆ straight or branched alkyl, and C₃₋₆ cycloalkyl. Preferably, R³ is hydrogen or a C₁₋₃ alkyl; most preferably R³is hydrogen or methyl.

When either or both Q¹ and Q³ are C, then R⁴is selected from the group consisting of hydrogen, C₁₋₆ straight or branched alkyl, C₃₋₆ cycloalkyl, C₁₋₆ alkoxy, amino, alkylamino, dialkylamino, hydroxy, cyano, alkylthio, and halo. Preferably, when either or both Q¹ and Q³ are C, R⁴is hydrogen or C₁₋₃ alkyl; most preferably R⁴is hydrogen or methyl.

When Q² is N, and s is 1, R⁵ is selected from the group consisting of hydrogen, C₁₋₆ straight or branched alkyl, C₃₋₆ cycloalkyl, C₁₋₆ alkoxy, amino, alkylamino, dialkyl amino, hydroxy, cyano, alkylthio, and halo. When Q² is N, and s is 1, preferably R⁵ is hydrogen or a C₁₋₃ alkyl; most preferably R⁵ is hydrogen or methyl.

In the formula (I), Ar is an optionally substituted fused bicyclic ring having 9 to 14 members, optionally substituted one to four times by at least one substituent selected from the group consisting of C₁₋₆ straight or branched alkyl, alkenyl, halo, amino, alkylamino, dialkylamino, hydroxy, C₁₋₆ alkoxy, cyano, and alkylthio groups. That is, Ar can be a fused bicyclic ring having: (i) two aromatic rings fused together, (ii) an aromatic ring and a heteroaromatic ring fused together, (iii) two heteroaromatic rings fused together, (iv) an aromatic ring fused to a heterocyclic ring, or (v) having an aromatic ring fused to a carbocyclic ring. Preferably, Ar is selected from the group consisting of quinoline, naphthalene, benzimidazole, indole, benzothiophene, benzofuran, and benzothiazole. When Ar is a ten-membered bicyclic aromatic or ten-membered bicyclic heteroaromatic ring, then preferably Ar is quinoline or naphthalene. When Ar is a 9-membered fused bicyclic heteroaromatic ring, then preferably Ar is benzimidazole, indole, benzothiophene, benzofuran, or benzothiazole.

In the formula (I), Y is a bond or a C₁₋₆ alkylene, optionally substituted as defined herein. When Ar is a ten-membered polycyclic aromatic or ten-membered polycyclic heteroaromatic ring, then preferably Y is a C₁₋₃ alkylene, optionally substituted; most preferably Y is methylene (—CH₂—), optionally substituted. When Ar is a 9-membered fused polycyclic heteroaromatic ring, then preferably Y is a bond or a C₁₋₃ alkylene, optionally substituted; most preferably Y is a bond.

In (i), R¹ and R² of formula (I) are each independently selected from the group consisting of hydrogen, C₁₋₆ straight or branched alkyl, C₃₋₆ cycloalkyl, phenyl, and 5- or 6-membered heterocycle, wherein said alkyl, said cycloalkyl, and said heterocycle are optionally substituted one to four times by at least one substituent selected from the group consisting of phenyl, C₁₋₃ alkyl, amino, C₁₋₆ alkylamino, C₁₋₆ dialkylamino, hydroxy, oxo, alkoxy, and halo. Preferably, R¹ and R² are each independently selected from the group consisting of hydrogen, C₁₋₆ straight or branched alkyl, and C₃₋₆ cycloalkyl. Most preferably, R¹ and R² are each independently selected from the group consisting of hydrogen, C₁₋₃ alkyl, and C₃₋₆ cycloalkyl.

Or, in (ii), R¹ and R² are selected from the group consisting of aryl and a 5- or 6-membered heteroaryl containing 1, 2, or 3 heteroatoms selected from N, O, and S, wherein said aryl and said heteroaryl are optionally substituted 1, 2, or 3 times with at least one substituent selected from the group consisting of halo, C₁₋₆ straight or branched alkyl, C₃₋₆ cycloalkyl, C₁₋₆ alkenyl, C₃₋₆ cycloalkenyl, hydroxy, C₁₋₆ alkoxy, amino, C₁₋₆ alkylamino, C₁₋₆ dialkylamino, C₁₋₆ alkylthio, C₁₋₆ alkylsulfinyl, and phenyl. Preferably, when either R¹ or R² is aryl or heteroaryl, the other remaining R¹ or R² is a hydrogen, a C₁₋₆ alkyl, or a C₃₋₆ cycloalkyl.

Additionally, in (iii), R¹ and R² together with the nitrogen atom to which they are bonded can form a 4-8 membered heterocyclic ring or a 7-11 membered bicyclic heterocyclic ring. The 4-8 membered heterocyclic ring and/or the 7-11 membered bicyclic heterocyclic ring may contain 1, 2, or 3 heteroatoms selected from the group consisting of N, O, and S. And either the heterocyclic ring or the bicyclic heterocyclic ring may be optionally substituted one to four times by at least one substituent selected from the group consisting of phenyl, C₁₋₃ alkyl, hydroxy, C₁₋₃ alkoxy, amino, C₁₋₆ alkylamino, C₁₋₆ dialkylamino, oxo, and halo. Here neither group R¹ or R² is linked to M or L. Preferably, R¹ and R² together form a 5- or 6-membered heterocyclic ring or an 8- to 11-membered bicylic heterocyclic ring, having 1 or 2 heteroatoms selected from the group N, O, and S wherein said heterocyclic ring and said bicyclic heterocyclic ring may be optionally substituted up to two times with a substituent selected from the group consisting of oxo and halo.

Also additionally, in (iv), R² together with the adjacent nitrogen atom and Y may form an optionally substituted nitrogen-containing heterocycle, or R² together with the adjacent nitrogen atom, Y, and Ar may form an optionally substituted nitrogen-containing heterocycle or salt thereof. The said nitrogen-containing heterocycles are optionally substituted one to four times by at least one substituent selected from the group consisting of phenyl, C₁₋₃ alkyl, hydroxy, C₁₋₃ alkoxy, oxo (i.e., ═O), amino, C₁₋₆alkylamino, C₁₋₆ dialkylamino, and halo. Preferably, R² together with the adjacent nitrogen atom and Y form a 3-7 membered ring when Y is a C₁₋₆ alkyl group. Most preferably a 5-7 membered ring is formed. The 5-7 membered ring is optionally substituted by at least one substitutent selected from the group consisting of phenyl, one to four C₁₋₃ alkyl, hydroxy, alkoxy, oxo, amino, C₁₋₆ alkylamino, C₁₋₆ dialkylamino, or halo.

In one embodiment, when Ar is a 10-membered aromatic ring or a 10-membered heteroaromatic ring, the most preferred compounds according to this invention are selected from the group consisting of
6-(4-chlorophenyl)-3-{6-[(4-hydroxy-1-piperidinyl)methyl]-2-naphthalenyl}thieno[3,2-d]pyrimidin-4(3H)-one;
6-(4-chlorophenyl)-3-[6-(pyrrolidin-1-ylmethyl)-2-naphthyl]thieno[3,2-d]pyrimidin-4(3H)-one;
6-(4-chlorophenyl)-3-{2-[(4-methylpiperazin-1-yl)methyl]-1-benzothien-5-yl}thieno[3,2-d]pyrimidin-4(3H)-one;
6-(4-fluorophenyl)-3-[2-(piperidin-1-ylmethyl)quinolin-6-yl]thieno[3,2-d]pyrimidin-4(3H)-one;
6-(4-chlorophenyl)-3-{2-[(2-methyl-4,5-dihydro-1H-imidazol-1-yl)methyl]quinolin-6-yl}thieno[3,2-d]pyrimidin-4(3H)-one;
6-(4-chlorophenyl)-3-{2-[(2,2,6,6-tetramethylpiperidin-1-yl)methyl]quinolin-6-yl}thieno[3,2-d]pyrimidin-4(3H)-one;
6-phenyl-3-[2-(pyrrolidin-1-ylmethyl)quinolin-6-yl]thieno[3,2-d]pyrimidin-4(3H)-one;
6-phenyl-3-[2-(pyrrolidin-1-ylmethyl)quinolin-6-yl]thieno[3,2-d]pyrimidin-4(3H)-one.

In another embodiment, when Ar is a 9-membered heteroaromatic ring, the most preferred compound according to this invention is 6-(4-chlorophenyl)-3-[2-(dimethylamino)-1-methyl-1H-benzimidazol-6-yl]thieno[3,2-d]pyrimidin-4(3H)-one.

Certain compounds of formula (I) may exist in stereoisomeric forms (e.g., they may contain one or more asymmetric carbon atoms or may exhibit cis-trans isomerism). The individual stereoisomers (enantiomers and diastereomers) and mixtures of these are included within the scope of the present invention. The present invention also covers the individual isomers of the compounds represented by formula (I) as mixtures with isomers thereof in which one or more chiral centers are inverted. Certain compounds of formula (I) may be prepared as regioisomers. The present invention covers both the mixture of regioisomers as well as individual compounds. Likewise, it is understood that compounds of formula (I) may exist in tautomeric forms other than that shown in the formula and these are also included within the scope of the present invention.

It is to be understood that the present invention includes all combinations and subsets of the particular groups defined hereinabove. Specific compounds of formula (I) include but are not limited those set forth in Table I and/or those prepared examples herein.

TABLE I

| Example | | |
|---|---|---|
| No. | Structure | Name |
| 1 | 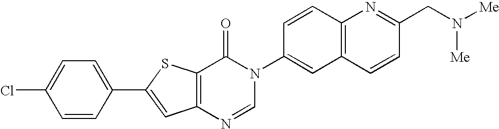 | 6-(4-chlorophenyl)-3-{2- [(dimethylamino)methyl]quinolin- 6-yl}thieno[3,2-d]pyrimidin-4(3H)- one |
| 2 | 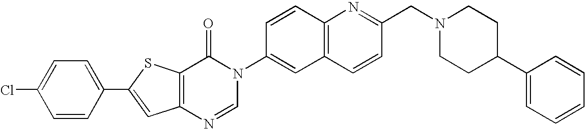 | 6-(4-chlorophenyl)-3-{2-[(4- phenylpiperidin-1- yl)methyl]quinolin-6-yl}thieno[3,2- d]pyrimidin-4(3H)-one |
| 3 | 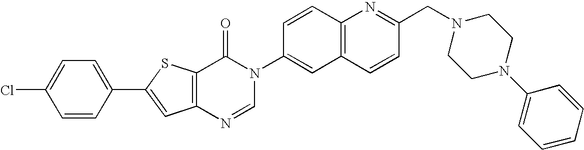 | 6-(4-chlorophenyl)-3-{2-[(4- phenylpiperazin-1- yl)methyl]quinolin-6-yl}thieno[3,2- d]pyrimidin-4(3H)-one |
| 4 | 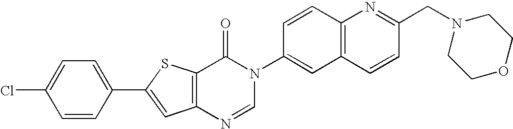 | 6-(4-chlorophenyl)-3-[2- (morpholin-4-ylmethyl)quinolin-6- yl]thieno[3,2-d]pyrimidin-4(3H)- one |
| 5 | 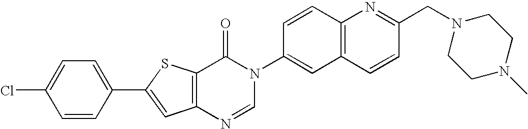 | 6-(4-chlorophenyl)-3-{2-[(4- methylpiperazin-1- yl)methyl]quinolin-6-yl}thieno[3,2- d]pyrimidin-4(3H)-one |
| 6 | 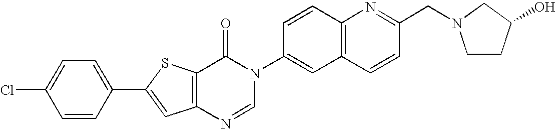 | 3-[2-hydroxymethyl)-6- quinolinyl]-6(4- methylphenyl)thieno[3,2- d]pyrimidin-4(3H)one |
| 7 | 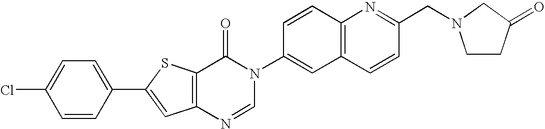 | 6-(4-chlorophenyl)-3-{2-[(3-oxo- 1-pyrrolidinyl)methyl]-6- quinolinyl}thieno[3,2-d]pyrimidin- 4(3H)-one |
| 8 | 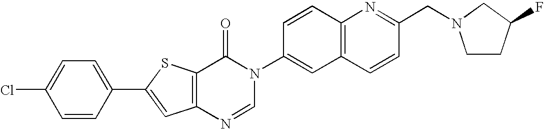 | 6-(4-chlorophenyl)-3-(2-{[(3S)-3- fluoropyrrolidinyl]methyl}-6- quinolinyl)thieno[3,2-d]pyrimidin- 4(3H)-one |
| 9 | 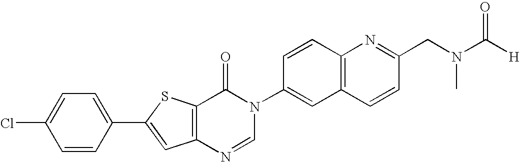 | [6-(6-(4-chlorophenyl)-4- oxothieno[3,2-d]pyrimidin-3(4H)- yl)-2-quinolinyl]methyl(methyl)- formamide |
| 10 | 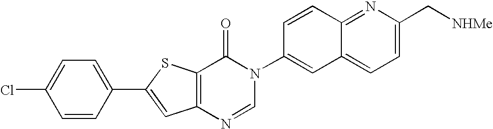 | 6-(4-chlorophenyl)-3-{2- [(methylamino)methyl]quinolin-6- yl}thieno[3,2-d]pyrimidin-4(3H)- one |
| 11 | 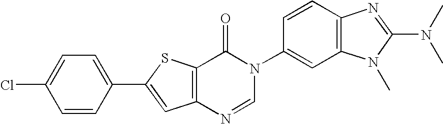 | 6-(4-chlorophenyl)-3-[2- (dimethylamino)-1-methyl-1H- benzimidazol-6-yl]thieno[3,2- d]pyrimidin-4(3H)-one |
| 12 | 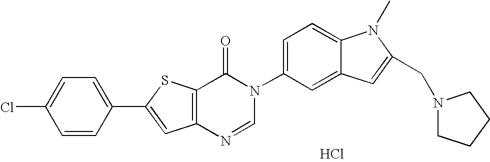 | 6-(4-chlorophenyl)-3-[1-methyl-2- (pyrrolidin-1-ylmethyl)-1H-indol- 5-yl]thieno[3,2-d]pyrimidin-4(3H)- one hydrochloride |
| 13 | 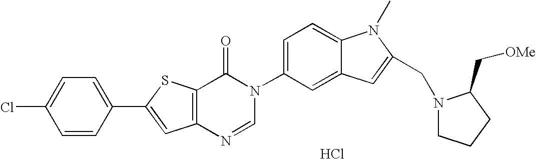 | 6-(4-chlorophenyl)-3-(2-{[(2R)-2- (methoxymethyl)pyrrolidin-1- yl]methyl}-1-methyl-1H-indol-5- yl)thieno[3,2-d]pyrimidin-4(3H)- one hydrochloride |
| 14 | 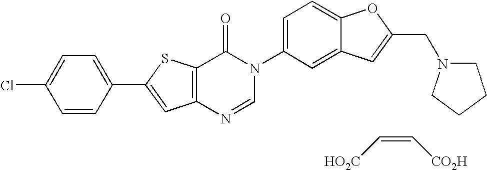 | 6-(4-methylphenyl)-3-[2- (pyrrolidin-1-ylmethyl)-1- benzofuran-5-yl]thieno[3,2- d]pyrimidin-4(3H)-one maleate salt |
| 15 | 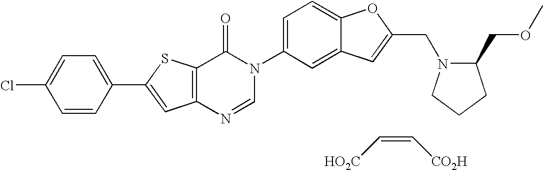 | 3-(2-{](2R)-2- (methoxymethyl)pyrrolidin-1- yl]methyl}-1-benzofuran-5-yl)-6- (4-methylphenyl)thieno[3,2- d]pyrimidin-4(3H)-one maleate salt |
| 16 | 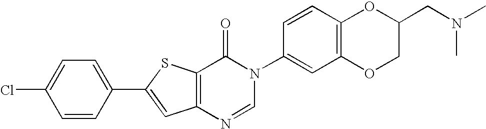 | 6-(4-chlorophenyl)-3-{2- [(dimethylamino)methyl]-2,3- dihydro-1,4-benzodioxin-6- yl}thieno[3,2-d]pyrimidin-4(3H)- one |
| 17 | 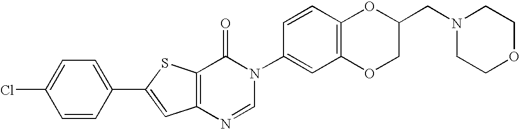 | 6-(4-chlorophenyl)-3-[2-(4- morpholinylmethyl)-2,3-dihydro- 1,4-benzodioxin-6-yl]thieno[3,2- d]pyrimidin-4(3H)-one |
| 18 | 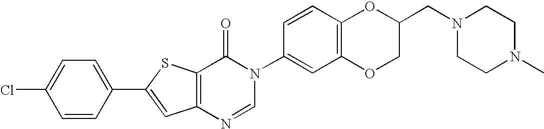 | 6-(4-chlorophenyl)-3-{2-[(4- methyl-1-piperazinyl)methyl]-2,3- dihydro-1,4-benzodioxin-6- yl}thieno[3,2-d]pyrimidin-4(3H)- one |
| 19 | 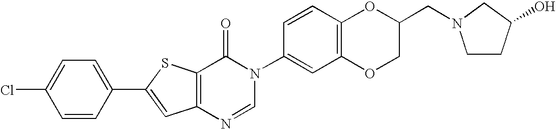 | 6-(4-chlorophenyl)-3-(2-{[(3R)-3- hydroxy-1-pyrrolidinyl]methyl}- 2,3-dihydro-1,4-benzodioxin-6- yl)thieno[3,2-d]pyrimidin-4(3H)- one |
| 20 | 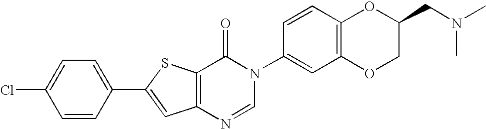 | 6-(4-chlorophenyl)-3-{(2S)-2- [(dimethylamino)methyl]-2,3- dihydro-1,4-benzodioxin-6- yl}thieno[3,2-d]pyrimidin-4(3H)- one |
| 21 | 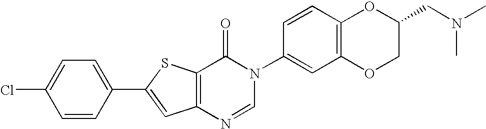 | 6-(4-chlorophenyl)-3-{(2R)-2- [(dimethylamino)methyl]-2,3- dihydro-1,4-benzodioxin-6- yl}thieno[3,2-d]pyrimidin-4(3H)- one |
| 22 | 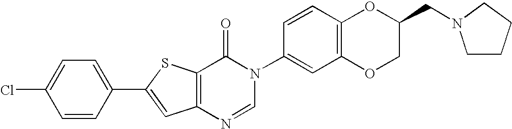 | 6-(4-chlorophenyl)-3-[(2S)-2-(1- pyrrolidinylmethyl)-2,3-dihydro- 1,4-benzodioxin-6-yl]thieno[3,2- d]pyrimidin-4(3H)-one |
| 23 | 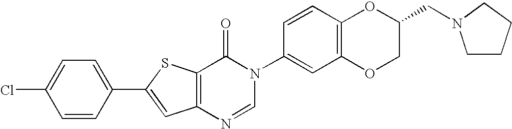 | 6-(4-chlorophenyl)-3-[(2R)-2-(1- pyrrolidinylmethyl)-2,3-dihydro- 1,4-benzodioxin-6-yl]thieno[3,2- d]pyrimidin-4(3H)-one |
| 24 | 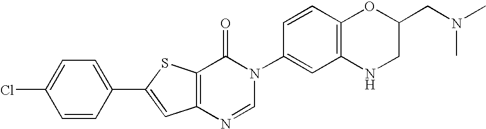 | 6-(4-chlorophenyl)-3-{2- [(dimethylamino)methyl]-3,4- dihydro-2H-1,4-benzoxazin-6- yl}thieno[3,2-d]pyrimidin-4(3H)- one |
| 25 | 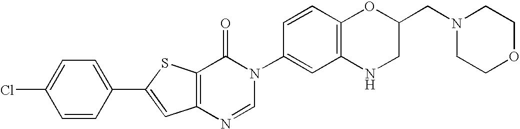 | 6-(4-chlorophenyl)-3-[2-(4- morpholinylmethyl)-3,4-dihydro- 2H-1,4-benzoxazin-6- yl]thieno[3,2-d]pyrimidin-4(3H)- one |
| 26 | 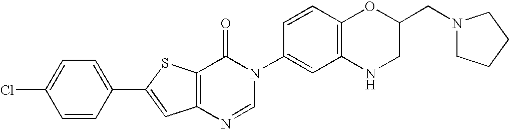 | 6-(4-chlorophenyl)-3-[2-(1- pyrrolidinylmethyl)-3,4-dihydro- 2H-1,4-benzoxazin-6- yl]thieno[3,2-d]pyrimidin-4(3H)- one |
| 27 | 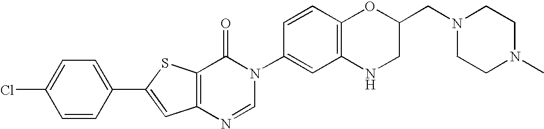 | 6-(4-chlorophenyl)-3-{2-[(4- methyl-1-piperazinyl)methyl]-3,4- dihydro-2H-1,4-benzoxazin-6- yl}thieno[3,2-d]pyrimidin-4(3H)- one |
| 28 | 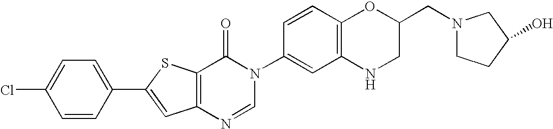 | 6-(4-chlorophenyl)-3-(2-{[(3R)-3- hydroxy-1-pyrrolidinyl]methyl}- 3,4-dihydro-2H-1,4-benzoxazin- 6-yl)thieno[3,2-d]pyrimidin-4(3H)- one |
| 29 | 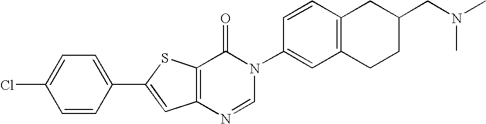 | 6-(4-chlorophenyl)-3-{6- [(dimethylamino)methyl]-5,6,7,8- tetrahydro-2- naphthalenyl}thieno[3,2- d]pyrimidin-4(3H)-one |
| 30 | 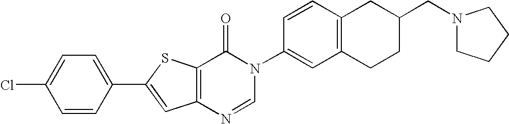 | 6-(4-chlorophenyl)-3-[6-(1- pyrrolidinylmethyl)-5,6,7,8- tetrahydro-2- naphthalenyl]thieno[3,2- d]pyrimidin-4(3H)-one |
| 31 | 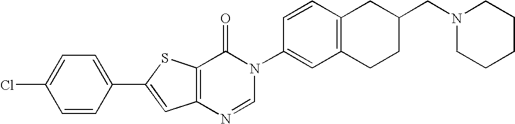 | 6-(40chlorophenyl)-3-[6-(1- piperidinylmethyl)-5,6,7,8- tetrahydro-2- naphthalenyl]thieno[3,2- d]pyrimidin-4(3H)-one |
| 32 | 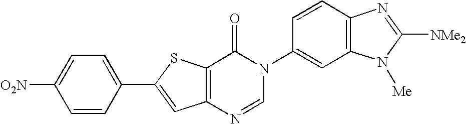 | 3-[2-(dimethylamino)-1-methyl- 1H-benzimidazol-6-yl]-6-(4- mitrophenyl)thieno[3,2- d]pyrimidin-4(3H)-one |
| 33 | 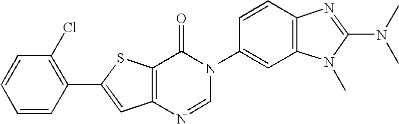 | 6-(2-chlorophenyl)-3-[2- (dimethylamino)-1-methyl-1H- benzimidazol-6-yl]thieno[3,2- d]pyrimidin-4(3H)-one |
| 34 | 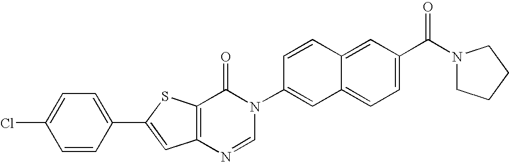 | 6-(4-chlorophenyl)-3-[6-(1- pyrrolidinylcarbonyl)-2- naphthalenyl]thieno[3,2- d]pyrimidin-4(3H)-one |
| 35 | 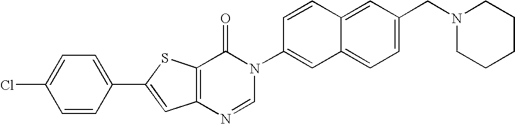 | 6-(4-chlorophenyl)-3-[6-(1- piperidinylmethyl)-2- naphthalenyl]thieno[3,2- d]pyrimidin-4(3H)-one |
| 36 | 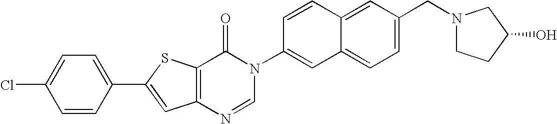 | 6-(4-chlorophenyl)-3-(6-{[(3R)-3- hydroxy-1-pyrrolidinyl]methyl}-2- naphthalenyl)thieno[3,2- d]pyrimidin-4(3H)-one |
| 37 | 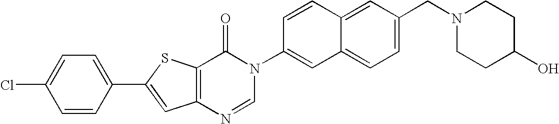 | 6-(4-chlorophenyl)-3-{6-[(4- hydroxy-1-piperidinyl)methyl]-2- naphthalenyl}thieno[3,2- d]pyrimidin-4(3H)-one |
| 38 | 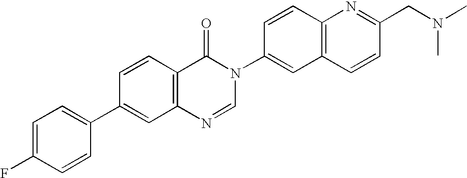 | 3-{2-[(dimethylamino)methyl]-6- quinolinyl}-7-(4-fluorophenyl)- 4(3H)-quinazolinone |
| 39 | 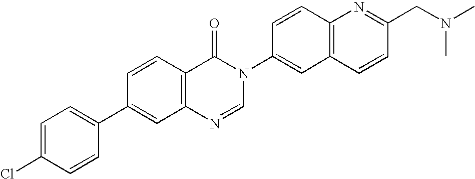 | 7-(4-chlorophenyl)-3-{2- [(dimethylamino)methyl]-6- quinolinyl}-4(3H)-quinazolinone |
| 40 | 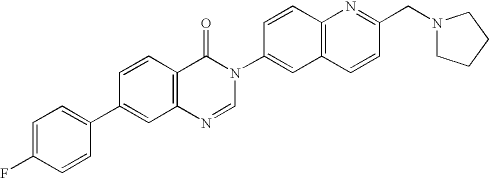 | 7-(4-fluorophenyl)-3-[2-(1- pyrrolidinylmethyl)-6-quinolinyl]- 4(3H)-quinazolinone |
| 41 | 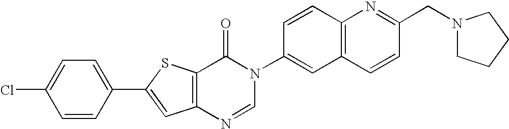 | 6-(4-chlorophenyl)-3-[2-(1- pyrrolidinylmethyl)-6- quinolinyl]thieno[3,2-d]pyrimidin- 4(3H)-one |
| 42 | 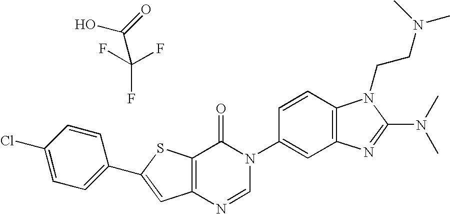 | 6-(4-chlorophenyl)-3-{2- (dimethylamino)-1-[2- (dimethylamino)ethyl]-1H- benzimidazol-5-yl}thieno[3,2- d]pyrimidin-4(3H)-one trifluoroacetate |
| 43 | 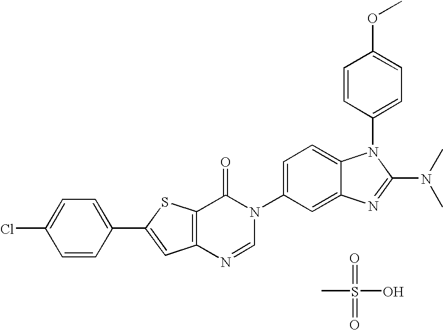 | 6-(4-chlorophenyl)-3-{2- (dimethylamino)-1-[4- (methyloxy)phenyl]-1H- benzimidazol-5-yl}thieno[3,2- d]pyrimidin-4(3H)-one methanesulfonate |
| 44 | 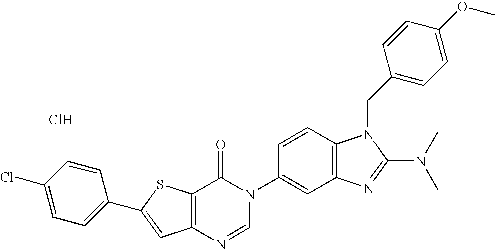 | 6-(4-chlorophenyl)-3-(2- (dimethylamino)-1-{[4- (methyloxy)phenyl]methyl}-1H- benzimidazol-5-yl)thieno[3,2- d]pyrimidin-4(3H)-one hydrochloride |
| 45 | 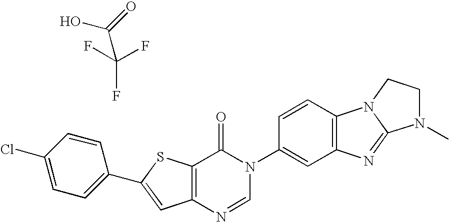 | 6-(4-chlorophenyl)-3-(1-methyl- 2,3-dihydro-1H-imidazo[1,2- a]benzimidazol-7-yl)thieno[3,2- d]pyrimidin-4(3H)-one trifluoroacetate |
| 46 | 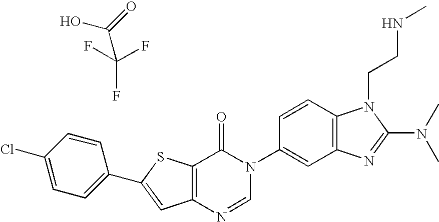 | 6-(4-chlorophenyl)-3-{2- (dimethylamino)-11[2- (methylamino)ethyl]-1H- benzimidazol-5-yl}thieno[3,2- d]pyrimidin-4(3H)-one trifluoroacetate |
| 47 | 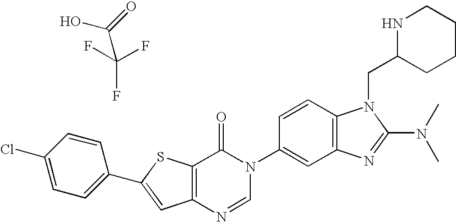 | 6-(4-chlorophenyl)-3-[2- (dimethylamino)-1-(2- piperidinylmethyl)-1H- benzimidazol-5-yl]thieno[3,2- d]pyrimidin-4(3H)-one trifluoroacetate |
| 48 | 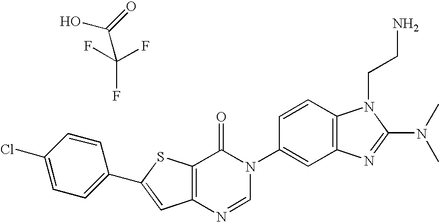 | 3-[1-(2-aminoethyl)-2- (dimethylamino)-1H- benzimidazol-5-yl]-6-(4- chlorophenyl)thieno[3,2- d]pyrimidin-4(3H)-one trifluoroacetate |
| 49 | 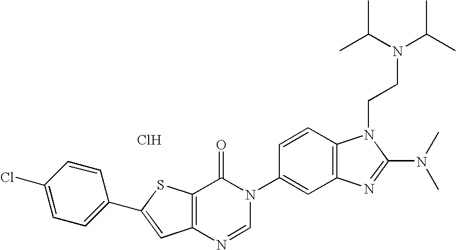 | 3-[1-{2-[bis(1- methylethyl)amino]ethyl}-2- (dimethylamino)-1H- benzimidazol-5-yl]-6-(4- chlorophenyl)thieno[3,2- d]pyrimidin-4(3H)-one hydrochloride |
| 50 | 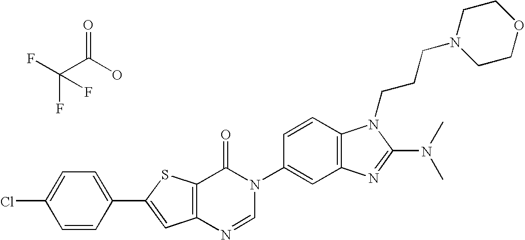 | 6-(4-chlorophenyl)-3-[2- (dimethylamino)-1-(3-morpholin- 4-ylpropyl)-1H-benzimidazol-5- yl]thieno[3,2-d]pyrimidin-4(3H)- one trifluoroacetate |
| 51 | 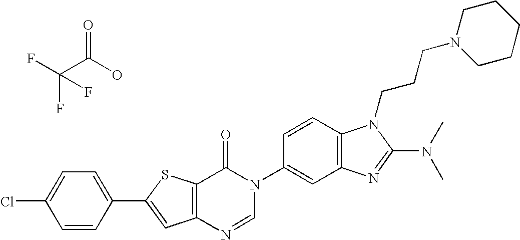 | 6-(4-chlorophenyl)-3-[2- (dimethylamino)-1-(3-piperidin-1- ylpropyl)-1H-benzimidazol-5- yl]thieno[3,2-d]pyrimidin-4(3H)- one trifluoroacetate |
| 52 | 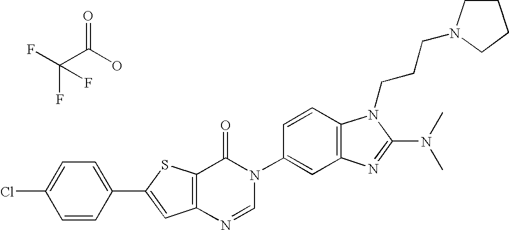 | 6-(4-chlorophenyl)-3-[2- (dimethylamino)-1-(3-pyrrolidin-1- ylpropyl)-1H-benzimidazol-5- yl]thieno[3,2-d]pyrimidin-4(3H)- one trifluoroacetate |
| 53 | 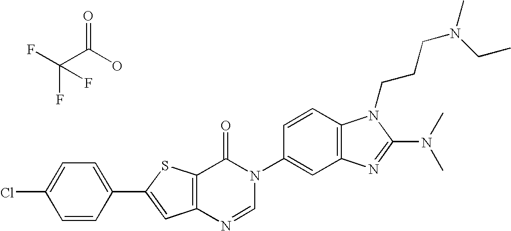 | 6-(4-chlorophenyl)-3-(2- (dimethylamino)-1-{3- [ethyl(methyl)amino]propyl}-1H- benzimidazol-5-yl)thieno[3,2- d]pyrimidin-4(3H)-one trifluoroacetate |
| 54 | 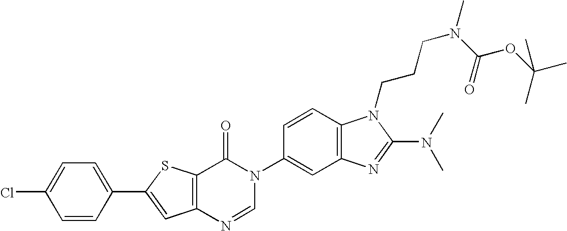 | tert-butyl {3-[5-[6-(4- chlorophenyl)-4-oxothieno[3,2- d]pyrimidin-3(4H)-yl]-2- (dimethylamino)-1H- benzimidazol-1- yl]propyl}methylcarbamate |
| 55 | 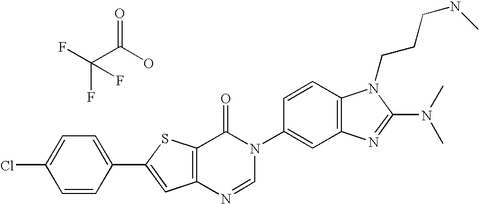 | 6-(4-chlorophenyl)-3-{2- (dimethylamino)-1-[3- (methylamino)propyl]-1H- benzimidazol-5-yl}thieno[3,2- d]pyrimidin-4(3H)-one trifluoroacetate |
| 56 | 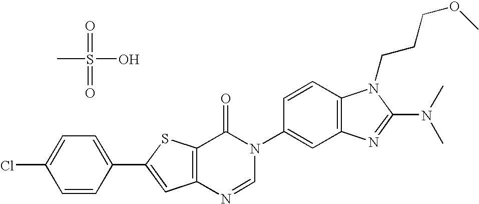 | 6-(4-chlorophenyl)-3-[2- (dimethylamino)-1-(3- methoxypropyl)-1H- benzimidazol-5-yl]thieno[3,2- d]pyrimidin-4(3H)-one methanesulfonate |
| 57 | 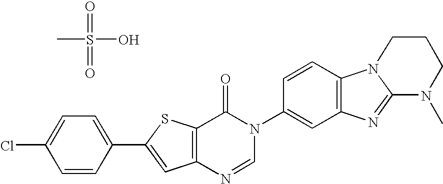 | 6-(4-chlorophenyl)-3-(1-methyl- 1,2,3,4-tetrahydropyrimido[1,2- a]benzimidazol-8-yl)thieno[3,2- d]pyrimidin-4(3H)-one methanesulfonate |
| 58 | 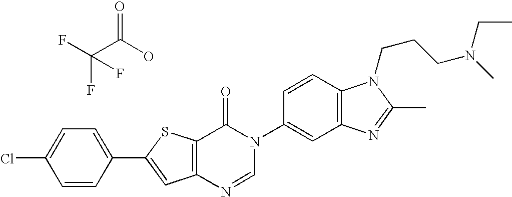 | 6-(4-chlorophenyl)-3-(1-{3- [ethyl(methyl)amino]propyl}-2- methyl-1H-benzimidazol-5- yl)thieno[3,2-d]pyrimidin-4(3H)- one trifluoroacetate |
| 59 | 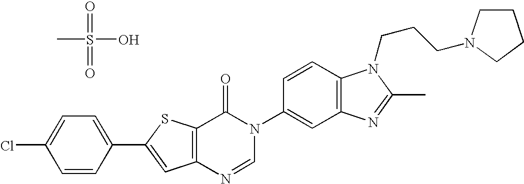 | 6-(4-chlorophenyl)-3-{2-methyl-1- [3-(1-pyrrolidinyl)propyl]-1H- benzimidazol-5-yl}thieno[3,2- d]pyrimidin-4(3H)-one methanesulfonate |
| 60 | 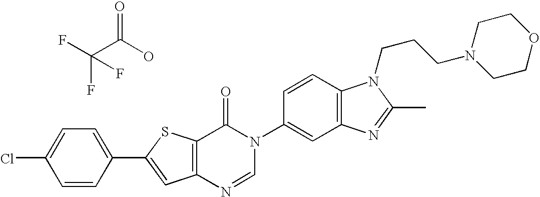 | 6-(4-chlorophenyl)-3-{2-methyl-1- [3-(4-morpholinyl)propyl]-1H- benzimidazol-5-yl}thieno[3,2- d]pyrimidin-4(3H)-one trifluoroacetate |
| 61 | 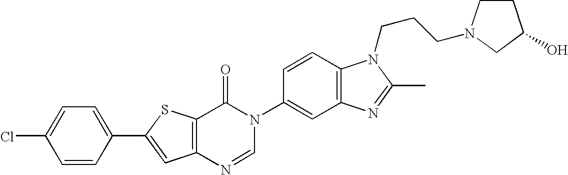 | 6-(4-chlorophenyl)-3-(1-{3-[(3S)- 3-hydroxy-1-pyrrolidinyl]propyl}- 2-methyl-1H-benzimidazol-5-yl)thieno[3,2-d]pyrimidin-4(3H)- one |
| 62 | 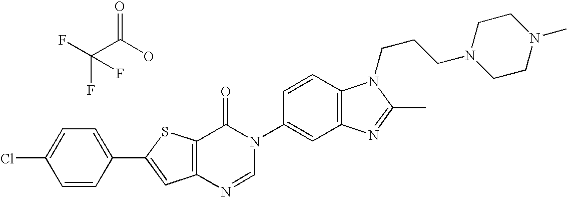 | 6-(4-chlorophenyl)-3-{2-methyl-1- [3-(4-methyl-1- piperazinyl)propyl]-1H- benzimidazol-5-yl}thieno[3,2- d]pyrimidin-4(3H)-one trifluoroacetate |
| 63 | 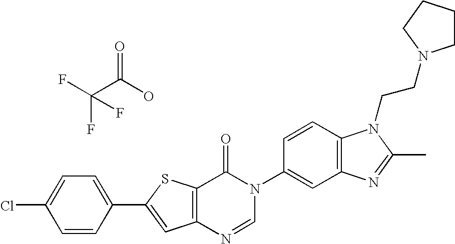 | methyl-1-[2-(1-pyrrolidinyl)ethyl]- 1H-benzimidazol-5-yl}thieno[3,2- d]pyrimidin-4(3H)-one trifluoroacetate |
| 64 | 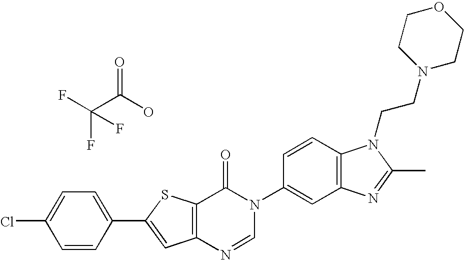 | 6-(4-chlorophenyl)-3-{2-methyl-1- [2-(4-morpholinyl)ethyl]-1H- benzimidazol-5-yl}thieno[3,2- d]pyrimidin-4(3H)-one trifluoroacetate |
| 65 | 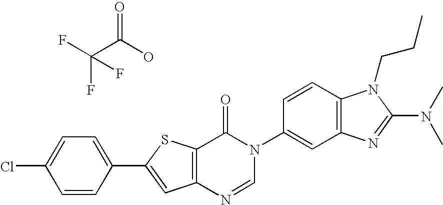 | 6-(4-chlorophenyl)-3-[2- (dimethylamino)-1-propyl-1H- benzimidazol-5-yl]thieno[3,2- d]pyrimidin-4(3H)-one trifluoroacetate |
| 66 | 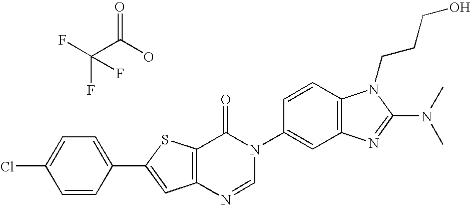 | 6-(4-chlorophenyl)-3-[2- (dimethylamino)-1-(3- hydroxypropyl)-1H-benzimidazol- 5-yl]thieno[3,2-d]pyrimidin-4(3H)- one trifluoroacetate (salt) |
| 67 | 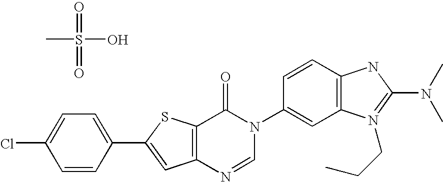 | 6-(4-chlorophenyl)-3-[2- (dimethylamino)-1-propyl-1H- benzimidazol-6-yl]thieno[3,2- d]pyrimidin-4(3H)-one methanesulfonate |
| 68 | 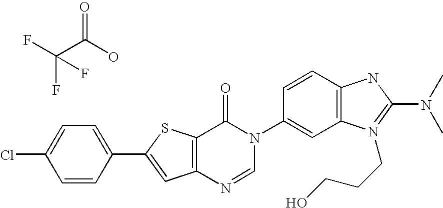 | 6-(4-chlorophenyl)-3-[2- (dimethylamino)-1-(3- hydroxypropyl)-1H-benzimidazol- 6-yl]thieno[3,2-d]pyrimidin-4(3H)- one trifluoroacetate (salt) |
| 69 | 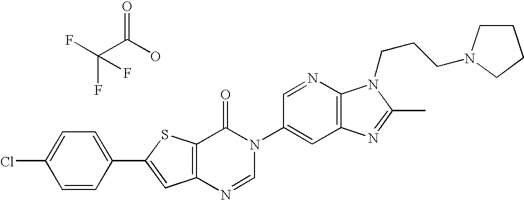 | 6-(4-chlorophenyl)-3-{2-methyl-3- [3-(1-pyrrolidinyl)propyl]-3H- imidazo[4,5-b]pyridin-6- yl}thieno[3,2-d]pyrimidin-4(3H)- one trifluoroacetate |
| 70 | 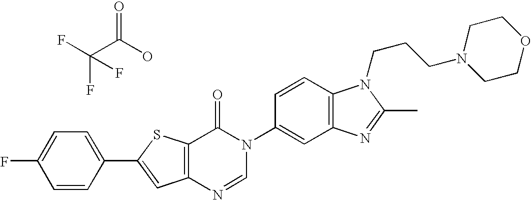 | 6-(4-fluorophenyl)-3-{2-methyl-1- [3-(4-morpholinyl)propyl]-1H- benzimidazol-5-yl}thieno[3,2- d]pyrimidin-4(3H)-one trifluoroacetate |
| 71 | 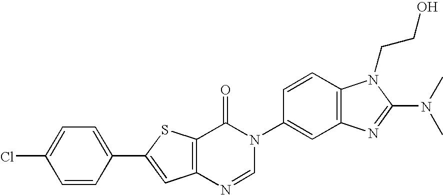 | 6-(4-chlorophenyl)-3-[2- (dimethylamino)-1-(2- hydroxyethyl)-1H-benzimidazol- 5-yl]thieno[3,2-d]pyrimidin-4(3H)- one |
| 72 | 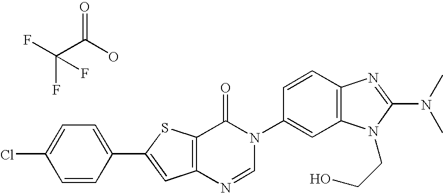 | 6-(4-chlorophenyl)-3-[2- (dimethylamino)-1-(2- hydroxyethyl)-1H-benzimidazol- 6-yl]thieno[3,2-d]pyrimidin-4(3H)- one trifluoroacetate |
| 73 | 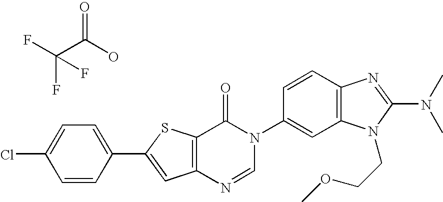 | 6-(4-chlorophenyl)-3-{2- (dimethylamino)-1-[2- (methyloxy)ethyl]-1H- benzimidazol-6-yl}thieno[3,2- d]pyrimidin-4(3H)-one trifluoroacetate |
| 74 | 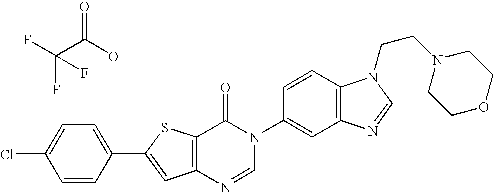 | 6-(4-chlorophenyl)-3-{1-[2-(4- morpholinyl)ethyl]-1H- benzimidazol-5-yl}thieno[3,2- d]pyrimidin-4(3H)-one trifluoroacetate |
| 75 | 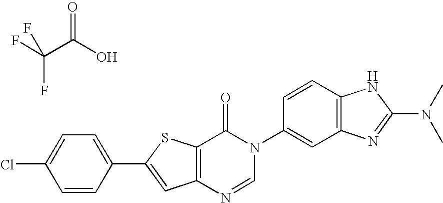 | 6-(4-chlorophenyl)-3-[2- (dimethylamino)-1H- benzimidazol-5-yl]thieno[3,2- d]pyrimidin-4(3H)-one trifluoroacetate |
| 76 | 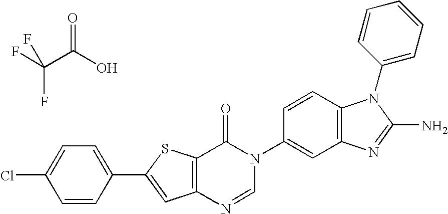 | 3-(2-amino-1-phenyl-1H- benzimidazol-5-yl)-6-(4- chlorophenyl)thieno[3,2- d]pyrimidin-4(3H)-one trifluoroacetate |
| 77 | 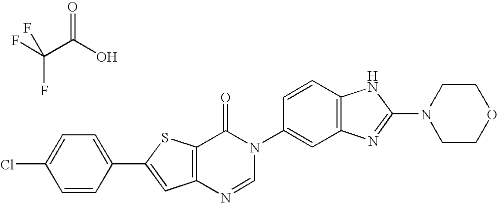 | 6-(4-chlorophenyl)-3-[2-(4- morpholinyl)-1H-benzimidazol-5- yl]thieno[3,2-d]pyrimidin-4(3H)- one trifluoroacetate |
| 78 | 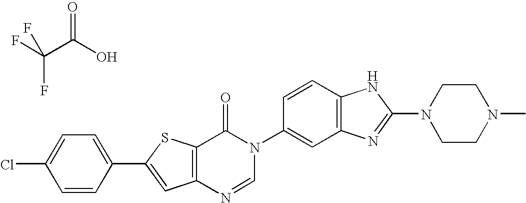 | 6-(4-chlorophenyl)-3-[2-(4- methyl-1-piperazinyl)-1H- benzimidazol-5-yl]thieno[3,2- d]pyrimidin-4(3H)-one trifluoroacetate |
| 79 | 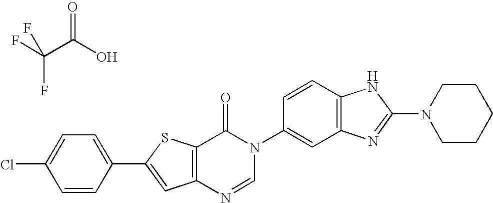 | 6-(4-chlorophenyl)-3-[2-(1- piperidinyl)-1H-benzimidazol-5- yl]thieno[3,2-d]pyrimidin-4(3H)- one trifluoroacetate |
| 80 | 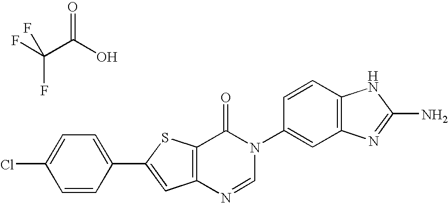 | 3-(2-amino-1H-benzimidazol-5- yl)-6-(4-chlorophenyl)thieno[3,2- d]pyrimidin-4(3H)-one trifluoroacetate |
| 81 | 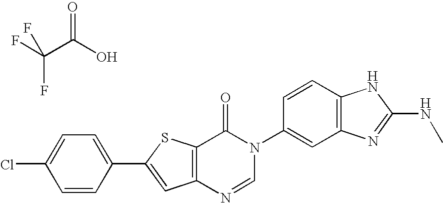 | 6-(4-chlorophenyl)-3-[2- (methylamino)-1H-benzimidazol- 5-yl]thieno[3,2-d]pyrimidin-4(3H)- one trifluoroacetate |
| 82 | 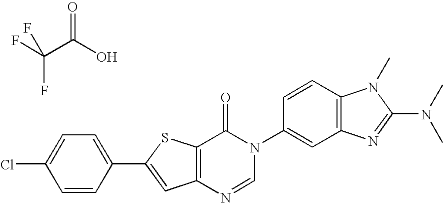 | 6-(4-chlorophenyl)-3-[2- (dimethylamino)-1-methyl-1H- benzimidazol-5-yl]thieno[3,2- d]pyrimidin-4(3H)-one trifluoroacetate |
| 83 | 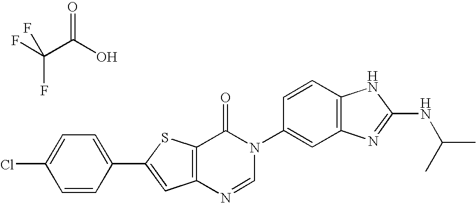 | 6-(4-chlorophenyl)-3-{2-[(1- methylethyl)amino]-1H- benzimidazol-5-yl}thieno[3,2- d]pyrimidin-4(3H)-one trifluoroacetate |
| 84 | 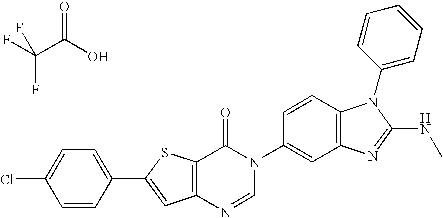 | 6-(4-chlorophenyl)-3-[2- (methylamino)-1-phenyl-1H- benzimidazol-5-yl]thieno[3,2- d]pyrimidin-4(3H)-one trifluoroacetate |
| 85 | 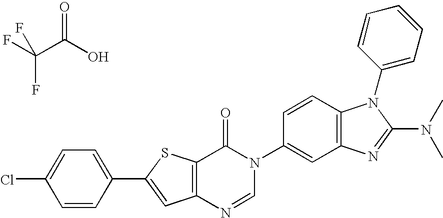 | 6-(4-chlorophenyl)-3-[2- (dimethylamino)-1-phenyl-1H- benzimidazol-5-yl]thieno[3,2- d]pyrimidin-4(3H)-one trifluoroacetate |
| 86 | 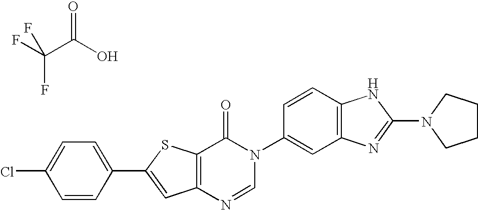 | 6-(4-chlorophenyl)-3-[2-(1- pyrrolidinyl)-1H-benzimidazol-5- yl]thieno[3,2-d]pyrimidin-4(3H)- one trifluoroacetate |
| 87 | 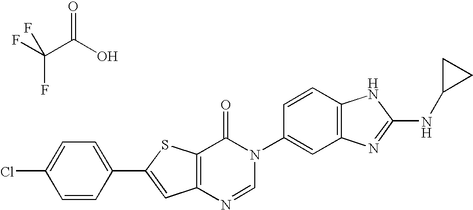 | 6-(4-chlorophenyl)-3-[2- (cyclopropylamino)-1H- benzimidazol-5-yl]thieno[3,2- d]pyrimidin-4(3H)-one trifluoroacetate |
| 88 | 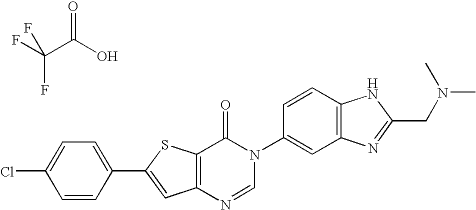 | 6-(4-chlorophenyl)-3-{2- [(dimethylamino)methyl]-1H- benzimidazol-5-yl}thieno[3,2- d]pyrimidin-4(3H)-one trifluoroacetate |
| 89 | 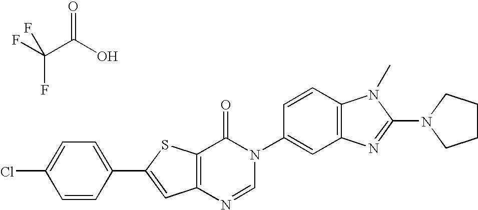 | 6-(4-chlorophenyl)-3-[1-methyl-2- (1-pyrrolidinyl)-1H-benzimidazol- 5-yl]thieno[3,2-d]pyrimidin-4(3H)- one trifluoroacetate |
| 90 | 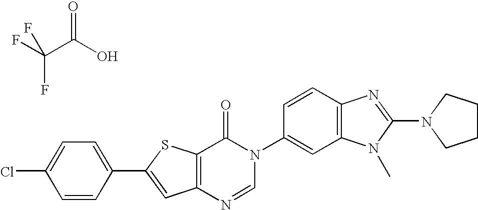 | 6-(4-chlorophenyl)-3-[1-methyl-2- (1-pyrrolidinyl)-1H-benzimidazol- 6-yl]thieno[3,2-d]pyrimidin-4(3H)- one trifluoroacetate |
| 91 | 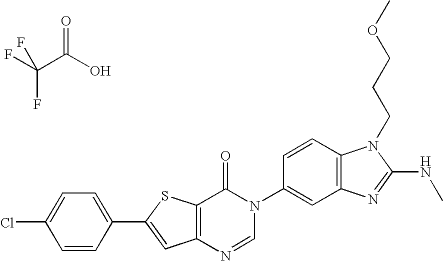 | 6-(4-chlorophenyl)-3-{2- (methylamino)-1-[3- (methyloxy)propyl]-1H- benzimidazol-5-yl}thieno[3,2- d]pyrimidin-4(3H)-one trifluoroacetate |
| 92 | 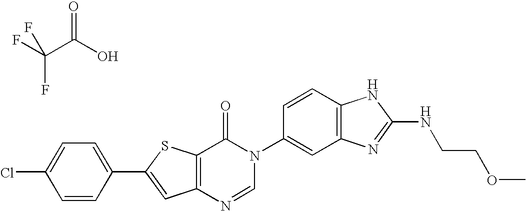 | 6-(4-chlorophenyl)-3-(2-{[2- (methyloxy)ethyl]amino}-1H- benzimidazol-5-yl)thieno[3,2- d]pyrimidin-4(3H)-one trifluoroacetate |
| 93 | 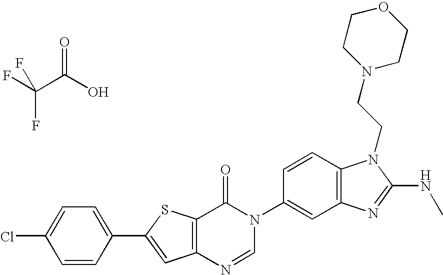 | 6-(4-chlorophenyl)-3-{2- (methylamino)-1-[2-(4- morpholinyl)ethyl]-1H- benzimidazol-5-yl}thieno[3,2- d]pyrimidin-4(3H)-one trifluoroacetate |
| 94 | 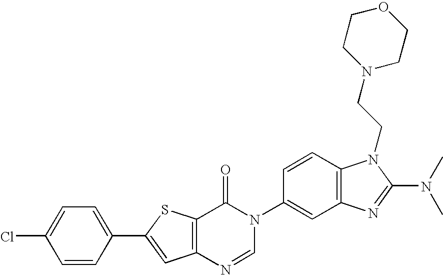 | 6-(4-chlorophenyl)-3-{2- (dimethylamino)-1-[2-(4- morpholinyl)ethyl]-1H- benzimidazol-5-yl}thieno[3,2- d]pyrimidin-4(3H)-one |
| 95 | 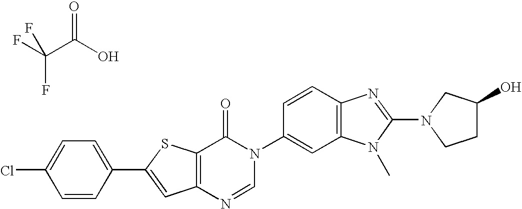 | 6-(4-chlorophenyl)-3-{2-[(3S)-3- hydroxy-1-pyrrolidinyl]-1-methyl- 1H-benzimidazol-6-yl}thieno[3,2- d]pyrimidin-4(3H)-one trifluoroacetate (salt) |
| 96 | 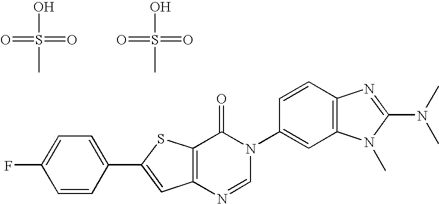 | 3-[2-(dimethylamino)-1-methyl- 1H-benzimidazol-6-yl]-6-(4- fluorophenyl)thieno[3,2- d]pyrimidin-4(3H)-one dimethanesulfonate |
| 97 | 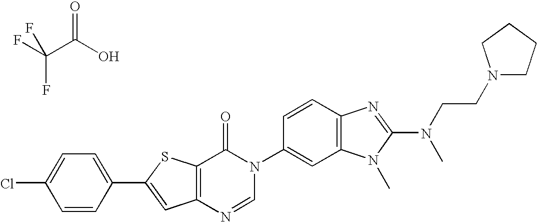 | 6-(4-chlorophenyl)-3-(1-methyl-2- {methyl[2-(1- pyrrolidinyl)ethyl]amino}-1H- benzimidazol-6-yl)thieno[3,2- d]pyrimidin-4(3H)-one trifluoroacetate |
| 98 | 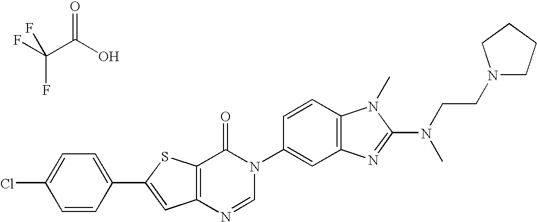 | 6-(4-chlorophenyl)-3-(1-methyl-2- {methyl[2-(1- pyrrolidinyl)ethyl]amino}-1H- benzimidazol-5-yl)thieno[3,2- d]pyrimidin-4(3H)-one trifluoroacetate |
| 99 | 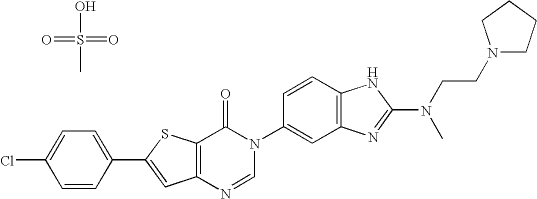 | 6-(4-chlorophenyl)-3-(2- {methyl[2-(1- pyrrolidinyl)ethyl]amino}-1H- benzimidazol-5-yl)thieno[3,2- d]pyrimidin-4(3H)-one methanesulfonate |
| 100 | 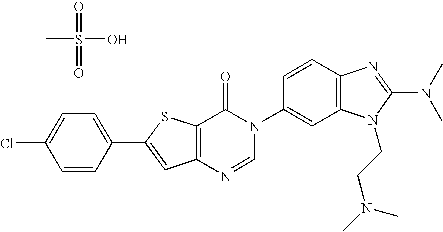 | 6-(4-chlorophenyl)-3-{2- (dimethylamino)-1-[2- (dimethylamino)ethyl]-1H- benzimidazol-6-yl}thieno[3,2- d]pyrimidin-4(3H)-one methanesulfonate |
| 101 | 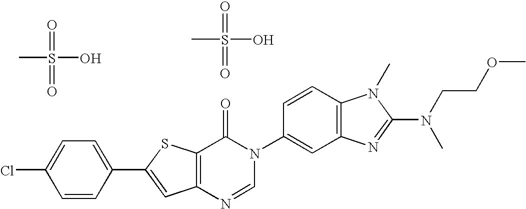 | 6-(4-chlorophenyl)-3-(1-methyl-2- {methyl[2- (methyloxy)ethyl]amino}-1H- benzimidazol-5-yl)thieno[3,2- d]pyrimidin-4(3H)-one dimethanesulfonate |
| 102 | 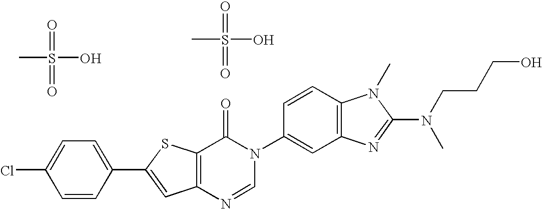 | 6-(4-chlorophenyl)-3-{2-[(3- hydroxypropyl)(methyl)amino]-1- methyl-1H-benzimidazol-5- yl}thieno[3,2-d]pyrimidin-4(3H)- one dimethanesulfonate |
| 103 | 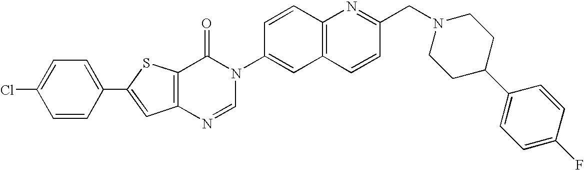 | 6-(4-chlorophenyl)-3-(2-{[4-(4- fluorophenyl)piperidin-1- yl]methyl}quinolin-6-yl)thieno[3,2- d]pyrimidin-4(3H)-one |
| 104 | 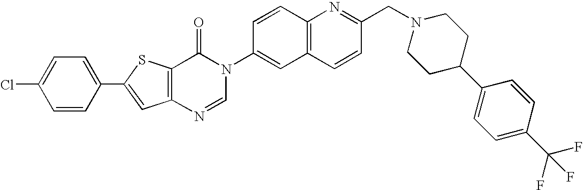 | 6-(4-chlorophenyl)-3-[2-({4-[4- (trifluoromethyl)phenyl]piperidin- 1-yl}methyl)quinolin-6- yl]thieno[3,2-d]pyrimidin-4(3H)- one |
| 105 | 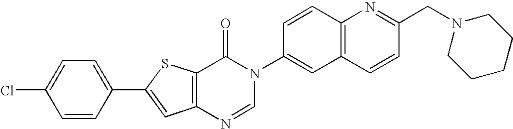 | 6-(4-chlorophenyl)-3-[2- (piperidin-1-ylmethyl)quinolin-6- yl]thieno[3,2-d]pyrimidin-4(3H)- one |
| 106 | 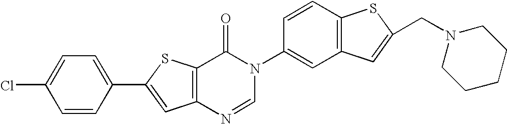 | 6-(4-chlorophenyl)-3-[2- (piperidin-1-ylmethyl)-1- benzothien-5-yl]thieno[3,2- d]pyrimidin-4(3H)-one |
| 107 | 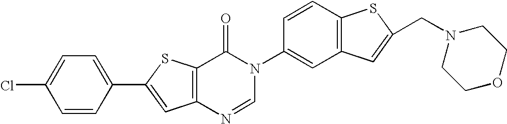 | 6-(4-chlorophenyl)-3-[2- (morpholin-4-ylmethyl)-1- benzothien-5-yl]thieno[3,2- d]pyrimidin-4(3H)-one |
| 108 | 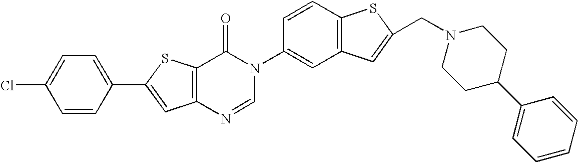 | 6-(4-chlorophenyl)-3-{2-[(4- phenylpiperidin-1-yl)methyl]-1- benzothien-5-yl}thieno[3,2- d]pyrimidin-4(3H)-one |
| 109 | 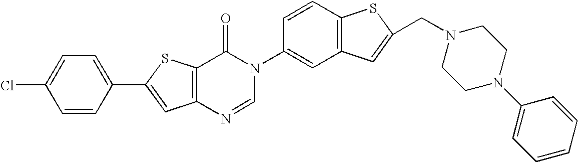 | 6-(4-chlorophenyl)-3-{2-[(4- phenylpiperazin-1-yl)methyl]-1- benzothien-5-yl}thieno[3,2- d]pyrimidin-4(3H)-one |
| 110 | 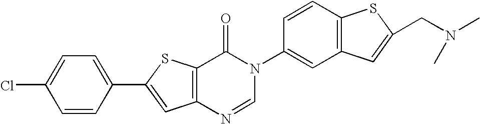 | 6-(4-chlorophenyl)-3-{2- [(dimethylamino)methyl]-1- benzothien-5-yl}thieno[3,2- d]pyrimidin-4(3H)-one |
| 111 | 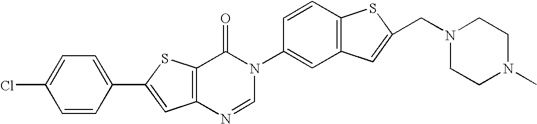 | 6-(4-chlorophenyl)-3-{2-[(4- methylpiperazin-1-yl)methyl]-1- benzothien-5-yl}thieno[3,2- d]pyrimidin-4(3H)-one |
| 112 | 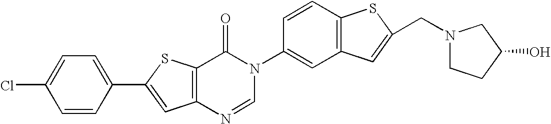 | 6-(4-chlorophenyl)-3-(2-{[(3R)-3- hydroxypyrrolidin-1-yl]methyl}-1- benzothien-5-yl)thieno[3,2- d]pyrimidin-4(3H)-one |
| 113 | 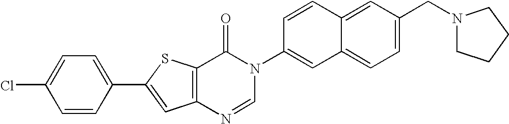 | 6-(4-chlorophenyl)-3-[6- (pyrrolidin-1-ylmethyl)-2- naphthyl]thieno[3,2-d]pyrimidin- 4(3H)-one |
| 114 | 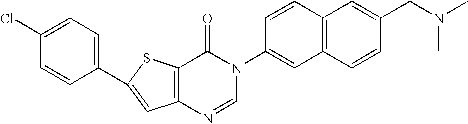 | 6-(4-chlorophenyl)-3-{6- [(dimethylamino)methyl]-2- naphthalenyl}thieno[3,2- d]pyrimidin-4(3H)-one |
| 115 | 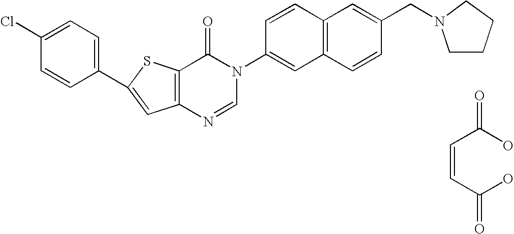 | 6-(4-chlorophenyl)-3-[6-(1- pyrrolidinylmethyl)-2- naphthalenyl]thieno[3,2- d]pyrimidin-4(3H)-one maleate salt |
| 116 | 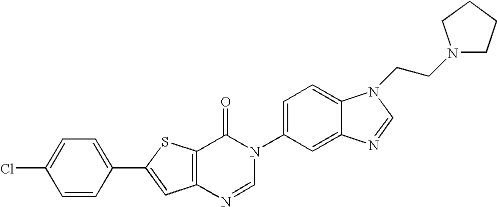 | 6-(4-chlorophenyl)-3-{1-[2-(1- pyrrolidinyl)ethyl]-1H- benzimidazol-5-yl}thieno[3,2- d]pyrimidin-4(3H)-one |
| 117 | 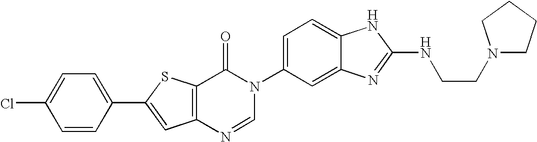 | 6-(4-chlorophenyl)-3-(2-{[2-(1- pyrrolidinyl)ethyl]amino}-1H- benzimidazol-5-yl)thieno[3,2- d]pyrimidin-4(3H)-one |
| 118 | 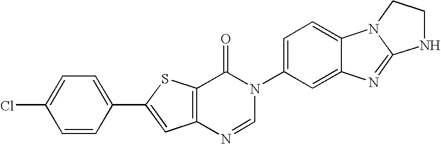 | 6-(4-chlorophenyl)-3-(2,3- dihydro-1H-imidazo[1,2- a]benzimidazol-7-yl)thieno[3,2- d]pyrimidin-4(3H)-one |

It will be appreciated by those skilled in the art that the compounds of the present invention may also be utilized in the form of a pharmaceutically acceptable salt or solvate or physiologically functional derivative thereof (e.g., a prodrug). The pharmaceutically acceptable salts of the compounds of formula (I) include conventional salts formed from pharmaceutically acceptable inorganic or organic acids or bases as well as quaternary ammonium salts. More specific examples of suitable acid salts include maleic, hydrochloric, hydrobromic, sulfuric, phosphoric, nitric, perchloric, fumaric, acetic, propionic, succinic, glycolic, formic, lactic, aleic, tartaric, citric, palmoic, malonic, hydroxymaleic, phenylacetic, glutamic, benzoic, salicylic, fumaric, toluenesulfonic, methanesulfonic (mesylate), naphthaliene-2-sulfonic, benzenesulfonic, hydroxynaphthoic, hydroiodic, malic, steroic, tannic, and the like.

Other acids such as oxalic, while not in themselves pharmaceutically acceptable, may be useful in the preparation of salts useful as intermediates in obtaining the compounds of the invention and their pharmaceutically acceptable salts. More specific examples of suitable basic salts include sodium, lithium, potassium, magnesium, aluminum, calcium, zinc, N,N′-dibenzylethylenediamine, chloroprocaine, choline, diethanolamine, ethylenediamine, N-methylglucamine and procaine salts.

The term “solvate” as used herein refers to a complex of variable stoichiometry formed by a solute (a compound of formula (I)) and a solvent. Solvents, by way of example, include water, methanol, ethanol, and acetic acid.

The term “physiologically functional derivative” as used herein refers to any pharmaceutically acceptable derivative of a compound of the present invention, for example, a ester or an amide of a compound of formula (I), which upon administration to an animal, particularly a mammal, such as a human, is capable of providing (directly or indirectly) a compound of the present invention or an active metabolite thereof. See, for example, Burger's Medicinal Chemistry and Drug Discovery, 5^{th} Edition, Vol. 1: Principles and Practice.

Processes for preparing pharmaceutically salts, solvates, and physiologically functional derivatives of the compounds of formula (I) are conventional in the art. See, e.g., Burger's Medicinal Chemistry and Drug Discovery,5^{th} Edition, Vol. 1: Principles and Practice.

Compounds of formula (I) below are conveniently prepared in accordance with the reaction schemes and/or processes outlined or described herein.

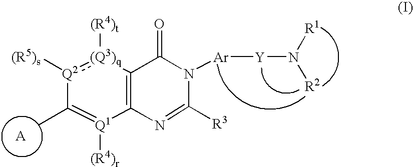

As will be apparent to those skilled in the art, in the processes described below for the preparation of compounds of formula (I), certain intermediates, may be in the form of pharmaceutically salts, solvates or physiologically functional derivatives of the compound. With respect to any intermediate employed in the process of preparing compounds of formula (I), the terms or identifiers have the same meanings as noted above with respect to compounds of formula (I). In general, processes for preparing pharmaceutically acceptable salts, solvates and physiologically functional derivatives of intermediates are known, and the process for preparing pharmaceutically acceptable salts, solvates and physiological functional derivatives of the compounds of formula (I) are similar and set forth below.

Unless otherwise stated, \textcircled{A}, R⁵, R⁴, R³, R², R¹, Ar, Y, Q¹, Q², Q³, q, r, s, and t are as defined in formula (I) for all of the processes enumerated herein.

Thus, compounds of formula (I) wherein R⁵ is H may be prepared by reaction of an aniline of formula (II) with a formamidine ester of formula (III) wherein R is C₁₋₄ alkyl.

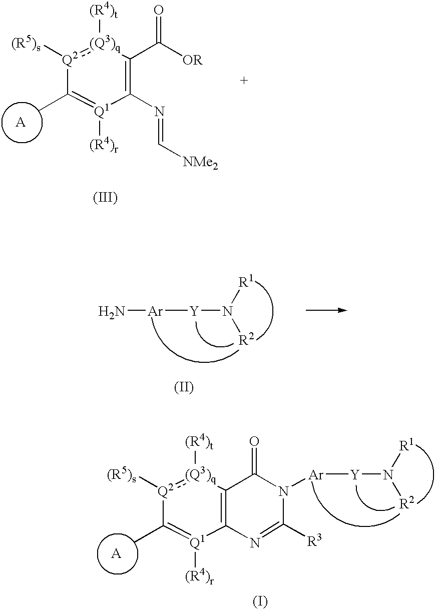

Compounds of formula (I) can also be prepared by an amide coupling of the corresponding amino acid (IV) and the desired aniline (II) in a solvent, such as methylene chloride, with amide coupling agents such as EDCI (1-[3-(dimethylamino)propyl]-3-ethylcarbodiimide hydrochloride), followed by cyclization in refluxing carboxylic acids, such as formic acid.

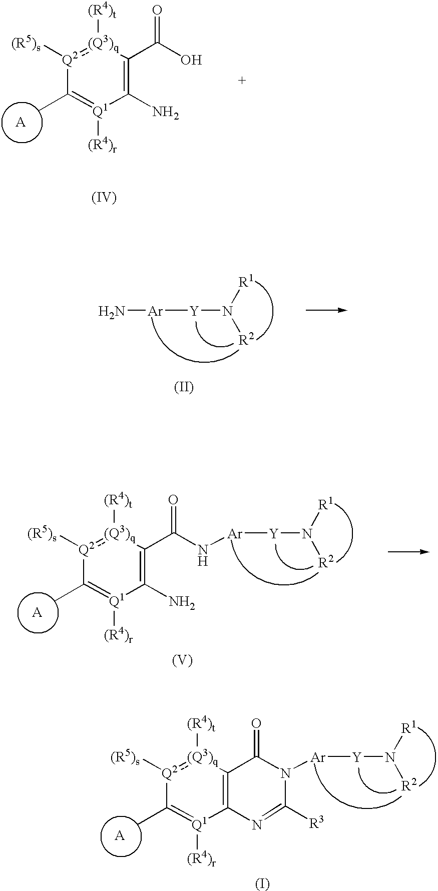

Compounds of formula (I) may also be prepared by reaction of a compound of formula (Va) 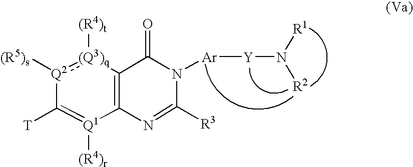
with a compound capable of introducing the group \textcircled{A}, and T is a leaving group (e.g., chloro, bromo, iodo, and triflate (—OSO₂CF₃)).

Thus compounds of formula (I) may be prepared from the compound of formula (Va) with a boronic acid and a palladium catalyst using a Suzuki coupling reaction or with an organostannane reagent and a palladium catalyst using a Stille coupling reaction.

Compounds of formula (I) may also be prepared by reaction of an amino ester of formula (III) wherein R is C₁₋₄ alkyl with an aniline of formula (II) in a solvent such as dichloromethane or 1,2-dichloroethane in the presence of trimethylaluminum to produce a compound of formula (Vb) and cyclizing said compound of formula (Vb).

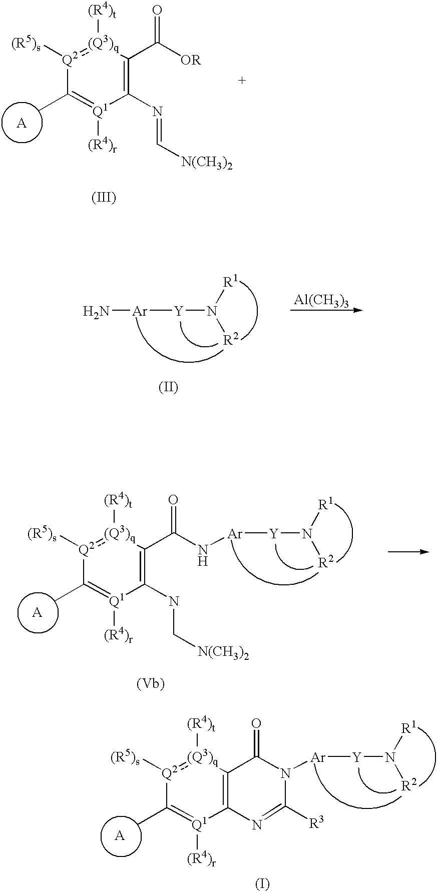

Compounds of formula (I) wherein R³ is hydrogen may also be prepared by reaction of a sulfur-containing compound such as (VI) with a reductant, such as Raney Nickel, in a solvent such as ethanol.

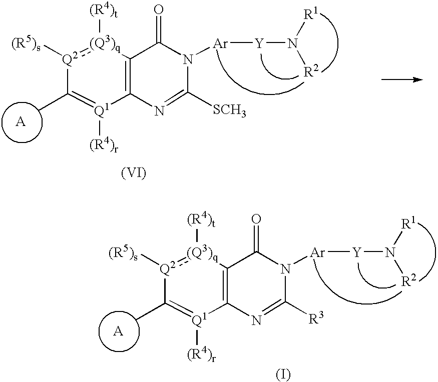

Compounds of formula (II) may be prepared by reduction of the corresponding nitroaromatic (VII) using hydrogen and a catalyst (e.g., 10% Pd on carbon), stannous chloride, or sodium dithionite.

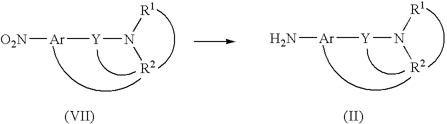

Compounds of formula VIIc wherein Y is CH₂ can be prepared from a compound (VIIa) and an amine (VIIb) and T is a leaving group (e.g., Cl, Br, I, mesylate, and tosylate).

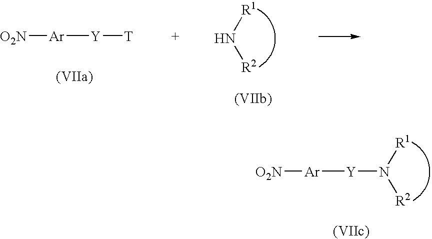

Alternatively, compounds of this type can be made by reductive amination of an aldehyde of formula (VIII) by an amine of formula (VIIb) in the presence of a reducing agent such as a sodium borohydride.

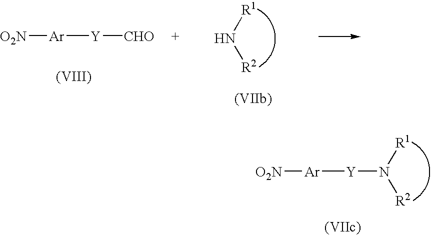

Compounds of formula (VIII) in which Y is a bond can be prepared by reaction of a compound of formula (IX) (in which Y is a bond) with an oxidant such as selenium dioxide in a solvent such as dioxane.

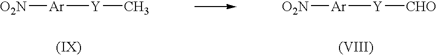

Compounds of formula VIIc can be prepared from a compound (VIIa) and an amine (VIIb) in which T is a leaving group.

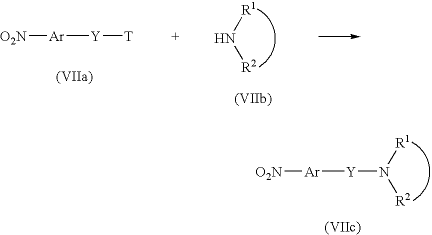

Compounds of formula (II) can be prepared from a compound (IX) and an amine (VIIb) in which T is a leaving group.

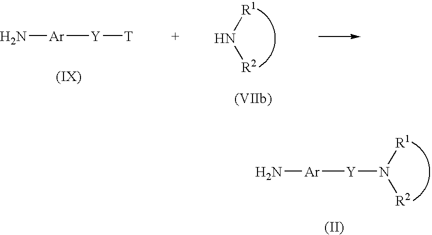

In the present invention, the compounds of formula (I) are believed to have a role in the treatment of depression, anxiety, obesity and/or diabetes. Compounds of the present invention are antagonists of a MCHR1 and can be used for the treatment of a disease caused by or attributable to a melanin-concentrating hormone. Compounds of the invention may reduce hunger, suppress appetite, control eating, and/or induce satiety.

The present invention provides methods for the treatment of several conditions or diseases such as obesity, diabetes, depression (eg., major depression and/or bipolar disorder), and/or anxiety. Such treatment comprises the step of administering a therapeutically effective amount of the compound of formula (I), including a salt, solvate, or physiologically functional derivative thereof to a mammal, preferably a human. Such treatment can also comprise the step of administering a therapeutically effective amount of a pharmaceutical composition containing a compound of formula (I), including a salt, solvate, or physiologically functional derivative thereof to a mammal, preferably a human. As used herein, the term “treatment” refers to alleviating the specified condition, eliminating or reducing one or more symptoms of the condition, slowing or eliminating the progression of the condition, and preventing or delaying the reoccurrence of the condition in a previously afflicted or diagnosed patient or subject.

As used herein, the term “therapeutically effective amount” means an amount of a compound of formula (I) which is sufficient, in the subject to which it is administered, to elicit the biological or medical response of a cell culture, tissue, system, animal (including human) that is being sought, for instance, by a researcher or clinician.

The precise therapeutically effective amount of the compounds of formula (I) will depend on a number of factors including, but not limited to, the age and weight of the subject being treated, the precise disorder requiring treatment and its severity, the nature of the formulation, and the route of administration, and will ultimately be at the discretion of the attendant physician or veterinarian. Typically, the compound of formula (I) will be given for treatment in the range of about 0.1 to about 200 mg/kg body weight of recipient (animal) per day and more usually in the range of about 1 to about 100 mg/kg body weight per day. In general, acceptable daily dosages, may be from about 0.1 to about 5000 mg/day, and preferably from about 0.1 to about 2000 mg/day. Unit doses will normally be administered once or more than once per day, preferably about 1 to about 4 times per day.

The administration of compounds of the invention to an animal, particularly a mammal such as a human, may be by way of oral (including sub-lingual), parenteral, nasal, rectal or transdermal administration. Preferably oral administration is employed.

While it is possible that, for use in therapy, a therapeutically effective amount of a compound of formula (I) may be administered as the raw chemical, it is typically presented as the active ingredient of a pharmaceutical composition or formulation. Accordingly, the invention further provides a pharmaceutical composition comprising a compound of formula (I). The pharmaceutical composition may further comprise one or more pharmaceutically acceptable carriers, diluents, and/or excipients. The carrier(s), diluent(s), and/or excipient(s) must be acceptable in the sense of being compatible with the other ingredients of the formulation and not deleterious to the recipient thereof.

In accordance with another aspect of the invention there is also provided a process for the preparation of a pharmaceutical formulation including admixing a compound of formula (I) with one or more pharmaceutically acceptable carriers, diluents, and/or excipients.

Pharmaceutical formulations may be presented in unit dose form containing a predetermined amount of active ingredient per unit dose. Such a unit may contain a therapeutically effective dose of the compound of formula (I) or a fraction of a therapeutically effective dose such that multiple unit dosage forms might be administered at a given time to achieve the desired therapeutically effective dose. Preferred unit dosage formulations are those containing a daily dose or sub-dose, as herein above recited, or an appropriate fraction thereof, of an active ingredient. Furthermore, such pharmaceutical formulations may be prepared by any of the methods well known in the pharmacy art.

Pharmaceutical formulations may be adapted for administration by any appropriate route, for example, by the oral (including buccal or sublingual), rectal, nasal, topical (including buccal, sublingual, or transdermal), vaginal or parenteral (including subcutaneous, intramuscular, intravenous or intradermal) route. Such formulations may be prepared by any method known in the art of pharmacy, for example, by bringing into association the active ingredient with the carrier(s), diluent(s), and/or excipient(s).

Pharmaceutical formulations adapted for oral administration may be presented as discrete units such as capsules (including soft gelatin capsules, hard gelatin capsules, and capsules made from other polymers such as hydroxypropylmethylcellulose) or tablets; powders or granules; solutions, emulsions, or suspensions in aqueous or non-aqueous liquids; edible foams or whips; or oil-in-water liquid emulsions or water-in-oil emulsions. For instance, for oral administration in the form of a tablet or capsule (e.g., hard, soft, elastic, gelatinous and/or non-gelatinous), the active drug component can be combined with an oral, non-toxic pharmaceutically acceptable inert carrier such as ethanol, glycerol, water, and the like. Powders are prepared by comminuting the compound to a suitable fine size and mixing with a similarly comminuted pharmaceutical carrier such as an edible carbohydrate, as, for example, starch or mannitol. Flavoring, preservative, opaque, dispersing and coloring agent or dye can also be present.

Capsules are made by preparing a powder mixture as described above, and filling formed gelatin and/or non-gelatinous sheaths. Glidants and lubricants, such as colloidal silica, talc, magnesium stearate, calcium stearate or solid polyethylene glycol can be added to the powder mixture before the filling operation. A disintegrating or solubilizing agent such as agar-agar, calcium carbonate or sodium carbonate can also be added to improve the availability of the medicament when the capsule is ingested.

Moreover, when desired or necessary, suitable binders, lubricants, disintegrating agents and coloring agents can also be incorporated into the mixture. Suitable binders include starch, gelatin, natural sugars such as glucose or beta-lactose, corn sweeteners, natural and synthetic gums such as acacia, tragacanth or sodium alginate, cellulosic polymers (e.g., hydrogels (HPMC, HPC, PVA), and the like), carboxymethylcellulose, polyethylene glycol, waxes, polyvinylpyrrolidone, and the like. Lubricants used in these dosage forms include sodium oleate, sodium stearate, magnesium stearate, sodium benzoate, sodium acetate, sodium chloride and the like. Disintegrators (disintegrants) include, without limitation, starch, methyl cellulose, agar, bentonite, xanthan gum, and the like. Tablets are formulated, for example, by preparing a powder mixture, granulating or slugging, adding a lubricant and disintegrant and pressing into tablets. A powder mixture is prepared by mixing the compound, suitably comminuted, with a diluent or base as described above, and optionally, with a binder such as carboxymethylcellulose, an aliginate, gelatin, or polyvinyl pyrrolidone, a solution retardant such as paraffin, a resorption accelerator such as a quaternary salt and/or an absorption agent such as bentonite, kaolin or dicalcium phosphate. The powder mixture can be granuated by wetting with a binder such as a syrup, starch paste, acadia mucilage or solutions of cellulosic or polymeric materials and forcing through a screen. As an alternative to granulating, the powder mixture can be run through the tablet machine and the result is imperfectly formed slugs broken into granules. The granules can be lubricated to prevent sticking to the tablet forming dies by means of the addition of stearic acid, a stearate salt, talc or mineral oil. The lubricated mixture is then compressed into tablets. The compounds of the present invention can also be combined with a free flowing inert carrier and compressed into tablets directly without going through the granulating or slugging steps. A clear or opaque protective coating consisting of a sealing coat of shellac, a coating of sugar or polymeric material (e.g., HPMC) and a polish coating of wax can be provided. Dyestuffs can be added to these coatings to distinguish different unit dosages.

The drug may be dissolved or dispersed in a volatile liquid such as water or ethanol and sprayed onto nonpareil beads. A binder such as sucrose, polyvinylpyrollidone, hydroxypropylmethylcellulose, or the like may be used. After at least one coating, protective coat(s) of a polymer such as hydroxypropylmethylcellulose may be applied and/or a sustained or delayed release coating(s) may be applied. Such coated beads may optionally be compressed into tablets or filled into capsules.

Oral fluids such as solution, syrups and elixirs can be prepared in dosage unit form so that a given quantity contains a predetermined amount of active ingredient. Syrups can be prepared by dissolving the compound in a suitably flavored aqueous solution, while elixirs are prepared through the use of a non-toxic alcoholic vehicle. Suspensions can be formulated by dispersing the compound in a non-toxic vehicle. Solubilizers and emulsifiers such as ethoxylated isostearyl alcohols and polyoxy ethylene sorbitol ethers, preservatives, flavor additive such as peppermint oil or natural sweeteners or saccharin or other artificial sweeteners, and the like can also be added.

Where appropriate, dosage unit formulations for oral administration can be microencapsulated. The formulation can also be prepared to prolong or sustain the release as for example by coating or embedding particulate material in polymers, wax, or the like. The compound of formula (I) can also be incorporated into a candy, a wafer, and/or tongue tape formulation for administration as a “quick-dissolve” medicament. Oral dosage forms may be taken with or without water.

Additionally, the present invention comprises a compound of formula (I) in combination with at least one other species selected from the group consisting of at least one agent or drug for treating obesity, diabetes (e.g., rosiglitazone and/or metformin), hypertension, and arteriosclerosis. In particular, a compound of formula (I) may be combined with at least one species for the treatment of obesity selected from the group of human ciliary neurotrophic factor, a CB-1 antagonist or inverse agonist (such as rimonabant), a neurotransmitter reuptake inhibitor (such as sibutramine, bupropion, or bupropion HCl), a lipase inhibitor (such as orlistat), an MC4R agonist, a 5-HT2c agonist, and a ghrelin receptor agonist or antagonist.

Also, the invention can be the use of a compound of formula (I) for the manufacture of a medicine (that is, medicament) for the treatment of a condition selected from the group consisting of obesity, diabetes, depression, and anxiety in a mammal.

The following examples are intended for illustration only and are not intended to limit the scope of the invention in any way, the invention being defined by the claims which follow. All references cited in this specification are hereby incorporated by reference.

Reagents are commercially available or are prepared according to procedures in the literature.

Experimental Section

### EXAMPLE 1

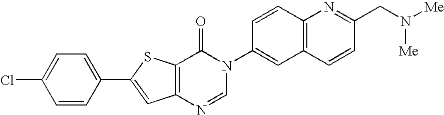

### 6-(4-chlorophenyl)-3-{2-[(dimethylamino)methyl]quinolin-6-yl}thieno[3,2-d]pyrimidin-4(3H)-one

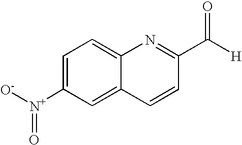

### Step A: 6-nitroquinoline-2-carbaldehyde

To a hot solution of selenium dioxide (41.6 g, 375 mmol) in dioxane (185 mL) and water (35 mL) was added 2-methyl-6-nitroquinoline (47.0 g, 250 mmol). The mixture was refluxed for 30 minutes. The selenium black was filtered off and the filtrate was concentrated by rotary evaporation. The resulting solid was filtered, washed with a saturated solution of sodium bicarbonate and then water, and dried to give the product as a tan solid (44.8 g, 89%). ¹H NMR (300 MHz, DMSO-d₆) δ 10.17 (s, 1H), 9.21 (d, J=2.6 Hz, 1H), 8.97 (d, J=8.5 Hz, 1H), 8.59 (dd, J=2.6 Hz, J′=9.2 Hz, 1H), 8.44 (d, J=9.2 Hz, 1H), 8.16 (d, J=8.5 Hz, 1H).

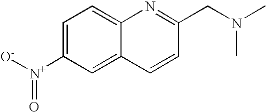

### Step B: N,N-dimethyl-1-(6-nitroquinolin-2-yl)methanamine

To a solution of 6-nitroquinoline-2-carbaldehyde (the intermediate produced in Example 1, Step A; 44.8 g, 221 mmol) in dichloroethane (800 mL) and methanol (320 mL) was added dimethylamine (221 mL, 442 mmol, 2 M in THF) and acetic acid (13.3 g, 221 mmol). The mixture was stirred at room temperarture for 20 min at which point sodium triacetoxyborohydride (65.6 g, 309 mmol) was added in 3 portions with vigorous mechanical stirring. The reaction mixture was stirred overnight. To the reaction mixture was added saturated sodium bicarbonate solution (300 mL) and the mixture was extracted with dichloromethane (2×400 mL). The organic layer was filtered through Celite. The filtrate was washed with brine (200 mL), dried and concentrated to give a tan solid (41.1 g, 80% yield). ¹H NMR (300 MHz, DMSO-d₆) δ 9.05 (d, J=2 Hz), 8.67 (d, J=9.6 Hz, 1H), 8.43 (dd, J=2.2 Hz, J′=12.3 Hz), 8.17 (d, J=9.2 Hz, 1H), 7.82 (d, J=8.6 Hz, 1H), 3.75 (s, 2H), 2.24 (2, 6H).

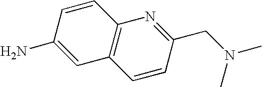

### Step C: 2-[(dimethylamino)methyl]-6-quinolinamine

N,N-Dimethyl(6-nitro-2-quinolinyl)methanamine (the intermediate produced in Example 1, Step B; 365 mg, 1.58 mmol) was dissolved in EtOH. A catalytic amount of Pd/C was added. The mixture was degassed and was stirred under 1 atm H₂ for 5 h. The mix was filtered through celite and the solvents were removed to give the desired intermediate (290 mg, 91%). ¹H NMR (CDCl₃): δ 2.30 (6H, s), 3.68 (2H, s), 6.87 (1H, m), 7.11 (1, m), 7.15 (1H, s), 7.43 (1H, d, J=8.8 Hz), 7.86 (1H, m). LCMS m/z=202 (m +H⁺).

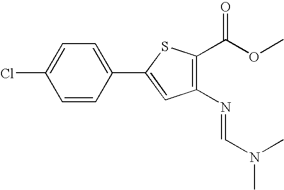

### Step D: methyl 5-(4-chlorophenyl)-3-{[(E)-(dimethylamino)methylidene]amino}-2-thiophenecarboxylate

A mixture of methyl 3-amino-5-(4-chlorophenyl)-2-thiophenecarboxylate (37.3 mmol, 10.0 g) and N,N-dimethylformamide dimethyl acetal (74.7 mmol, 8.9 g) in ethanol (350 mL) was heated to reflux for 3 hours. The solvent was removed by rotary evaporation. To the residue 15 mL of toluene was added and the solvent was removed by rotary evaporation. This was repeated three times. To the resulting sticky residue, 20 mL hexanes were added followed by the gradual addition of ethyl acetate at 0° C. until it solidified. The resulting solid was collected by filtration giving the desired intermediate (11.9 g, 98.9%). ¹H NMR (CDCl₃): δ 3.08 (6H, d, J=6.5 Hz), 3.81 (3H, s), 6.98 (1H, s), 7.35 (2H, d, J=8.6 Hz), 7.53 (2H, d, J=8.5 Hz), 7.69 (1H, s). LCMS m/z=323 (m+H⁺).

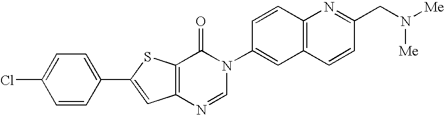

### Step E: 6-(4-chlorophenyl)-3-{2-[(dimethylamino)methyl]quinolin-6-yl}thieno[3,2-d]pyrimidin-4(3H)-one

A 2 M solution of AlMe₃ in hexanes (0.96 mL, 1.92 mmol) was added slowly to a solution of 2-[(dimethylamino)methyl]quinolin-6-amine (the intermediate produced in Example 1, Step C; 0.34 g, 1.69 mmol) in dichloroethane (6 mL) at room temperature under N₂. After 15 min, a solution of methyl 5-(4-chlorophenyl)-3-{[(1E)-(dimethylamino)methylidene]amino}thiophene-2-carboxylate (the intermediate produced in Example 1, Step D; 0.50 g, 1.54 mmol) in dichloroethane (3 mL) was added and stirred at room temperature for 0.5 h. The solution was heated to reflux for 3 h then cooled to room temperature. Formic acid (6 mL) was added carefully and the mixture was heated to reflux for 4 h. Upon cooling to room temperature, an aqueous 1 N NaOH solution (50 mL) was added followed by CH₂Cl₂ (400 mL) and water (300 mL). The organic layer was separated, dried over MgSO₄, filtered and concentrated to give 6-(4-chlorophenyl)-3-{2-[(dimethylamino)methyl]quinolin-6-yl}thieno[3,2-d]pyrimidin-4(3H)-one (the title compound) as a tan solid (0.79 g) with ca. 85% purity. The solid was partially dissolved in hot CHCl₃ (20 mL), filtered, and concentrated. The resulting solid was dissolved in CHCl₃ (15 mL) and then Et₂O (25 mL) was added which produced a white precipitate. The solid was filtered and dried under vacuum to give 6-(4-chlorophenyl)-3-{2-[(dimethylamino)methyl]quinolin-6-yl}thieno[3,2-d]pyrimidin-4(3H)-one (the title compound) as a white powder (0.33 g, 48%). The remaining impure material was subsequently purified in the same manner as described above to yield an additional 0.10 g of the title compound (63% overall yield). ¹H NMR (400 MHz, CDCl₃) δ 8.26 (d, J=7.9 Hz, 1H), 8.25 (s, 1H), 8.21 (d, J=8.2 Hz, 1H), 7.91 (d, J=2.3 Hz, 1H), 7.76 (dd, J=2.4, 9.0 Hz, 1H), 7.72 (d, J=8.4 Hz, 1H), 7.68 (d, J=8.8 Hz, 2H), 7.57 (s, 1H), 7.46 (d, J=8.8 Hz, 2H), 3.83 (s, 2H), 2.37 (s, 6H). EI-LCMS m/z 447 (M+H).

### EXAMPLE 2

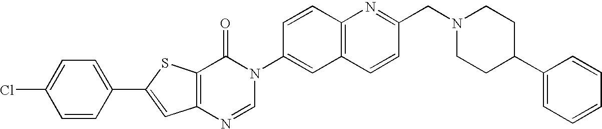

### 6-(4-chlorophenyl)-3-{2-[(4-phenylpiperidin-1-yl)methyl]quinolin-6-yl}thieno[3,2-d]pyrimidin-4(3H)-one

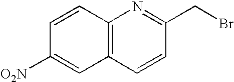

### Step A: 2-(bromomethyl)-6-nitroquinoline

A solution of 2-methyl-6-nitroquinoline (3.0 g, 15.9 mmol) and N-bromosuccinimide (3.11 g, 17.49 mmol) in 36 mL chloroform in a pyrex round bottomed flask was stirred in the presence of a UV lamp at 40° C. for 2 d . After cooling, the mixture was washed with aqueous sodium bicarbonate solution. The aqueous layer was extracted with dichloromethane and the combined organic layers dried over sodium sulfate. Concentration followed by column chromatography on silica gel using hexane:ethyl acetate 7:3 afforded 2-(bromomethyl)-6-nitroquinoline as pale yellow solid (2.67 g, 63%). ¹H NMR (300 MHz, DMSO-d₆) δ 9.10 (s, 1H), 8.78 (d, J=8.6 Hz, 1H), 8.52 (d, J=9.8 Hz, 1H), 8.23 (d, J=9.2 Hz, 1H), 7.92 (d, J=8.5 Hz, 1H), 4.93 (s, 2H); ES-LCMS m/z 267 (M+H).

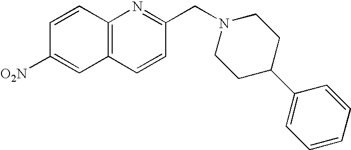

### Step B: 6-nitro-2-[(4-phenylpiperidin-1-yl)methyl]quinoline

To a solution of 2-(bromomethyl)-6-nitroquinoline (the intermediate produced in Example 2, Step A; 1.0 g, 3.76 mmol) in THF at room tempreature was added Hunig's base (1.31 mL, 7.52 mmol) followed by the addition of 4-phenylpiperidine (0.61 g, 3.76 mmol). The contents were stirred for 3 h at room temperature. The crude reaction mixture was concentrated and loaded directly over a silica gel column using hexane:ethyl acetate 1:1 as the eluent to afford 6-nitro-2-[(4-phenylpiperidin-1-yl)methyl]quinoline as a brown solid (1.05 g, 81%). ¹H NMR (300 MHz, DMSO-d₆) δ 9.03 (s, 1H), 8.67 (d, J=9.0 Hz, 1H), 8.43 (d, J=9.4 Hz, 1H), 8.16 (d, J=9.4 Hz, 1H), 7.87 (d, J=8.7 Hz, 1H), 7.29-7.14 (m, 5H), 3.83 (s, 2H), 2.94 (m, 2H), 2.51 (m, 2H), 2.23 (m, 2H), 1.73-1.67 (m, 3H); ES-LCMS m/z 348 (M+H).

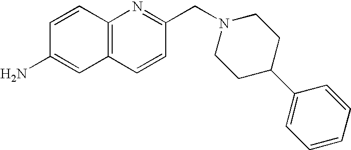

### Step C: 2-[(4-phenylpiperidin-1-yl)methyl]quinolin-6-amine

To a solution of 6-nitro-2-[(4-phenylpiperidin-1-yl)methyl]quinoline (the intermediate produced in Example 2, Step B; 1.0 g, 2.88 mmol) in 30 mL THF/EtOH (1:1) was added 0.1 g of Pd/C (10%) and the contents stirred under hydrogen gas (40 psi) for 6 h. The reaction was then filtered through celite, washed with EtOH and the contents concentrated under vacuum to afford 2-[(4-phenylpiperidin-1-yl)methyl]quinolin-6-amine as a green solid (0.8g, 87%). ¹H NMR (300 MHz, DMSO-d₆) δ 7.91 (d, J=8.4 Hz, 1H), 7.66 (d, J=9.0 Hz, 1H), 7.41 (d, J=8.5 Hz, 1H), 7.39-7.10 (m, 6H), 6.78 (s, 1H), 3.45 (s, 2H), 2.94 (m, 2H), 2.52 (m, 2H), 2.16 (m, 2H), 1.74-1.65 (m, 3H); ES-LCMS m/z 320 (M+H).

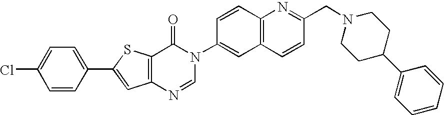

### Preparation 18

### 8-Methoxymethoxy-4-(4-methoxymethoxy-phenyl)-1,3a,4,9b-tetrahydro-3H-cyclopenta[c]chromen-2-one (23)

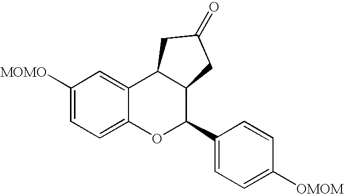

To a solution of flavan 22 (847 mg, 1.91 mmol) in 18 mL of THF was added a solution of LiOH (230 mg, 9.58 mmol) in 9 mL of water. Add 8 mL of THF and 4 mL of water. After stirring for 1 hr, NaH₂PO₄ (9.6 mL of a 1 M solution in water, 9.6 mmol) was added followed by NaIO₄ (2.0 g, 9.35 mmol). After stirring for 1 hr, the solution was diluted with EtOAc. The aqueous solution was separated and extracted with EtOAc. The combined organic solutions were washed with 1:1 saturated aqueous Na₂SO₃:bicarbonate, brine, dried over Na₂SO₄, filtered, and concentrated to give 760 mg, 1.97 mmol, 100% of cyclopentanone 23. ¹H NMR (400 MHz, CDCl₃) δ 7.35 (d, 2H, J=8.8 Hz), 7.06 (d, 2H, J=8.7 Hz), 6.90-6.81 (m, 3H), 5.19 (s, 2H), 5.14-5.08 (m, 3H), 3.87 (t, 1H, J=7.5 Hz), 3.49 (s, 3H), 3.48 (s, 3H), 2.93 (m, 1H), 2.78 (dd, 1H, J=18.5, 8.4 Hz), 2.63 (d, 1H, J=18.5 Hz), 2.33 (dd, 1H, J=18.6, 12.1 Hz), 2.04 (dd, 1H, J=18.6, 8.1 Hz).

### EXAMPLE 8

### Preparation of (3aR, 4S, 9bS)- and (3aS, 4R, 9bR)-8-Hydroxy-4-(4-hydroxy-phenyl)-1,3a,4,9b-tetrahydro-3H-cyclopenta[c]chromen-2-one

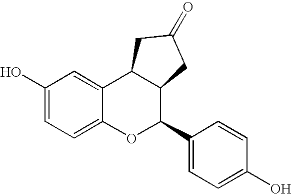

### (3aR, 4S, 9bS)- and (3aS, 4R, 9bR)-8-Hydroxy-4-(4-hydroxy-phenyl)-1,3a,4,9b-tetrahydro-3H-cyclopenta[c]chromen-2-one (24)

Stir a solution of cyclopentanone 23 (384 mg, 1.0 mmol) in 10 mL of THF and 8 mL of 3 M HCl overnight. Dilute the solution with EtOAc. Separate the aqueous solution and extract 2× with EtOAc. The combined organic solutions were washed with saturated aqueous sodium bicarbonate, brine, dried over Na₂SO₄, filtered, and concentrated to afford 304 mg of cyclopentanone 24. The material was purified by preparative chiral chromatography (Chiralpak AD, 65/35 heptane/ethanol).

Enantiomer A: HPLC (Zorbax C18 column; 10 to 100% CH₃CN/H₂O for 10 min then 100% CH₃CN for 5 min; 1 mL/min; tᵣ 8.34 min). HPLC (Chiralpak AD, 65/35 heptane/ethanol, 1 mL/min; t_{R}=4.1 min). LRMS(ES−) calcd for C₁₈H₁₅O₄: 295.10; found: 295.29 (M−H).

Enantiomer B: HPLC (Zorbax C18 column; 10 to 100% CH₃CN/H₂O for 10 min then 100% CH₃CN for 5 min; 1 mL/min; tᵣ 8.37 min). HPLC (Chiralpak AD, 65/35 heptane/ethanol, 1 mL/min; t_{R}=5.3 min). LRMS(ES−) calcd for C₁₈H₁₅O₄: 295.10; found: 295.29 (M−H).

### Preparation 19

### 8-Methoxymethoxy-4-(4-methoxymethoxy-phenyl)-1,2,3,3a,4,9b-hexahydro-cyclopenta[c]chromen-2-ol (25)

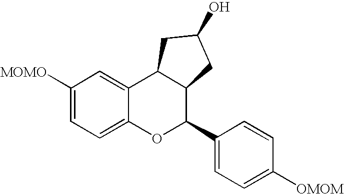

To a solution of cyclopentanon 23 (60 mg, 0.16 mmol) in 1 mL of MeOH and 0.5 mL of THF was added NaBH₄ (15 mg, 0.40 mmol). After stirring for 2 hrs saturated aqueous ammonium chloride was added. The solution was diluted with EtOAc. The aqueous solution was extracted 2× with. EtOAc. The combined organic extracts were washed with brine, dried over Na₂SO₄, filtered and concentrated to give 60 mg (0.16 mmol, 100%) of alcohol 25. HRMS (ES+) calc for C₂₂H₃₀NO₆: 404.2073, found: 404.2082 (M+NH₄).

### EXAMPLE 9

### Preparation of (2R, 3aR, 4S, 9bS)- and (2S, 3aS 4R, 9bR)-4-(4-Hydroxy-phenyl)-1,2,3,3a,4,9b-hexahydro-cyclopenta[c]chromene-2,8-diol

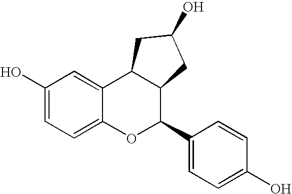

### (2R, 3aR, 4S, 9bS)- and (2S, 3aS, 4R, 9bR)-4-(4-Hydroxy-phenyl)-1,2,3,3a,4,9b-hexahydro-cyclopenta[c]chromene-2,8-diol (26)

Stir a solution of alcohol 25 (60 mg, 0.16 mmol) in 2 mL of THF and 2 mL of 3 M HCl overnight. Dilute the solution with EtOAc. Separate the aqueous solution and extract 2× with 10% MeOH in EtOAc. Wash the combined organic solutions with saturated aqueous sodium bicarbonate, brine, dry over Na₂SO₄, filter, and concentrate. Absorb to 1 g silica gel. Purify by silica gel chromatography (4 g silica gel, 0 to 10% MeOH/CH₂Cl₂ then 20% MeOH/CH₂Cl₂) to give 37 mg, (0.12 mmol, 79%) of alcohol 26. HPLC (Zorbax C18 column 10 to 100% CH₃CN/H₂O for 10 min then 100% CH₃CN for 5 min; 1 mL/min; tᵣ 7.79 min). LRMS(ES−) calcd for C₁₈H₁₅O₄: 297.11; found: 297.29 (M−H).

### Preparation 20

### 8-Methoxymethoxy-4-(4-methoxymethoxy-phenyl)-1,2,3,3a,4,9b-hexahydro-cyclopenta[c]chromen-2-ol (27)

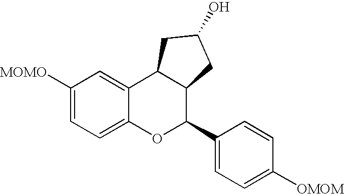

Cool a solution of alcohol 25 (50 mg, 0.13 mmol), triphenylphosphine (68 mg, 0.26 mmol), benzoic acid (24 mg, 0.2 mmol) to 0° C. Add diisopropyl azodicarboxylate (50 ul, 0.26 mmol) slowly so that temperature of reaction does not rise above about 4° C. After addition is complete, remove ice bath and warm the reaction to room temperature and stir overnight. Add MeOH to the reaction mixture and stir for 15 minutes before concentrating to a yellow oil. Purify by flash chromatography (10 g SiO₂, 40 mL/min, 0-40% EtOAc/Hexanes over 20 minutes and 40% EtOAc/Hexane for 13 minutes) to yield 67 mg of a clear oil. To a solution of the clear oil (64 mg, 0.13 mmol) in THF:H₂O (1:1, 4 mL) add lithium hydroxide (4 mg, 0.13 mmol) and stir the reaction at room temperature overnight. Heat the mixture to 60° C. with stirring for 2 hours. Cool the mixture to room temperature and neutralize with 1.0 N HCl. Dilute with EtOAc and wash with saturated sodium bicarbonate and brine. Dry the organic solution (Na₂SO₄), filter and concentrate in vacuo. Purify by flash chromatography (10 g SiO₂, 40 mL/min, 0-70% EtOAc/hexanes over 20 minutes and then 70% EtOAc/hexanes for 13 minutes) to give 41 mg (0.106 mmol, 82%) of alcohol 27 as a colorless oil. ¹H NMR (δ, 400 MHz, CDCl₃) 7.36 (d, 2H, J=8.8 Hz), 7.05 (d, 2H, J=8.8 Hz), 6.86-6.79 (m, 3H), 5.19 (s, 2H), 5.13 (d, 1H, J=6.8 Hz), 5.10 (d, 1H, 6.8 Hz), 5.07 (d, 1H, J=2.2 Hz), 4.32 (m, 1H), 3.65 (dt, 1H, J=3.5, 8.4 Hz), 3.50 (s, 3H), 3.49 (s, 3H), 2.99 (m, 1H), 2.27 (m, 1H), 2.07 (ddd, 1H, J=3.9, 5.6, 13.6), 1.87 (ddd, 1H, J=5.2, 11.6, 13.6 Hz), 1.42 (m, 1H), 1.27 (s, 1H).

HRMS(ES+) calcd for C₂₂H₃₀NO₆: 404.2073; found: 404.2057 (M+NH₄).

### Preparation 21

### 2,2-Difluoro-8-methoxymethoxy-4-(4-methoxymethoxy-phenyl)-1,2,3,3a,4,9b-hexahydro-cyclopenta[c]chromene (29)

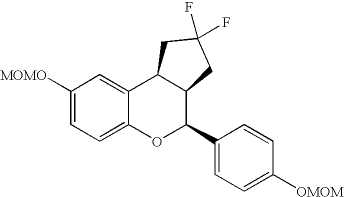

Stir a solution of cyclopentanone 23 (273 mg, 0.710 mmol) in 0.5 mL of (diethylamino)sulfur trifluoride and 0.5 mL of dichloroethane in a 4 mL vial at 40° C. overnight. Dilute with CH₂Cl₂ and wash 2× with saturated aqueous sodium bicarbonate. Dry the organic solution over Na₂SO₄, filter, and concentrate. Absorb to 5 g of silica gel and purify by silica gel chromatography (35 g silica gel, 0 to 30% EtOAc/Hexanes over 48 min at 35 mL/min) to give 217 mg (0.53 mmol, 75%) of difluorocyclopentane 29. ¹H NMR (δ, 400 MHz, CDCl₃) 7.34 (d, 2H, J=8.4 Hz), 7.06 (d, 2H, J=8.4 Hz), 6.90-6.83 (m, 2H), 6.80 (s, 1H), 5.19 (s, 2H), 5.13 (d, 1H, J=6.8 Hz), 5.11 (d, 1H, J=6.8 Hz), 5.02 (s, 1H), 3.67 (t, 1H, J=8.2 Hz), 3.49 (s, 6H), 2.89-2.67 (m, 2H), 2.40-2.09 (m, 2H), 1.88 (dt, 1H, J=14.3, 7.0 Hz).

### EXAMPLE 10

### Preparation of (3aR, 4S, 9bS)- or (3aS, 4R, 9bR)-2,2-Difluoro-4-(4-hydroxy-phenyl)-1,2,3,3a,4,9b-hexahydro-cyclopenta[c]chromen-8-ol

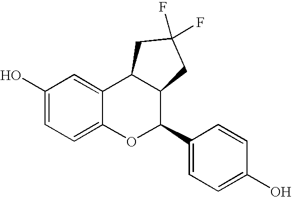

### (3aR, 4S, 9bS)- or (3aS, 4R, 9bR)-2,2-Difluoro-4-(4-hydroxy-phenyl)-1,2,3,3a,4,9b-hexahydro-cyclopenta[c]chromen-8-ol (30)

Stir a solution of difluorocyclopentane 29 (196 mg, 0.480 mmol) in 7 mL of THF and 3 mL of 3 M HCl overnight. Add 1 mL of 5 M HCl and let stir overnight. Dilute the solution with EtOAc. Separate the aqueous solution and extract 2× with EtOAc. The combined organic solutions were washed with saturated aqueous sodium bicarbonate, brine, dried over Na₂SO₄, filtered, and concentrated. Absorb to 2 g of silica gel and purify by silica gel chromatography (10 g silica gel, 10 to 60% EtOAc/Hexanes over 30 min at 35 mL/min) to give 155 mg (0.48 mmol, 100%) of difluorocyclopentane 30. The enantiomers were separated by preparative chiral chromatography (Chiralpak AD, 65/35 heptane/ethanol).

Enantiomer A: HPLC (Zorbax C18 column; 10 to 100% CH₃CN/H₂O for 10 min then 100% CH₃CN for 5 min; 1 mL/min; tᵣ 9.61 min). HPLC (Chiralpak AD, 20/80 IPA/Heptane, 1 mL/min; t_{R}=8.8 min). HRMS(CI+) calcd for C₁₈H₁₇F₂O₃: 319.1146; found: 319.1151 (M+H).

Enantiomer B: HPLC (Zorbax C18 column; 10 to 100% CH₃CN/H₂O for 10 min then 100% CH₃CN for 5 min; 1 mL/min; tᵣ 9.60 min). HPLC (Chiralpak AD, 20/80 EPA/Heptane, 1 mL/min; t_{R}=16.0 min). HRMS(CI+) calcd for C₁₈H₁₇F₂O₃: 319.1146; found: 319.1164 (M+H).

### Preparation 22

### 8-Methoxymethoxy-4-(4-methoxymethoxy-phenyl)-2-trifluoromethyl-1,2,3,3a,4,9b-hexahydro-cyclopenta[c]chromene (32 and 33)

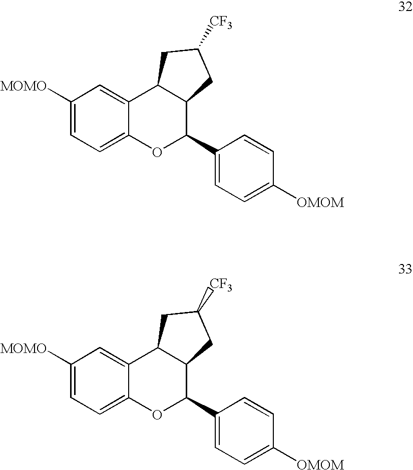

Add tetrabutylammonium fluoride (0.075 mL of a 1.0 M solution in THF, 0.075 mmol) to a solution of cyclopentanone 23 (288 mg, 0.75 mmol) and (trifluoromethyl)trimethylsilane (3.75 mL of a 0.5 M solution in THF, 1.875 mmol) in 5 mL of THF. After stirring for 2 hrs, add another 1.5 mL of (trifluoromethyl)trimethylsilane and 0.030 mL of tetrabutylammonium fluoride. After stirring for 1 hr, add another 0.75 mL of (trifluoromethyl)trimethylsilane and 0.015 mL of tetrabutylammonium fluoride. After stirring for 30 min, add saturated aqueous ammonium chloride. Extract the aqueous solution with EtOAc. Combine the organic solutions and wash with water, brine, dry over Na₂SO₄, filter and concentrate to an oil. To a solution of the oil in 5 mL of THF add TBAF (0.75 mL of a 1.0 M solution in THF, 0.075 mmol). After stirring for 15 min add saturated aqueous sodium bicarbonate. Extract the aqueous solution with EtOAc. Combine the organic solutions and wash with water, brine, dry over Na₂SO₄, filter and concentrate to 350 mg of an oil which was used without further purification. To a solution of the oil, DMAP (10 mg, 0.08 mmol) and Et₃N (0.325 mL, 2.26 mmol) in 4 mL of dichloromethane add methyl chloroglyoxylate (0.105 mL, 1.14 mmol). After stirring for 1 hr, add another 0.16 mL of Et₃N and 0.050 mL of methyl chloroglyoxylate. After stirring for 30 min dilute the solution with EtOAc, wash with saturate aqueous sodium bicarbonate, brine, dry over Na₂SO₄, filter and concentrate. Absorb to 2 g of silica gel and purify by silica gel chromatography (10 g silica gel, 0 to 30% EtOAc/Hexanes over 20 min and then 30% EtOAc/Hexanes at 35 mL/min) to give 360 mg (0.67 mmol, 89%) of an oil which was used without further purification. A solution of the oil (320 mg, 0.59 mmol), triphenylsilane (625 mg, 1.78 mmol), and AIBN (15 mg, 0.091 mmol) in 6 mL of toluene was heated to 80° C. for 4 hrs. The solution was cooled to room temperature, filtered, and the precipitate washed with Et₂O. Combine the filtrates and concentrate. Absorb to 2 g of silica gel and purify by silica gel chromatography (35 g silica gel, 0 to 30% EtOAc/Hexanes over 48 min at 35 mL/min) to give 114 mg (0.26 mmol, 44%) of trifluoromethyl 32 and 136 mg (0.31 mmol, 52%) of trifluoromethyl 33. The structures were assigned by 2D NMR spectroscopy (gDQCOSY, edited HSQC, and 2D-NOESY). Trifluoromethyl 32: HRMS(FAB) calcd for C₂₃H₂₅F₃O₅: 438.1654; found: 438.1657 (M+H). Trifluoromethyl 33: HRMS(FAB) calcd for C₂₃H₂₅F₃O₅: 438.1654; found: 438.1657 (M+H).

### EXAMPLE 11

### Preparation of (2S, 3aR, 4S, 9bS)- and (2R, 3aS, 4R, 9bR)-4-(4-Hydroxy-phenyl)-2-trifluoromethyl-1,2,3,3a,4,9b-hexahydro-cyclopenta[c]chromen-8-ol

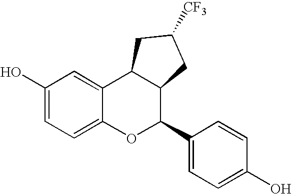

### (2S, 3aR, 4S, 9bS)- and (2R, 3aS, 4R, 9bR)-4-(4-Hydroxy-phenyl)-2-trifluoromethyl-1,2,3,3a,4,9b-hexahydro-cyclopenta[c]chromen-8-ol (34)

Stir a solution of trifluoromethyl 32 (105 mg, 0.240 mmol) in 4 mL of THF and 2 mL of 3 M HCl overnight. Add 1 mL of of THF and 0.5 mL of 12 M HCl. After stirring for 6 hrs, dilute the solution with EtOAc. Separate the aqueous solution and extract 2× with EtOAc. Wash the combined organic solutions with saturated aqueous sodium bicarbonate, brine, dry over Na₂SO₄, filter, and concentrate. Absorb to 2 g of silica gel and purify by silica gel chromatography (10 g silica gel, 0 to 40% EtOAc/Hexanes over 30 min at 35 mL/min) to give 62 mg (0.18 mmol, 74%) of trifluoromethyl 34. The enantiomers were separated by preparative chiral chromatography (Chiralpak AD, IPA/heptane).

Enantiomer A: HPLC (Zorbax C18 column; 10 to 100% CH₃CN/H₂O for 10 min then 100% CH₃CN for 5 min; 1 mL/min; tr 10.32 min). HPLC (Chiralpak AD, 30/70 IPA/Heptane, 1 mL/min; t_{R}=2.53 min). HRMS(ES−) calcd for C₁₉H₁₆F₃O₃: 349.1052; found: 349.1059 (M−H).

Enantiomer B: HPLC (Zorbax C18 column; 10 to 100% CH₃CN/H₂O for 10 min then 100% CH₃CN for 5 min; 1 mL/min; tr 10.32 min). HPLC (Chiralpak AD, 30/70 IPA/Heptane, 1 mL/min; t_{R}=3.68 min). HRMS(ES−) calcd for C₁₉H₁₆F₃O₃: 349.1052; found: 349.1078 (M−H).

### EXAMPLE 12

### Preparation of (2R, 3aR, 4S, 9bS)- and (2S, 3aS, 4R, 9bR)-4-(4-Hydroxy-phenyl)-2-trifluoromethyl-1,2,3,3a,4,9b-hexahydro-cyclopenta[c]chromen-8-ol

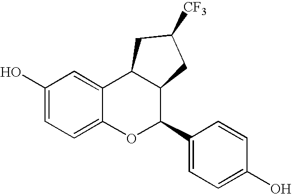

### (2R, 3aR, 4S, 9bS)- and (2S, 3aS, 4R, 9bR)-4-(4-Hydroxy-phenyl)-2-trifluoromethyl-1,2,3,3a,4,9b-hexahydro-cyclopenta[c]chromen-8-ol (35)

Stir a solution of trifluoromethyl 33 (125 mg, 0.290 mmol) in 4 mL of THF and 2 mL of 3 M HCl overnight. Add 1 mL of of THF and 0.5 mL of 12 M HCl. After stirring for 6 hrs, dilute the solution with EtOAc. Separate the aqueous solution and extract 2× with EtOAc. Wash the combined organic solutions with saturated aqueous sodium bicarbonate, brine, dry over Na₂SO₄, filter, and concentrate. Absorb to 2 g of silica gel and purify by silica gel chromatography (10 g silica gel, 0 to 50% EtOAc/Hexanes over 30 min at 35 mL/min) to give 92 mg (0.18 mmol, 91%) of trifluoromethyl 35. The enantiomers were separated by preparative chiral chromatography (Chiralpak AD, IPA/heptane).

Enantiomer A: HPLC (Zorbax C18 column; 10 to 100% CH₃CN/H₂O for 10 min then 100% CH₃CN for 5 min; 1 mL/min; tᵣ 10.13 min). HPLC (Chiralpak AD, 30/70 IPA/Heptane, 1 mL/min; t_{R}=2.96 min). HRMS(ES−) calcd for C₁₉H₁₆F₃O₃: 349.1052; found: 349.1086 (M−H).

Enantiomer B: HPLC (Zorbax C18 column; 10 to 100% CH₃CN/H₂O for 10 min then 100% CH₃CN for 5 min; 1 mL/min; tᵣ 10.13 min). HPLC (Chiralpak AD, 30/70 IPA/Heptane, 1 mL/min; t_{R}=4.66 min). HRMS(ES−) calcd for C₁₉H₁₆F₃O₃: 349.1052; found: 349.1064 (M−H).

### Preparation 23

### 2-Ethyl-8-methoxymethoxy-4-(4-methoxymethoxy-phenyl)-1,2,3,3a,4,9b-hexahydro-cyclopenta[c]chromen-2-ol (31)

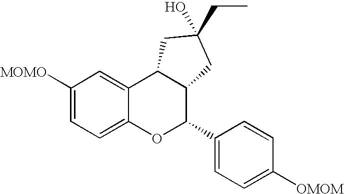

Heat CeCl₃-7H₂O (97 mg, 0.26 mmol) under vacuum at 70° C. for two hours and then warm slowly to 120° C. and continue heating overnight. Cool to room temperature and add THF (3 mL) followed by cyclopentanone 23 (100 mg, 0.26 mmol) and stir the solution for 45 minutes. Cool the reaction to −10° C., add EtMgCl (3.0 M in THF, 87 ul, 0.26 mmol) and stir the reaction for 30 minutes. Quench the reaction with saturated aqueous NH₄Cl and extract with EtOAc (2×). Combine the organic extracts, wash with brine, dry (Na₂SO₄), filter and concentrate. Purify by flash chromatography (10 g silica gel, 40 mL/min, dry loading on 700 mg of silica gel, 0-30% EtOAc/hexanes for 20 minutes and 30% EtOAc/hexanes for 13 minutes) to afford Alcohol 31 (86 mg, 0.207 mmol, 81%). ¹H NMR (δ, 400 MHz, CDCl₃) 7.36 (d, 2H, J=8.8 Hz), 7.05 (d, 2H, J=8.8 Hz), 6.90-6.87 (m, 2H), 6.83 (dd, 1H, J=8.8, 2.6 Hz), 5.19 (s, 2H), 5.14 (d, 1H, J=6.8 Hz), 5.10 (d, 1H, J=6.8 Hz), 5.05 (d, 1H, J=2.2 Hz), 3.54 (dd, 1H, J=7.6, 7.6 Hz), 3.51 (s, 3H), 3.49 (s, 3H), 2.70 (ddd, 1H, J=2.2, 7.6, 9.6 Hz), 2.22 (dd, 1H, J=13.6, 7.9 Hz), 2.03 (d, 1H, J=13.6 Hz), 1.84 (dd, 1H, J=14.1, 10.1 Hz), 1.67 (dd, 1H, J=14.1, 9.2 Hz), 1.52 (m, 2H), 0.89 (t, 3H, J=7.3 Hz).

### EXAMPLE 13

### Preparation of (21R, 3aR, 4S, 9bS)- and (2S, 3aS, 4R, 9bR)-2-Ethyl-4-(4-hydroxy-phenyl)-1,2,3,3a,4,9bR-hexahydro-cyclopenta[c]chromene-2,8-diol

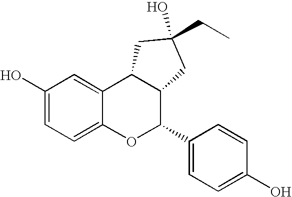

### (2R, 3aR, 4S, 9bS)- and (2S, 3aS, 4R, 9bR)-2-Ethyl-4-(4-hydroxy-phenyl)-1,2,3,3a,4,9b-hexahydro-cyclopenta[c]chromene-2,8-diol (37)

Dissolve alcohol 36 (80 mg, 0.19 mmol) in THF (2 mL) and add 3 M HCl (2.0 mL). Stir the reaction at room temperature overnight. Dilute the reaction with EtOAc and wash with saturated aqueous sodium bicarbonate and brine. Extract the aqueous solutions with EtOAc (1×). Combine organic solutions, dry (Na₂SO₄), filter and concentrate in vacuo. Purify by flash chromatography (10 g SiO₂, dry loading on 700 mg silica gel, 40 ml/min, 0-40% EtOAc/Hexane over 25 minutes and then 40% EtOAc/hexane for 7 minutes) to afford alcohol 37 (20 mg, 0.061 mmol, 32%) as a white solid. HRMS(ES+) calcd for C₂₀H₂₃O₄: 327.1596; found: 327.1596 (M+H). HPLC (Zorbax C18 column; 10 to 100% CH₃CN/H₂O for 10 min then 100% CH₃CN for 5 min; 1 mL/min; tᵣ 8.6 min).

### Preparation 24

### 2-Ethyl-8-methoxymethoxy-4-(4-methoxymethoxy-phenyl)-1,2,3,3a,4,9b-hexahydro-cyclopenta[c]chromene (38)

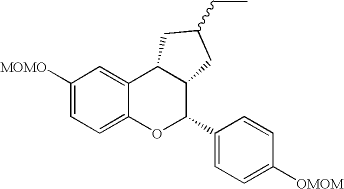

Prepare a solution of alcohol 31 (145 mg, 0.32 mmol), DMAP (5 mg, 0.035 mmol), and Et₃N (146 ul, 1.05 mmol) in CH₂Cl₂ (4 mL). Add methyl chloroglyoxylate (46 ul, 0.52 mmol) drop wise. Stir the reaction under N₂ for 30 minutes. Dilute with EtOAc and wash with saturated aqueous sodium bicarbonate, 1.0 M HCl, saturated aqueous sodium bicarbonate and brine. Dry the organic solution over Na₂SO₄, filter, and concentrate in vacuo. Purification by flash chromatography (10 g SiO₂, 40 mL/min, dry loading on 500 mg silica, 0-30% EtOAc/Hexanes for 20 minutes and then 30% EtOAc/Hexanes for 13 minutes) afforded 142 mg (0.28 mmol, 81%) of an oil which was used without further purification. Dissolve the oil (138 mg, 0.28 mmol) and triphenhyl tinhydride (290 mg, 0.83 mmol) in toluene (5 mL). Add AIBN (7 mg, 0.04 mmol) and heat the solution to 80° C. and stir for 18 hours. Filter the precipitate and wash with ether. Combine the filtrates, concentrate and purify by flash chromatography (10 g SiO₂, 40 mL/min, dry loading on 800 mg silica, 0-30% EtOAc/Hexane over 20 minutes and then 30% EtOAc/hex for 13 minutes) to afford 107 mg (0.27 mmol, 99%) of alkyl cyclopentane 38 as a 4:1 mixture of diastereomers. HRMS(ES+) calcd for C₂₄H₃₄NO₅: 416.2437; found: 416.2432 (M+NH₄).

### EXAMPLE 14

### Preparation of (2S, 3aS, 4R, 9bR)- and (2R, 3aS, 4R, 9bR)- and (2S, 3aR, 4S, 9bS)- and (2R, 3aR, 4S, 9bS)-2-Ethyl-4-(4-hydroxy-phenyl)-1,2,3,3a,4,9b-hexahydro-cyclopenta[c]chromen-8-ol

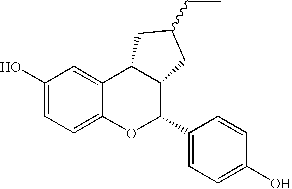

### (2S, 3aS, 4R, 9bR)- and (2R, 3aS, 4R, 9bR)- and (2S, 3aR, 4S, 9bS)- and (2R, 3aR, 4S, 9bS)-2-Ethyl-4-(4-hydroxy-phenyl)-1,2,3,3a,4,9b-hexahydro-cyclopenta[c]chromen-8-ol (39)

Dissolve alkyl cyclopentane 38 (109 mg, 0.27 mmol) in THF (4 mL) then add 3 M HCl (1.0 mL). Stir the reaction at room temperature overnight. Dilute the reaction with EtOAc and wash with saturated aqueous sodium bicarbonate and brine. Extract the aqueous layer with EtOAc (1×). Combine the organic extracts, dry (Na₂SO₄), filter and concentrate in vacuo. Purify by flash chromatography (10 g SiO₂, dry loading on 700 mg silica, 40 ml/min, 0-30% EtOAc/Hexane over 25 minutes and then 30% EtOAc/hexane for 7 minutes) to afford 56 mg (0.18 mmol, 68%) of alkyl cyclopentane 39 as a white solid. HRMS(ES+) calcd for C₂₀H₂₆NO₃: 328.1913; found: 328.1906 (M+NH₄). HPLC (Zorbax C18 column; 10 to 100% CH₃CN/H₂O for 10 min then 100% CH₃CN for 5 min; 1 mL/min; tᵣ 9.33 min).

### Preparation 25

### 2-Methoxy-8-methoxymethoxy-4-(4-methoxymethoxy-phenyl)-1,2,3,3a,4,9b-hexahydro-cyclopenta[c]chromene (40)

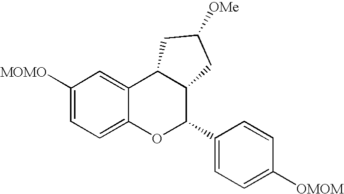

To a solution of alcohol 25 (200 mg, 0.52 mmol) in DMF (5 mL) add sodium hydride (60% dispersion in mineral oil, 21 mg, 0.51 mmol) and stir the reaction at room temperature for 10 minutes. Cool the reaction to 0° C. and add methyl iodide (33 ul, 0.52 mmol) and stir the reaction mixture for 2 hours. Quench the reaction with saturated NH₄Cl and extract with EtOAc (2×). Combine the organic extracts and wash with H₂O, saturated aqueous sodium bicarbonate and brine. Dry (Na₂SO₄), filter and concentrate the solution i)i vacuo. Purify by flash chromatography (10 g SiO₂, 40 mL/min, 0-40% EtOAc/Hexanes over 20 minutes and then 40% EtOAc/Hexanes for 13 minutes) to methyl ether 40 (210 mg, 0.52 mmol, 100%) as a yellow oil HRMS(ES+) calcd for C₂₃H₂₉O₆: 401.1964;. found: 401.1969 (M+H).

### EXAMPLE 15

### Preparation of (2S, 3aS, 4R, 9bR)- and (2R, 3aR, 4S, 9bS)-4-(4-Hydroxy-phenyl)-2-methoxy-1,2,3,3a,4,9b-hexahydro-cyclopenta[c]chromen-8-ol

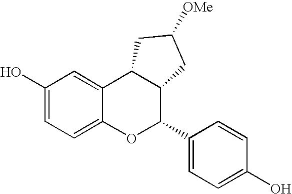

### (2S, 3aS, 4R, 9bR)- and (2R, 3aR, 4S, 9bS)-4-(4-Hydroxy-phenyl)-2-methoxy-1,2,3,3a,4,9b-hexahydro-cyclopenta[c]chromen-8-ol (41)

Dissolve methyl ether 40 (205 mg, 0.51 mmol) in THF (8 mL) and add 3M HCl (2 mL). Stir the reaction at room temperature overnight. Dilute the reaction with EtOAc and wash with saturated aqueous sodium bicarbonate and brine. Extract the aqueous solutions with EtOAc (1×). Combine the organic solutions, dry (Na₂SO₄), filter and concentrate them in vacuo. Purify the product by flash chromatography (10 g SiO₂, dry loading on 700 mg silica, 40 ml/min, 0-50% EtOAc/Hexane over 20 minutes and then 50% EtOAc/hexane for 13 minutes) to afford methyl ether 41 (125 mg, 0.4 mmol, 78%) as a white solid. HRMS(ES+) calcd for C₁₉H₂₃NO₄: 330.1705; found: 330.1695 (M+NH₄); HPLC (Zorbax C18 column; 10 to 100% CH₃CN/H₂O for 10 min then 100% CH₃CN for 5 min; 1 mL/min; tᵣ 8.95 min). The enantiomers wre separated by preparative chiral chromatography, chiralpol AD, IPA/Heptane.

Enantiomer A: HRMS(ES+) calcd for C₁₉H₂₄NO₄: 330.1705; found: 330.1691 (M+NH₄). HPLC (Chiralpak AD, 30-70% IPA/Heptane for 15 min; 1 mL/min; t_{R}=3.52 min). HPLC (Zorbax C18 column; 10 to 100% CH₃CN/H₂O for 10 min then 100% CH₃CN for 5 min; 1 mL/min; tᵣ 8.95 min).

Enantiomer B: HRMS(ES+) calcd for C₁₉H₂₄NO₄: 330.1705; found: 330.1695 (M+NH₄). HPLC (Chiralpak AD, 30-80% IPA/Heptane for 15 min; 1 mL/min; t_{R}=6.15 min). HPLC (Zorbax C18 column; 10 to 100% CH₃CN/H₂O for 10 min then 100% CH₃CN for 5 min; 1 mL/min; tᵣ 8.96 min).

### Preparation 25

### Acetic acid 8-methoxymethoxy-4-(4-methoxymethoxy-phenyl)-1,2,3,3a,4,9b-hexahydro-cyclopenta[c]chromen-2-yl ester (42)

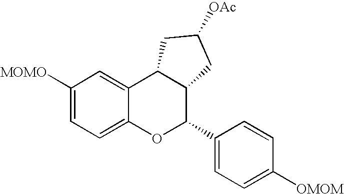

Add acetic anhydride (53 mg, 0.52 mmol) to a solution of alcohol 25 (200 mg, 0.52 mmol), Et₃N (0.14 mL, 1.03 mmol), and DMAP (6 mg, 0.052 mmol) in CH₂Cl₂ (5 mL) and stir the reaction at room temperature for 1 hour. Dilute the solution with EtOAc and wash with H₂O, saturated aqueous sodium bicarbonate and brine. Dry (Na₂SO₄), filter and concentrate the solution in vacuo. Purify the product by flash chromatography (10 g SiO₂, 40 mL/min, 0-40% EtOAc/Hexanes over 20 minutes and then 40% EtOAc/Hexanes for 13 minutes) to afford acetate 42 (183 mg, 0.43 mmol, 83%) as a yellow oil. HRMS(FAB+) calcd for C₂₄H₂₈O₇: 428.1835; found: 428.1833 (M+).

### EXAMPLE 16

### Preparation of (2S, 3aS, 4R, 9bR)- and (2R, 3aR, 4S, 9bS)-Acetic acid 8-hydroxy-4-(4-hydroxy-phenyl)-1,2,3,3a,4,9b-hexahydro-cyclopenta[c]chromen-2-yl ester

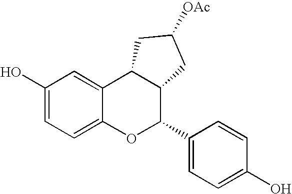

### (2S, 3aS, 4R, 9bR)- and (2R, 3aR, 4S, 9bS)-Acetic acid 8-hydroxy-4-(4-hydroxy-phenyl)-1,2,3,3a,4,9b-hexahydro-cyclopenta[c]chromen-2-yl ester (43)

Dissolve acetate 42 (180 mg, 0.42 mmol) in THF (8 mL) and add 3M HCl (2 mL). Stir the reaction at room temperature overnight. Dilute the reaction with EtOAc and wash with saturated aqueous sodium bicarbonate and brine. Extract the aqueous solutions with EtOAc (1×). Combine, dry (Na₂SO₄), filter and concentrate the organic solutions in vacuo. Purify the product by flash chromatography (10 g SiO₂, dry loading on 700 mg silica, 40 ml/min, 0-50% EtOAc/Hexane over 20 minutes and then 50% EtOAc/hexane for 13 minutes) to afford acetate 43 (47 mg, 0.14 mmol, 33%) as a white solid. The enantiomers were separated.

Enantiomer A: HRMS(ES+) calcd for C₂₀H₂₄NO₅: 358.1654; found: 358.1636 (M+NH₄). HPLC (Chiralpak AD, 20-80% IPA/Heptane for 15 min; 1 mL/min; t_{R}=3.73 min). HPLC (Zorbax C18 column; 10 to 100% CH₃CN/H₂O for 10 min then 100% CH₃CN for 5 min; 1 mL/min; tᵣ 9.07 min).

Enantiomer B: HRMS(ES+) calcd for C₂₀H₂₄NO₅: 358.1654; found: 358.1641 (M+NH₄). HPLC (Chiralpak AD, 20-80% IPA/Heptane for 15 min; 1 mL/min; t_{R}=5.35 min). HPLC (Zorbax C18 column; 10 to 100% CH₃CN/H₂O for 10 min then 100% CH₃CN for 5 min; 1 mL/min; tᵣ 9.07 min).

### Preparation 26

### 2-Fluoro-8-methoxymethoxy-4-(4-methoxymethoxy-phenyl)-1,2,3,3a,4,9b-hexahydro-cyclopenta[c]chromene (44)

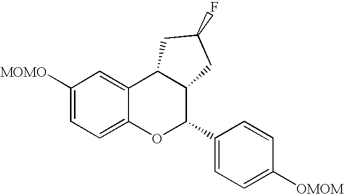

Dissolve alcohol 25 (120 mg, 0.32 mmol) in CH₂Cl₂ (5 mL). Add N,N-diethyl amino sulfurtrifluride (0.8 mL, 6.0 mmol) and stir the reaction at room temperature overnight. Dilute the reaction with CH₂Cl₂ and wash with saturated aqueous sodium bicarbonate. Extract the aqueous layer with CH₂Cl₂ (1×). Combine the organic extracts, dry (Na₂SO₄), filter and concentrate them in vacuo. Purify the product by flash chromatography (10 g SiO₂, 40 mL/min, dry loading on 800 mg silica, 10-30% EtOAc/hexane over 33 minutes) to afford flurorcyclopentane 44 (84 mg, 0.217 mmol, 70%). HRMS(ES+) calcd for C₂₂H₂₆FO₅: 389.1764; found: 489.1761 (M+H).

### EXAMPLE 17

### Preparation of (2R, 3aS, 4R, 9bR)- and (2S, 3aR, 4S, 9bS)-2-Fluoro-4-(4-hydroxy-phenyl)-1,2,3,3a,4,9b-hexahydro-cyclopenta[c]chromen-8-ol

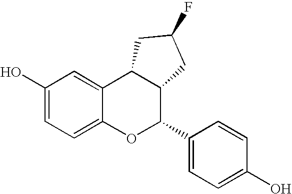

### (2R, 3aS, 4R, 9bR)- and (2S, 3aR, 4S, 9bS)-2-Fluoro-4-(4-hydroxy-phenyl)-1,2,3,3a,4,9b-hexahydro-cyclopenta[c]chromen-8-ol (45)

Dissolve fluorocyclopentane 44 (78 mg, 0.201 mmol) in THF (2 mL) and add 3M HCl (0.5 mL). Stir the reaction at room temperature overnight. Dilute the reaction with EtOAc and wash with saturated aqueous sodium bicarbonate and brine. Extract the aqueous solutions with EtOAc (1×). Combine the organic extracts, dry (Na₂SO₄), filter and concentrate them in vacuo. Purify the product by flash chromatography (10 g SiO₂, dry loading on 700 mg silica, 40 ml/min, 0-30% EtOAc/Hexane over 20 minutes and then 30% EtOAc/hexane for 13 minutes) to afford fluorocyclopentane 45 (54 mg, 0.18 mmol, 90%) as a white solid. The enantiomers were separated by preparative chiral chromatography (Chiralpak AD, IPA/heptane).

Enantiomer A: HRMS(ES+) calcd for C₁₈H₁₇FO₃: 301.1240; found: 301.1221 (M+H). HPLC (Chiralpak AD, 20-80% IPA/Heptane for 15 min; 1 mL/min; t_{R}=5.88 min). HPLC (Zorbax C18 column; 10 to 100% CH₃CN/H₂O for 10 min then 100% CH₃CN for 5 min; 1 mL/min; tᵣ 9.46 min).

Enantiomer B: HRMS(ES+) calcd for C₁₈H₁₈FO₃: 301.1240; found: 301.1226 (M+H). HPLC (Chiralpak AD, 20-80% IPA/Heptane for 15 min; 1 mL/min; t_{R}=7.13 min). HPLC (Zorbax C18 column; 10 to 100% CH₃CN/H₂O for 10 min then 100% CH₃CN for 5 min; 1 mL/min; tᵣ 9.49 min).

### EXAMPLE 18

### Preparation of (2S, 3aS, 4R, 9bR)- and (2R, 3aR, 4S, 9bS)-2-Fluoro-4-(4-hydroxy-phenyl)-1,2,3,3a,4,9b-hexahydro-cyclopenta[c]chromen-8-ol

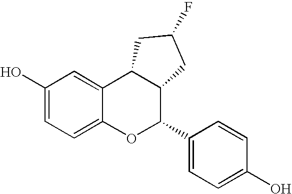

### (2S, 3aS, 4R, 9bR)- and (2R, 3aR, 4S, 9bS)-2-Fluoro-4-(4-hydroxy-phenyl)-1,2,3,3a,4,9b-hexahydro-cyclopenta[c]chromen-8-ol (47)

Fluorocyclopentane 47 was prepared from alcohol 27 in a manner substantially similar to fluorocyclopentane 45. HRMS(ES+) calcd for C₁₈H₁₈FO₃: 301.1240; found: 301.1241 (M+H).

### Preperation 27

### 6-Benzyloxy-2-oxo-2H-chromene-3-carboxylic acid ethyl ester

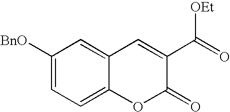

To a 0 C solution of the phenol (26.7 g, 114 mmol) and benzyl bromide (20.5 mL, 171 mmol) in DMF (300 mL) add NaH (6.84 g, 1.5 mmol) portionwise over 15 min. Allow venting during the addition, during which time the solution turns dark red. After 30 min, remove the cooling bath and allow the solution to warm to 23 C, during which time a precipitate forms and the solution turns dark brown. After 2 h, slowly pour the solution into ½ satd. NaHCO₃ (500 mL), and filter the mixture. Wash the filter cake with H₂O (2×300 mL) and 50% Et₂O/hexanes (2×300 mL) to remove remaining aqueous salts and excess benzyl bromide. Dry the remaining yellow solid to afford Preparation 27 (28.9 g, 78%). ¹H NMR (d-DMSO) δ 8.67 (s, 1 H), 7.57 (s, 1 H), 7.37-7.47 (m, 7 H), 5.15 (s, 2 H), 4.28 (q, J=7.2 Hz, 2 H), 1.31 (t, J=7.2 Hz, 3 H).

### Preparation 28

### 2-Benzyloxy-6,9-dioxo-8,9,10,10a-tetrahydro-7H-benzo[c]chromene-6a-carboxylic acid ethyl ester

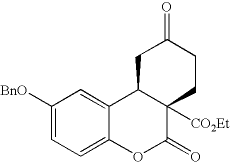

Heat a suspension of Preparation 27, (12.0 g, 37.0 mmol), 2-trimethylsilyloxybutadiene (7.1 g, 55.5 mmol) and hydroquinone (0.040 g) in o-xylenes (40 mL) to 135 C for 24 h. Allow the reaction to cool to 23 C, then pour the contents into a solution of HOAc (5 mL) in TBAF (70 mL, 1 M in THF, 70 mmol). Stir the resulting solution for 1 hour at 23 C, then slowly pour the contents into ½ satd. NaHCO₃ (150 mL) and EtOAc (250 mL). Separate the layers and wash the organic extract with brine (150 mL), dry over Na₂SO₄, and concentrate to afford a brown semisolid. Purify the product by MPLC (0 to 15 to 30% EtOAc/hexanes) to afford Preparation 28 (9.3 g, 63%) as a white solid. ¹H NMR (CDCl₃) δ 7.31-7.42 (m, 5 H), 7.04 (d, J=8.8 Hz, 1 H), 6.90 (dd, J=2.8, 8.8 Hz, 1 H), 6.73 (d, J=2.8 Hz, 1 H), 5.03 (d, 2 H), 3.98-4.16 (m, 2 H), 3.66 (dd, J=3.2, 13.2 Hz, 1 H), 2.88 (m, 1 H), 2.58-2.72 (m, 2 H), 2.49 (m, 1H), 2.38 (t, J=13.6 Hz, 1 H), 2.24 (td, J=13.6, 5.2 Hz, 1 H), 1.01 (t, J=7.2 Hz, 3 H).

### Preparation 29

### 2-Benzyloxy-7,8,10,10a-tetrahydro-6aH-benzo[c]chromene-6,9-dione

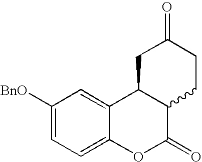

To a solution of Preparation 28 (9.25 g, 23.5 mmol) in THF (75 mL), EtOH (25 mL), and H₂O (40 mL) add lithium hydroxide hydrate (4.92 g, 117 mmol). Attach the flask to a reflux condenser and heat to 60 C for 1 h. Allow the contents to cool to 23 C and pour them into 1 N HCl and extract with Et₂O (2 x 75 mL) and EtOAc (2×75 mL). Wash the combined organic extracts with brine, dry over Na₂SO₄, and concentrate to afford the intermediate carboxylic acid as an off-white solid, which is used immediately in the next step.

Add o-xylenes (100 mL) to the flask containing the crude acid, and heat the resulting heterogeneous solution to reflux for 2 h. Concentrate the mixture via rotary evaporator to afford Preparation 29 (approx. 9 g, ˜quantitative) as an approximately 3:1 inseparable mixture of diastereomers. No further purification is required. ¹H NMR (CDCl₃) δ 7.30-7.44 (m, 5 H), 7.02 (d, J=8.8 Hz, 1 H), 6.89 (dd, J=8.8, 2.8 Hz, 1 H), 6.74 (d, J=2.8 Hz, 1 H), 5.04 (s, 3 H), 3.02-3.36 (m, 2 H), 2.54-2.77 (m, 3 H), 2.36-2.45 (m, 2 H), 1.93-2.02 (m, 1 H).

### Preparation 30

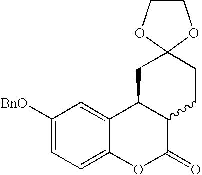

To a solution of Preparation 29 (˜9 g, ˜23 mmol) and ethylene glycol (2.79 mL, 50 mmol) in toluene (135 mL) add paratoluene sulfonic acid monohydrate (0.44 g, 2.3 mmol). Attach a Dean-Stark trap, and heat the solution to reflux for 2 h. Allow the solution to cool to 23 C, then pour the contents into ½ satd. NaHCO₃ (150 mL) and EtOAc (150 mL). Filter the mixture, and wash the filter cake with EtOAc and CH₂Cl₂. Separate the layers, and further extract the aqueous layer with EtOAc (100 mL) and CH₂Cl₂ (100 mL). Wash the combined organic extracts with brine, dry over Na₂SO₄, and concentrate. Recrystallization from hexanes/toluene (9:1) followed by MPLC purification of the mother liquors (0 to 25 to 40% EtOAc/hexanes) affords Preparation 30 as an inseparable mixture of diastereomers (7.02 g, 82% over 3 steps). Note: Purification may be considered optional, as ¹H NMR of the crude product is fairly clean. ¹H NMR (CDCl₃) δ 7.30-7.44 (m, 5 H), 6.95 (d, J=8.8 Hz, 1 H), 6.84 (dd, J=8.8, 2.8 Hz, 1 H), 6.78 (d, J=2.8 Hz, 1 H), 5.03 (s, 2 H), 3.98 (m, 4 H), 3.02-3.24 (m, 1 H), 2.30-2.90 (m, 2 H), 1.90-2.22 (m, 4 H), 1.54-1.67 (m, 1 H).

### Preparation 31

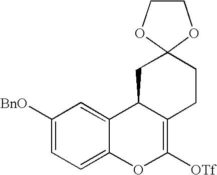

To a −78 C solution of Preparation 30 (7.0 g, 19.1 mmol) in THF (80 mL) add potassium hexamethyldisilane (KHMDS) (53 mL, 0.5 M solution in toluene, 26.7 mmol) over 5 min. Add hexamethylphosphoramide (HMPA) (4.64 mL, 26.7 mmol) quickly, and stir the solution at −78 C for 25 min. Add a solution of N-phenyl triflamide (11.5 g, 32.2 mmol) in THF (15 mL+rinse) via syringe. Maintain the resulting solution at −78 C for 2 h, then pour the reaction contents into ½ satd. NaHCO₃ and extract with Et₂O (150 mL) and EtOAc (2×75 mL). Wash the combined organic extracts with H₂O (2×100 mL) and brine (100 mL), dry over Na₂SO₄, and concentrate. Purification of the crude product by MPLC (0 to 12 to 25% EtOAc/hexanes) affords Preparation 31 (6.25 g, 66%) as an off-white solid. ¹H NMR (CDCl₃) δ 7.25-7.43 (m, 5 H), 6.84 (d, J=8.8 Hz, 1 H), 6.80 (dd, J=8.8, 2.8 Hz, 1 H), 6.72 (d, J=2.8 Hz, 1 H), 5.02 (s, 2 H), 4.04 (m, 4 H), 3.82 (dd, J=4.4, 12.8 Hz, 1 H), 2.66 (dq, J=14.0, 2.4 Hz, 1 H), 2.21 (m, 2 H), 1.91 (m, 1H), 1.80 (t, J 12.8 Hz, 1 H), 1.64 (td, J=12.8, 4.4 Hz, 1 H).

### Preparation 32

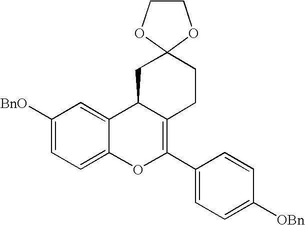

Sparge N₂ (g) through a solution of Preparation 31 (3.0 g, 6.0 mmol), p-benzyloxyphenylboronic acid (1.65 g, 9.0 mmol), and LiCl (0.77 g, 18.1 mmol) in DME (40 mL) and aqueous Na₂CO₃ (7.5 mL, 2 M in H₂O, 15 mmol) for 15 min. Add palladium tetrakis triphenylphosphine (0.69 g, 0.60 mmol), then heat the solution to reflux for 24 h, during which time the product precipitates out as a white solid. Allow the solution to cool to 23 C, then pour the contents into ½ satd NaHCO3/ Et₂O and filter. Wash the filter cake with H₂O and cold Et₂O, affording 2.0 g of Preparation 32. Extract the filtrate with EtOAc (3×50 mL) and dry the combined organic extracts over Na₂SO₄ and concentrate to afford the remaining crude product. Purification of the crude material by silica gel chromatography (CH₂Cl₂) affords another 1.04 g of Preparation 32. The total yield is 3.04 g (95%). ¹H NMR (CDCl₃) δ 7.31-7.47 (m, 12 H), 7.01 (d, J=8.8 Hz, 2 H), 6.84 (d, J=8.8 Hz, 1 H), 6.77 (d, J=8.8 Hz, 2 H), 5.11 (s, 2 H), 5.03 (s, 2 H), 3.98-4.12 (m, 4 H), 3.74 (dd, J=12.8, 4.2 Hz, 1 H), 2.58 (m, 1 H), 2.24 (m, 1 H), 2.14 (td, J=12.8, 4.2 Hz, 1 H), 1.85 (t, J=12.8 Hz, 2 H), 1.58 (m, 1 H).

### Preparation 33

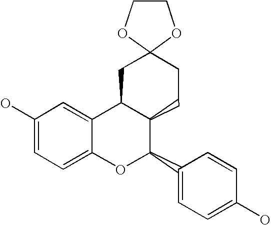

To a mixture of 10 wt % Pd on carbon (0.5 g) in MeOH (100 mL) add a slightly soluble solution of Preparation 32 (3.0 g, 5.63 mmol) in THF (25 mL). Heat the solution to 40 C and maintain under 60 psi of H₂ (g) for 4 h. Filter the solution and concentrate the filtrate to afford Preparation 33 (˜1.8 g crude, ˜quantitative) as a white solid. ¹H NMR (CD₃OD) δ 7.22 (d, J=8.8 Hz, 2 H), 6.77 (m, 3 H), 6.66 (d, J=8.8 Hz, 1 H), 6.53 (dd, J=3.2, 8.8 Hz, 1 H), 4.98 (s, 1 H), 3.88 (m, 1 H), 3.76 (m, 2 H), 3.68 (m, 1 H), 3.47 (m, 1 H), 2.48 (d, J=14.8 Hz, 1 H), 1.99 (m, 1 H), 1.88 (dd, J=14.8, 6.0 Hz, 1 H), 1.49-1.61 (m, 2 H), 1.37-1.46 (m, 1 H), 1.27 (m, 1 H), 1.17 (t, J=7.2 Hz, 3 H).

### EXAMPLE 19

### Preparation of (6S, 6aR, 10aS)-2-Hydroxy-6-(4-hydroxy-phenyl)-6,6a,7,8,10,10a-hexahydro-benzo[c]chromen-9-one

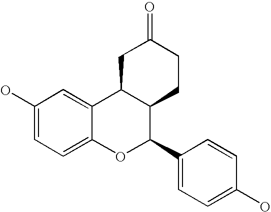

### (6S, 6aR, 10aS)-2-Hydroxy-6-(4-hydroxy-phenyl)-6,6a,7,8,10,10a-hexahydro-benzo[c]chromen-9-one

To a solution of Preparation 33 (˜1.7 g crude) in THF (40 mL) and H₂O (1 mL) add HCl solution (6 mL, 3 N in H₂O), and stir the mixture overnight. Pour the mixture into satd. NaHCO₃ and extract with Et₂O (2×50 mL) and EtOAc (2×50 mL). Wash the combined organic extracts with brine, dry over Na₂SO₄, and concentrate to afford Example 19 (˜1.3 g, ˜quantitative) as a light yellow solid. This material is of suitable purity to be used crude for analogue development, but can be recrytallized from a variety of solvents (toluene/MeOH/hexanes or iPrOH/hexanes) for characterization. ¹H NMR (CD₃OD) δ 7.31 (d, J=8.8 Hz, 2 H), 6.85 (d, J=8.4 Hz, 2 H), 6.75 (d, J=8.4 Hz, 2 H), 6.62 (dd, J=8.8, 2.4 Hz, 1 H), 5.25 (s, 1 H), 3.89 (m, 1 H), 2.98 (m, 2 H), 2.58 (m, 1 H), 2.38 (m, 1 H), 2.13 (br d, J=14.4 Hz, 1 H), 1.66 (m, 2 H).

### Preparation 34

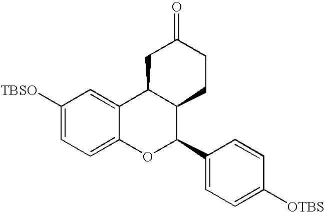

### 2-(tert-Butyl-dimethyl-silanyloxy)-6-[4-(tert-butyl-dimethyl-silanyloxy)-phenyl]-6,6a,7,8,10,10a-hexahydro-benzo[c]chromen-9-one

To a solution of Example 19 (0.120 g, 0.39 mmol) and imidazole (0.079 g, 1.16 mmoL) in DMF (2.5 mL) add tert-butyldimethylsilyl chloride (0.131 g, 0.87 mmol). Allow the reaction to stir for 1 h, then pour into ½ satd. NaHCO₃ (50 mL) and extract with Et₂O (2×25 mL) and EtOAc (25 mL). Wash the combined organic extracts with H₂O (2×25 mL) and brine (25 mL), and dry the organics over Na₂SO₄. Concentrate the mixture, and purify the residue by MPLC (0% to 10% to 20% EtOAc/hexanes) to afford Preparation 34 (0.184 g, 88%) as a white solid. ¹H NMR (CDCl₃) δ 7.30 (d, J=8.4 Hz, 2 H), 6.88 (d, J=8.4 Hz, 2 H), 6.77 (d, J=8.8 Hz, 1 H), 6.74 (d, J=2.8 Hz, 1 H), 6.63 (dd, J=8.8, 2.8 Hz, 1 H), 5.27 (s, 1 H), 3.85 (m, 1 H), 3.01 (d, J=15.2 Hz, 1 H), 2.79 (dd, J=5.8, 15.2 Hz, 1 H), 2.45 (m, 1 H), 2.22 (m, 2 H), 1.60-1.80 (m, 2 H), 1.01 (s, 9 H), 0.99 (s, 9 H), 0.23 (s, 6 H), 0.21 (s, 3 H), 0.19 (s, 3 H).

### Preparation 35

### 2-Methoxymethoxy-6-(4-methoxymethoxy-phenyl)-6,6a,7,8,10,10a-hexahydro-benzo[c]chromen-9-one

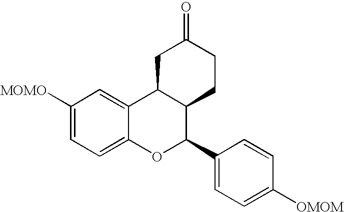

To a 0 C solution of Example 19 (0.100 g, 0.32 mmol) in THF (3 mL) add potassium tert-butoxide (0.090 g, 0.81 mmol) followed by methoxymethyl chloride (MOM-Cl) (0.061 mL, 0.81 mmol). Remove the ice bath and stir for 1 h at room temperature. Pour the contents into ½ satd. NaHCO₃ (50 mL) and extract with Et₂O (2×25 mL) and EtOAc (2×25 mL). Wash the combine organic extracts with brine (50 mL), dry over Na₂SO₄, and concentrate to afford a brown residue. Purify the residue by MPLC (0% to 25% to 50% EtOAc/hexanes) to afford Preparation 35 (0.102 g, 80%). ¹H NMR (CDCl₃) δ 7.37 (d, J=8.4 Hz, 2 H), 7.09 (d, J=8.4 Hz, 2 H), 6.96 (d, J=2.4 Hz, 1 H), 6.87 (dd, J=2.4, 8.8 Hz, 1 H), 6.84 (d, J=8.8 Hz, 1 H), 5.28 (s, 1 H), 5.20 (s, 2 H), 5.13 (A of AB, J_{AB}=7.0 Hz, 1 H), 5.07 (B of AB, J_{AB}=7.0 Hz, 1 H), 3.87 (m, 1 H), 3.50 (s, 3 H), 3.48 (s, 3 H), 3.04 (br d, J=14.4 Hz, 1 H), 2.79 (dd, J=6.2, 14.4 Hz, 1 H), 2.46 (m, 1 H), 2.21 (m, 2 H), 1.62-1.79 (m, 2 H).

### Preparation 36

### 2-(tert-Butyl-dimethyl-silanyloxy)-6-[4-(tert-butyl-dimethyl-silanyloxy)-phenyl]-9-methylene-6a,7,8,9,10,10a-hexahydro-6H-benzo[c]chromene

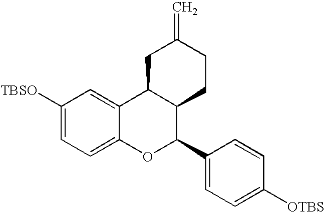

To a −40 C solution of Preparation 34 (0.100 g, 0.19 mmol) in THF (2 mL) and pyridine (0.045 mL) add the Tebbe reagent (Cp₂ZrCl(H)Me) (0.74 mL, 0.5 M toluene, 0.37 mmol). Maintain the reaction at −40 C for 1 h, then pour the contents into ½ satd. NaHCO₃ (50 mL) and extract with Et₂O (2×25 mL) and EtOAc (2×25 mL). Wash the combine organic extracts with brine (50 mL), dry over Na₂SO₄, and concentrate to afford a brown residue. Purify the residue by MPLC (0% to 5% to 10% EtOAc/hexanes) to afford Preparation 36 (0.093 g, 93%). ¹H NMR (CDCl₃) δ 7.27 (d, J=8.4 Hz, 2 H), 6.85 (d, J=8.4 Hz, 2 H), 6.75 (m, 2 H), 6.61 (dd, J=2.4, 8.8 Hz, 1 H), 5.15 (s, 1 H), 4.62 (m, 2 H), 3.49 (br s, 1 H), 2.91 (d, J=14.4 Hz, 1 H), 2.51 (dd, J=14.4, 4.6 Hz, 1 H), 2.15 (m, 2 H), 1.92 (td, J=12.8, 5.6 Hz, 1 H), 1.33 (m, 2 H), 1.01 (s, 18 H), 0.22 (s, 6 H), 0.19 (s, 3 H), 0.18 (s, 3 H).

### EXAMPLE 20

### Preparation of (6aR, 6S, 10aS)-6-(4-Hydroxy-phenyl)-9-methylene-6a,7,8,9,10,10a-hexahydro-6H-benzo[c]chromen-2-ol

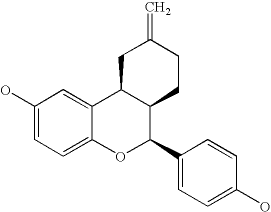

### (6aR, 6S, 10aS)-6-(4-Hydroxy-phenyl)-9-methylene-6a,7,8,9,10,10a-hexahydro-6H-benzo[c]chromen-2-ol

To a 0 C solution of Preparation 36 (0.093 g, 0.17 mmol) in THF (5 mL) add a solution of tetra-n-butyl ammonium fluoride (0.43 mL, 1 M in THF, 0.43 mmol). Stir the solution at 0 C for 1 h, then pour the contents into ½ satd. NaHCO₃ (50 mL) and extract with Et₂O (2×25 mL) and EtOAc (2×25 mL). Wash the combine organic extracts with brine (50 mL), dry over Na₂SO₄, and concentrate to afford a brown residue. Purify the residue by MPLC (0% to 25% to 40% EtOAc/hexanes) to afford Example 22 (0.028 g, 52%) as a white solid. ¹H NMR (CD₃OD) δ 7.26 (d, J=8.8 Hz, 2 H), 6.82 (d, J=8.8 Hz, 2 H), 6.79 (d, J=3.2 Hz, 1 H), 6.69 (d, J=8.4 Hz, 1 H), 6.57 (dd, J=3.2, 8.4 Hz, 1 H), 5.09 (s, 1 H), 4.62 (m, 1 H), 3.48 (s, 1 H), 2.97 (d, J=13.6 Hz, 1 H), 2.54 (dd, J=5.2, 13.6 Hz, 1 H), 2.17 (m, 2 H), 1.95 (td, J=5.2, 12.8 Hz, 1 H), 1.25-1.38 (m, 2 H).

### EXAMPLE 21

### Preparation of (6aR, 6S, 9S, 10aS)-6-(4-Hydroxy-phenyl)-9-methyl-6a,7,8,9,10,10a-hexahydro-6H-benzo[c]chromen-2-ol and (6aR, 6S, 9R, 10aS)-6-(4-Hydroxy-phenyl)-9-methyl-6a,7,8,9,10,10a-hexahydro-6H-benzo[c]chromen-2-ol

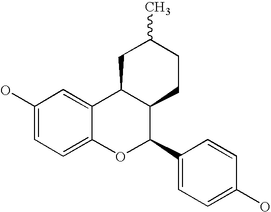

### (6aR, 6S, 9S, 10aS)-6-(4-Hydroxy-phenyl)-9-methyl-6a,7,8,9,10,10a-hexahydro-6H-benzo[c]chromen-2-ol and (6aR, 6S, 9R, 10aS)-6-(4-Hydroxy-phenyl)-9-methyl-6a,7,8,9,10,10a-hexahydro-6H-benzo[c]chromen-2-ol

To a mixture of 10 wt % Pd on carbon (0.03 g) in MeOH (20 mL) add a solution of Example 22 (0.022 g, 0.07 mmol) in MeOH (2 mL). Maintain the solution under 60 psi of H₂ (g) for 4 h. Filter the solution and concentrate the filtrate to afford Example 23 (0.022 g crude, 100%) as 3:1 ratio of epimers as a white solid. Major diastereomer: ¹H NMR (CD₃OD) δ 7.22 (d, J=8.4 Hz, 2 H), 6.81 (d, J=2.4 Hz, 1 H), 6.78 (d, J=8.4 Hz, 2 H), 6.68 (d, J=8.8 Hz, 1 H), 6.54 (dd, J=8.8, 2.4 Hz, 1 H), 4.94 (s, 1 H), 3.30 (m, 1 H), 2.23 (d, J=13.6 Hz, 1 H), 1.95 (m, 2 H), 1.33-1.56 (m, 3 H), 1.20 (m, 1 H), 1.11 (m, 1 H), 0.63 (d, J=7.2 Hz, 3 H).

### Preparation 37

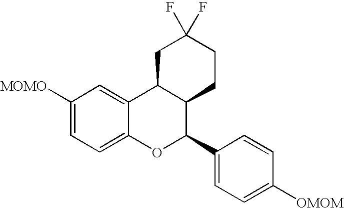

### 9,9-Difluoro-2-methoxymethoxy-6-(4-methoxymethoxy-phenyl)-6a,7,8,9,10,10a-hexahydro-6H-benzo[c]chromene

Heat a mixture of Preparation 35 (0.102 g, 0.26 mmol) and (Diethylamino) sulfur trifluoride (0.25 mL) in 1,2-dichloroethane (0.75 mL) to 40 C for 12 h. Purify the mixture by MPLC (0% to 10% to 25% EtOAc/hexanes) to afford Preparation 37 (0.042 g, 39%). ¹H NMR (CDCl₃) δ 7.35 (d, J=8.8 Hz, 2 H), 7.08 (d, J=8.8 Hz, 2 H), 7.02 (s, 1 H), 6.86 (m, 2 H), 5.20 (s, 2 H), 5.16 (s, 1 H), 5.15 (A of AB, J_{AB}=6.4 Hz, 1 H), 5.11 (B of AB, J_{AB}=6.4 Hz, 1 H), 3.66 (br s, 1 H), 3.50 (s, 6 H), 2.84 (m, 1 H), 1.96-2.23 (m, 3 H), 1.54-1.69 (m, 2 H), 1.44 (m, 1 H).

### EXAMPLE 22

### Preparation of (6aR, 6S, 10aS)-9,9-Difluoro-6-(4-hydroxy-phenyl)-6a,7,8,9,10,10a-hexahydro-6H-benzo[c]chromen-2-ol

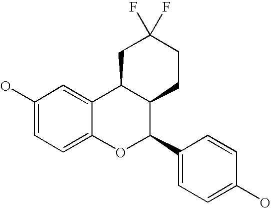

### (6aR, 6S, 10aS)-9,9-Difluoro-6-(4-hydroxy-phenyl)-6a,7,8,9,10,10a-hexahydro-6H-benzo[c]chromen-2-ol

Add a solution of HCl (2 mL, 3 N in H₂O) to Preparation 37 (0.042 g, 0.10 mmol) in THF (5 mL) and H₂O (1 mL) and stir the mixture for 12 h. Pour the mixture into satd. NaHCO₃ and extract with Et₂O (2×50 mL) and EtOAc (2×50 mL). Wash the combined organic extracts with brine, dry over Na₂SO₄, and concentrate to afford the desired product as a light yellow solid. Purify the crude material by MPLC (0% to 25% to 40% EtOAc/hexanes) to afford Example 24 (0.014 g, 37%) as a yellow oil. ¹H NMR (CD₃OD) d 7.25 (d, J=8.4 Hz, 2 H), 6.80 (d, J=8.4 Hz, 2 H), 6.79 (d, J=2.4 Hz, 1 H), 6.69 (d, J=8.4 Hz, 1 H), 6.56 (dd, J=2.4 Hz, 1 H), 5.07 (s, 1 H), 3.62 (br s, 1 H), 2.77 (m, 1 H), 2.09-2.28 (m, 2 H), 1.90 (m, 1 H), 1.47-1.65 (m, 2 H), 1.36 (m, 1 H).

### Preparation 38

### 4-Allyl-6-benzyloxy-2-oxo-chroman-3-carboxylic acid ethyl ester

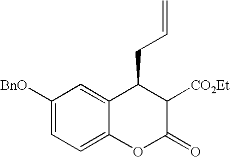

To a 0 C solution of Preparation 27 (10.0 g, 30.8 mmol) in THF (125 mL) add a solution of allyl magnesium chloride in Et₂O (46 mL, 1.0 M, 46 mmol). Maintain the reaction at 0 C for 30 min, then pour the reaction contents into a solution of ½ satd. NaHCO₃ (250 mL). Extract the solution with Et₂O (2×150 ML) and EtOAc (150 mL). Wash the combined organic extracts with H₂O (150 mL) and brine (150 mL), dry the organics over Na₂SO₄, and concentrate to afford a brown oil. Purify the product by MPLC (0% to 15% to 25% EtOAc/hexanes) to afford Preparation 38 (7.72 g, 68%) as a light yellow solid. ¹H NMR (CDCl₃) δ 7.31-7.43 (m, 5 H), 7.01 (d, J=8.8 Hz, 1 H), 6.87 (dd, J=8.8, 3.0 Hz, 1 H), 6.79 (d, J=3.0 Hz, 1 H), 5.71 (m, 1 H), 5.15 (dd, J=0.8, 9.8 Hz, 1 H), 5.10 (dd, J=0.8, 17.6 Hz, 1 H), 5.04 (A of AB, J_{AB}=14.2 Hz, 1 H), 5.03 (B of AB, J_{AB}=14.2 Hz, 1 H), 4.08 (m, 2 H), 3.80 (d, J=2.4 Hz, 1 H), 3.41 (m, 1 H), 2.35 (m, 2H), 1.08(t, J=7.2 Hz,3 H).

### Preparation 39

### 4-Allyl-6-benzyloxy-chroman-2-one

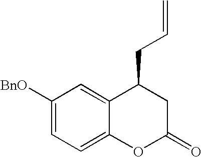

Heat a solution of Preparation 38 (4.8 g, 13.1 mmol) and LiOH (6 g) in a solution of THF (75 mL), EtOH (30 mL), MeOH (20 mL), and H₂O (50 mL) to 60 C for 2 h. Pour the contents into 1 N HCl (250 mL) and extract the mixture with Et₂O (2×200 mL) and EtOAc (2×150 mL). Wash the combined organic extracts with brine (200 mL), dry over Na₂SO₄, and concentrate to afford the crude β-keto acid.

Heat a solution of the crude acid in o-xylenes to reflux for 1.5 h. Remove the solvent in vacuo, and purify the lactone by MPLC (0% to 12% to 20% EtOAc/hexanes) to afford Preparation 39 (3.5 g, 91%) as a white solid. ¹H NMR (CDCl₃) δ 7.31-7.44 (m, 5 H), 6.99 (d, J=8.8 Hz, 1 H), 6.86 (dd, J=8.8, 3.2 Hz, 1 H), 6.81 (d, J=3.2Hz, 1 H), 5.72 (m, 1 H), 5.07-5.14 (m, 2 H), 5.05 (s, 2 H), 3.03 (m, 1 H), 2.76 (t, J=8.8 Hz, 2 H), 2.43 (m, 1 H), 2.30 (m, 1 H).

### Preparation 40

### 4-Allyl-6-benzyloxy-3-(2-methoxymethoxy-allyl)-chroman-2-one

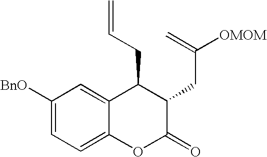

Cool a solution of Preparation 39 (3.65 g, 12.4 mmol) in THF (90 mL) to −78 C. Add a solution of KHMDS (32 mL, 0.5 M in toluene, 16 mmoL) over 5 min, then allow to stir for 15 in at −78 C. Add hexamethylphosphoramide (HMPA) via syringe (2.8 mL, 16.1 mmoL) quickly, and allow to stir for 20 min at −78 C. Add 2-O-methoxylmethyl allyl iodide (4.24 g) over 2 min, and then allow the solution to warm to −50 C over 1.5 h. Pour the contents of the reaction into ½ satd. NaHCO₃ and extract with Et₂O (2×100 mL) and EtOAc (2×100 mL). Wash the combined organic extracts with H₂O (2×150 mL) and brine (150 mL) and then dry over Na₂SO₄. Concentrate the crude product to leave a brown oil, which is purified by MPLC (0% to 12% to 20% EtOAc/hexanes) to afford Preparation 40 (3.72 g, 76%) as a light yellow oil. ¹H NMR (CDCl₃) δ 7.31-7.44 (m, 5 H), 6.98 (d, J=8.8 Hz, 1 H), 6.87 (dd, J=8.8, 3.2 Hz, 1 H), 6.74 (d, J=3.2 Hz, 1 H), 5.66 (m, 1 H), 5.01-5.09 (m, 4 H), 4.93 (A ob AB, J_{AB}=6.4 Hz, 1 H), 4.90 (B of AB, J_{AB}=6.4 Hz, 1 H), 4.18 (d, J=2.4 Hz, 1 H), 3.87 (d, J=2.4 Hz, 1 H), 3.44 (s, 3 H), 3.18 (m, 1 H), 2.86 (m, 1 H), 2.22-2.39 (m, 3 H), 2.12 (dd, J=9.4, 14.0 Hz, 1 H).

### Preparation 41

### 2-Benzyloxy-8-methoxymethoxy-6a,7,10,10a-tetrahydro-benzo[c]chromen-6-one

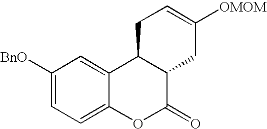

Bubble N2 gas through a solution of Preparation 40 (1.0 g, 2.54 mmol) in CH₂Cl₂ (250 mL) equipped with a reflux condenser for 30 min. Add [1,3-bis-(2,4,6-trimethylphenyl)-2-imidazolidinylidene)dichloro(phenylmethylene)-tricyclohexylphosphine)ruthenium] (0.212 g, 0.25 mmol) and heat the reaction to reflux for 2.5 h. Allow the reaction to cool to room temperature, remove the condenser, and bubble air through the mixture for 10 min. Remove the solvent in vacuo, and purify the residue by MPLC (0% to 12% to 25% EtOAc/hexanes) to afford Preparation 41 (0.72 gm, 78%) as a clear oil. ¹H NMR (CDCl₃) δ 7.32-7.45 (m, 5 H), 7.00 (d, J=8.4 Hz, 1 H), 6.86 (m, 2 H), 5.05 (m, 3 H), 5.00 (A of AB, J_{AB}=6.4 Hz, 1 H), 4.97 (B of AB, J_{AB}=6.4 Hz, 1 H), 3.45 (s, 3 H), 2.94 (m, 1 H), 2.83 (m, 1 H), 2.56-2.70 (m, 3 H), 2.25 (m, 1 H).

### Preparation 42

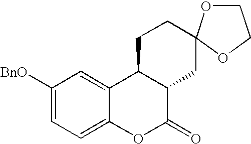

Treat a solution of Preparation 41 (0.72 g, 1.97 mmol) in THF (40 mL) with 3 N HCl (3 mL) for 4 h. Pour the contents into H₂O and extract with Et₂O and EtOAc. Wash 25 the combined organic extracts with satd. NaHCO₃ and brine, dry the combined extracts over Na₂SO₄, and concentrate to afford the crude intermediate ketone. Dissolve the ketone in toluene (40 mL) and add p-toluenesulfonic acid monohydrate (0.038 g), then attach a Dean Stark apparatus and heat the reaction to reflux for 2.5 h. Pour the contents into ½ satd. NaHCO₃ (50 mL) and separate the layers. Further extract the aqueous layer with Et2O and EtOAc (50 mL each). Wash the combined organic extracts with brine (50 mL), dry the combined organics over Na2SO4, and concentrate to afford Preparation 42 (0.74 g, 100%) as a white solid. ¹H NMR (CDCl₃) δ 7.31-7.44 (m, 5 H), 6.98 (d, J=8.4 Hz, 1 H), 6.86 (m, 2 H), 5.04 (s, 2 H), 4.03 (m, 2 H), 3.94 (m, 2 H), 2.71 (m, 1 H), 2.55 (m, 1 H), 2.35-2.46 (m, 2 H), 1.92 (m, 1 H), 1.67-1.79 (m, 3 H).

### Preparation 43

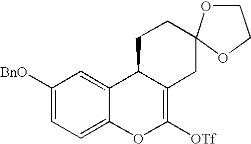

To a −78 C solution of Preparation 42 (0.366 g, 1.0 mmol) in THF (8 mL) add a solution of LDA (1.13 mL, 1.5 M cyclohexane, 1.7 mmol). Stir at −78 C for 15 min, then add HMPA (0.59 mL, 3.4 mmoL) and warm to −50 C. Stir for 15 min, then recool the solution to −78 C. Add a solution of N-phenyl triflamide (0.607 g, 1.7 mmol) in THF (2 mL) dropwise, and stir the resulting solution for 30 min. Pour the reaction contents into ½ satd. NaHCO₃, and extract the mixture with Et₂O (2×30 mL) and EtOAc (40 mL). Wash the combined organic extracts with H₂O (2×50 mL) and brine (50 mL), dry the organic layer over Na₂SO₄, and concentrate to afford the crude product. Purify the material by MPLC (0% to 15% to 25% EtOAc/hexanes) to afford Preparation 43 (0.059 g, 12%) as a yellow oil. ¹H NMR (CDCl₃) δ 7.28-7.43 (m, 5 H), 6.81 (m, 2 H), 6.75 (d, J=2.4 Hz, 1 H), 5.02 (s, 2 H), 3.99 (m, 4 H), 3.51 (q, J=5.2 Hz, 1 H), 2.73 (dd, J=2.8, 14.0 Hz, 1 H), 2.17 (m, 2 H), 1.83-1.97 (m, 3 H).

### Preparation 44

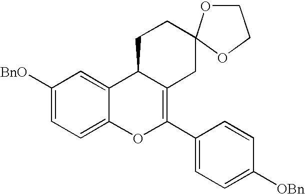

Sparge N₂ (g) through a solution of Preparation 43 (0.059 g, 0.12 mmol), p-benzyloxyphenylboronic acid (0.038 g, 0.165 mmol), and LiCl (0.025 g, 0.60 mmol) in DME (2.5 mL) and aqueous Na₂CO₃ (0.25 mL, 2 M in H₂O, 0.5 mmol) for 15 min. Add palladium tetrakis triphenylphosphine (0.035 g, 0.03 mmoL) and heat the solution to reflux for 24 h. Allow the solution to cool to 23 C, then pour the reaction contents into ½ satd NaHCO₃, and extract with EtOAc (3×25 mL). Combine the organic extracts and wash with brine (25 mL), then dry over Na₂SO₄ and concentrate. Purify the residue by MPLC (0% to 12% to 25% EtOAc/hexanes) to afford Preparation 44 (0.024 g, 38%) as a clear oil. ¹H NMR (CDCl₃) δ 7.31-7.47 (m, 10 H), 7.01 (d, J=8.8 Hz, 2 H), 6.86 (d, J=8.8 Hz, 2 H), 6.79 (s, 1 H), 6.75 (d, J=8.8 Hz, 2 H), 5.11 (s, 2 H), 5.04 (s, 2 H), 3.97 (m, 4 H), 3.43 (m, 1 H), 2.64 (dd, J=2.8, 14.0 Hz, 1 H), 2.20 (m, 1 H), 2.13 (m, 1 H), 1.99 (m, 1 H), 1.91 (m, 2 H).

### Preparation 45

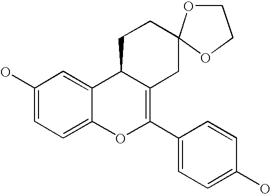

To a mixture of 10 wt % Pd on carbon (0.02 g) in MeOH (25 mL) add a solution of Preparation 44 (0.020 g, 0.04 mmol) in THF (10 mL). Maintain the solution under 60 psi of H₂ (g) for 4 h. Filter the solution and concentrate the filtrate to afford Preparation 45 (0.012 g crude, ˜quantitative) as a white solid. TLC R_{f} 0.4, 60% EtOAc/hexanes.

### EXAMPLE 23

### Preparation of (6aR, 6S, 10aS)-2-Hydroxy-6-(4-hydroxy-phenyl)-6a,9,10,10a-tetrahydro-6H,7H-benzo[c]chromen-8-one

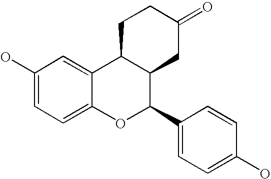

### (6aR, 6S, 10aS)-2-Hydroxy-6-(4-hydroxy-phenyl)-6a,9,10,10a-tetrahydro-6H,7H-benzo[c]chromen-8-one

To a solution of Preparation 45 (0.012 g) in THF (20 mL) and H₂O (1 mL) add HCl solution (2 mL, 3 N in H₂O), and stir the mixture overnight. Pour the mixture into satd. NaHCO₃ and extract with Et₂O (2×50 mL) and EtOAc (2×50 mL). Wash the combined organic extracts with brine, dry over Na₂SO₄, and concentrate to afford crude Preparation 27 as a light yellow solid. Purify the crude material by MPLC (0% to 25% to 50% EtOAc/hexanes) to afford Example 25 (0.010 g, 90%) as a white solid. ¹H NMR (CD₃OD) δ 7.22 (d, J=8.0 Hz, 2 H), 6.85 (m, 1 H), 6.79 (m, 3 H), 6.64 (m, 1 H), 5.22 (s, 1 H), 3.54 (m, 1 H), 2.67 (m, 1 H), 2.56 (m, 1 H), 2.26 (m, 2 H), 2.13 (m, 2 H), 1.84 (dd, J=3.9, 14.5 Hz, 1 H).

### Preparation 46

### 6-Benzyloxy-chromen-2-one

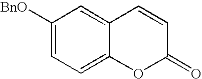

Equip a 5-L, three-neck, round-bottom flask with a large blade mechanical stirrer, thermocouple, an addition funnel, Claisen adapter, reflux condenser, and a sodium hydroxide scrubber. Charge the flask with 2,5-dimethoxycinnamic acid (182.3 g, 865 mmol, 1.0 equiv) and dichloroethane (2.5 L). Add boron tribromide (163.5 mL, 433.2 g, 1.73 mol, 2.0 equiv.) dropwise over 1 h, keeping the temperature below 35° C. Gas evolution can be monitored as the temperature of the reaction is gradually increased to reflux (82° C.). Reflux for 12 h, cool to 5° C., and quench by the careful addition of water (1.0 L). Filter the resulting yellow-red suspension/emulsion through a glass frit and wash with dichloroethane (1.0 L) and heptane (1.0 L) to afford a brown solid. Dry the wet material in a vacuum oven (30 in., 35° C.) for 18 h, to afford the coumarin (180.3 g, 127% theory) as a brown solid: ¹H NMR (300 MHz, DMSO-d₆) δ 7.97 (d, J=9.6 Hz, 1H), 7.22 (d, J=9.9 Hz, 1H), 7.05 (m, 2H), 6.43 (d, J=9.6 Hz, 1H).

Equip a 5-L, three-neck, round-bottom flask with a mechanical stirrer, thermocouple, an addition funnel, and an inlet adapter. Charge the flask with the coumarin prepared above (360.0 g, 2.20 mol, 1.0 equiv) and N,N-dimethylformamide (2.2 L). While keeping the temperature below 30° C., add cesium carbonate [904.2 g, 2.78 mol, 1.25 equiv]. Then add benzyl bromide [475.5 g, 330.2 mL, 2.78 mol, 1.25 equiv] over a period of 1 h, keeping the temperature below 35° C. during the addition. Stir the mixture at ambient temperature (25-30° C.) for 10.5 h. Pour the reaction mixture into ice water (4.5 L), filter, and dry at ambient pressure for 72 h, triturate in heptane (1.5 L) with vigorous stirring, filter, and dry under reduced pressure (30 in., 35° C.) to afford preparation 46 (302.4 g, 1.20 mol, 60%) as a light brown solid: ¹H NMR (300 MHz, DMSO-d₆) δ 7.99 (d, J=9.6 Hz, 1H), 7.50-7.29 (m, 8H), 6.49 (d, J=9.5 Hz, 1H), 5.15 (s, 2H); ¹³C NMR (75 MHz, DMSO-d₆) δ 160.0, 154.6, 147.9, 143.9, 136.6, 128.4, 127.9, 127.7, 119.9, 119.1, 117.3, 116.6, 111.9, 69.8; IR (KBr) 3052 (w), 1708 (s), 1568 (m), 1492 (w), 1444 (w), 1383 (w), 1272 (m), 1168 (w), 1110 (m), 1020 (m), 927 (w), 814 (w), 762 (w), 709 (w) cm⁻¹; HPLC analysis 95.9% (AUC), Phenomenex Luna C18(2) column; ESI MS m/z 253 [C₁₆H₁₂O₃+H]⁺

### Preparation 47

### 8-Benzyloxy-2-methylene-2,3,3a,9b-tetrahydro-1H-cyclopenta[c]chromen-4-one

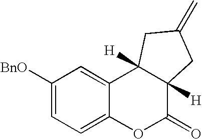

Starting from preparation 46 this compound can be prepared in a manner substantially similar to that described in preparation 13. ¹H NMR (400 MHz, CDCl₃) δ 7.44-7.32 (m, 5H), 6.98 (d, J=8.8 Hz, 1H), 6.86 (dd, J=3.1, 8.8 Hz, 1H), 6.82 (d, J=3.1 Hz, 1H), 5.04 (s, 2H), 4.98-4.95 (m, 2H), 3.40 (dt, J=7.5, 16.3 Hz, 1H), 3.15 (ddd, J=4.4, 7.9, 11.9 1H, 3.06-3.01 (m, 1H), 2.82-2.72 (m, 2H), 2.47-2.40 (m, 1H).

### Preparation 48

### 8-Benzyloxy-4-(4-benzyloxy-phenyl)-2-methylene-1,2,3,9b-tetrahydro-cyclopenta[c]chromene

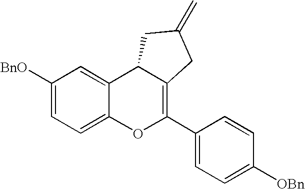

Add p-benzyloxybromobenzene (20 g, 76 mmol) to magnesium metal (1.85 g, 76 mmol). Flush with nitrogen and add 76 mL of THF followed by a small crystal of I₂. Heat to reflux to initiate Grignard formation and then let stir at room temperature overnight. Add the resulting aryl Grignard via cannula to a solution of ZnCl₂ (76 mL of a 1 M solution in Et₂O, 76 mmol) in 152 mL of THF. Stir for 30 min and then let the precipitate settle to give a solution of the aryl zinc.

Cool a solution of preparation 47 (9.43 g, 30.8 mmol) in 312 mL of THF to −78° C. Add KHMDS (74 mL of a 0.5 M solution in toluene, 37 mmol). Stir for 20 min. Add via cannula a solution of N-phenyl bis(trifluoromethanesulphonamide) (13.22 g, 37 mmol) in 47 mL of THF. Stir for 2 hrs and then quench with saturated aqueous NH₄Cl. Partition the solution between 250 mL of 1:1 water:brine and 250 mL of EtOAc. Separate and wash the organic solution with brine, dry over Na₂SO₄, filter, and concentrate. Adsorb the material to silica gel and purify by silica gel chromatography eluting with a linear gradient of 0-100% CH₂Cl₂ in hexanes to afford 9.77 g (22.3 mmol, 72%) of the enol triflate of preparation 46.

Add via cannula the solution of the aryl zinc described above to a solution of the enol triflate described above and Pd(PPh₃) (2.57 g, 2.22 mmol) in 36 mL of THF under N₂. Heat the solution to 50° C. for 30 min. Cool the solution to room temperature and quench with saturated aqueous sodium bicarbonate and extract with EtOAc. Wash the combined organic solutions with brine, dry over Na₂SO₄, filter and concentrate. To remove the catalyst, dissolve the residue in 1:1 hexanes:CH₂Cl₂ and filter through celite. Further purify the product by filtration through silica gel using 1:1 hexanes:CH₂Cl₂. Further purify by re-crystallization from EtOAc and hexanes to afford 5.96 g (12.6 mmol, 57%) of preparation 48. ¹H NMR (400 MHz, CDCl₃) δ 7.58-7.38 (m, 12H), 7.06-7.03 (m, 3H), 6.87 (dd, J=2.6, 8.8 Hz, 1H), 6.79 (d, J=3.1 Hz, 1H), 5.15 (s, 2H), 5.09 (s, 2H), 5.06 (s, 1H), 5.00 (s, 1H), 3.95 (t, J=9.7 Hz, 1H), 3.48 (d, J=20 Hz, 1H), 3.33 (d, J=20 Hz, 1H), 3.12 (dd, J=8.4, 15.4 Hz 1H), 2.50 (t, J=12.8 Hz, 1H).

### Preparation 49

### 8-Benzyloxy-4-(4-benzyloxy-phenyl)-2-methylene-1,2,3,3a,4,9b-hexahydro-cyclopenta[c]chromene

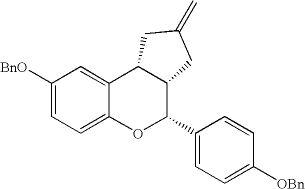

Add TFA (3.2 mL, 41.6 mmol) to a solution of preparation 48 (5.94 g, 12.6 mmol) and Et₃SiH (20.1 mL, 126 mmol) in 101 mL of CH₂Cl₂ at 0° C. Stir for 5 min and then pour into a solution of saturated aqueous sodium bicarbonate. Wash the organic solution two times with saturated aqueous sodium bicarbonate, dry over Na₂SO₄, filter, and concentrate. Purify the product by silica gel chromatography eluting with 10-60% CH₂Cl₂ in hexanes to afford 3.67 g (7.73 mmol, 62%) of preparation 49. ¹H NMR (400 MHz, CDCl₃) δ 7.50-7.35 (m, 12H), 7.29-7.02 (m, 2H), 6.88 (d, J=9.2 Hz, 1H), 6.83-6.79 (m, 2 H), 5.14 (d, J=1.8 Hz, 1H), 5.12 (s, 2H), 5.05 (s, 2H), 4.78 (m, 2H), 3.60 (t, J=7.5 Hz, 1H), 2.92 (m, 1H), 2.73 (m, 1H), 2.65 (d, J=16.7 Hz, 1H), 2.46 (m, 1H), 2.13 (dd, J=7.9, 16.7 Hz, 1H).

### Preparation 50

### 8-Benzyloxy-4-(4-benzyloxy-phenyl)-1,3a,4,9b-tetrahydro-3H-cyclopenta[c]chromen-2-one

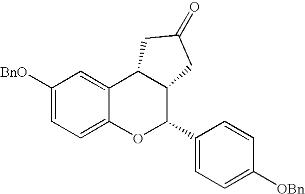

Add osmium tetroxide (4.8 mL of a 2.5 wt % solution in t-BuOH, 0.38 mmol) to a solution of preparation 49 (3.62 g, 7.63 mmol), N-methylmorpholine (0.84 mL, 7.6 mmol), and N-methylmorpholine-N-oxide (1.79 g, 15.3 mmol) in 55 mL of THF and 21 mL of water. Stir for 6.5 hrs and then add 88 mL of THF, 106 mL of water and sodium periodate (8.16 g, 38.2 mmol). Stir overnight. Quench with an 1:1 solution of saturated aqueous Na₂SO₃ and saturated aqueous NaHCO₃. Separate the organic solution and wash with brine, dry over Na₂SO₄, filter and concentrate. Dissolve in 1:1 EtOAc:CH₂Cl₂ and wash with water, dry over Na₂SO₄, filter and concentrate to afford 3.35 g (7.03 mmol, 92%) of preparation 50. ¹H NMR (400 MHz, CDCl₃) δ 7.49-7.34 (m, 12H), 7.04-7.02 (m, 2H), 6.92 (d, J=8.8 Hz, 1H), 6.85 (dd, J=2.6, 8.8Hz, 1H), 6.77 (d, J=2.6 H), 5.16 (s, 1H), 5.12 (s, 2H), 5.04 (s, 2H), 3.90 (t, J=7.5 Hz, 1H), 2.96 (dt, J=3.0, 13.7 Hz, 1H), 2.80 (dd, J=8.4, 18.5 Hz, 1H), 2.63 (d, J=18.1 Hz, 1H), 2.37 (dd, J=11.9, 18.9 Hz, 1H), 2.08 (dd, J=7.9, 18.5 Hz, 1H).

### Preparation 51

### 8-benzyloxy-4-(4-benzyloxy-phenyl)-1,2,3,3a,4,9b-hexahydro-cyclopenta[c]chromen-2-ol

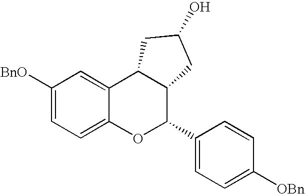

Add sodium borohydride (240 mg, 6.3 mmol) to a solution of preparation 50 (1.5 g, 3.15 mmol) in 30 mL of THF and 30 mL of methanol. Let stir for 30 min. Quench with saturated aqueous ammonium chloride, separate, back extract the aqueous solution two times with EtOAc. Combine the organic solutions and wash with 1:1 brine:water, dry over Na₂SO₄, filter, and concentrate to give 1.5 g (3.13 mmo, 99%) of preparation 51. ¹H NMR (400 MHz, CDCl₃): δ 7.56-7.33 (m, 12H), 7.05-7.01 (m, 2H), 6.92 (d, J=8.8 Hz, 1H), 6.83-6.80 (m, 2H), 5.12 (s, 2H), 5.07 (s, 1H), 5.06 (s, 2H), 4.27 (d, J=6.6, 11.0 Hz, 1H), 3.53 (m, 1H), 2.63 (m, 1H), 2.51 (dt, J=7.5, 13.6 Hz, 1H),1.92-1.86 (m, 2H), 1.72 (dddd, J=6.6, 11.0, 13.6, 17.1 Hz, 1H). HRMS (ES+) calc: 496.2488; found: 496.2485 [M+NH₄]⁺.

### Preparation 52

### 8-Benzyloxy-4-(4-benzyloxy-phenyl)-1,2,3,3a,4,9b-hexahydro-cyclopenta[c]chromene-2-carbonitrile

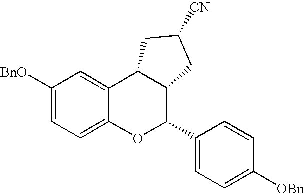

To a solution of preparation 51 (50 mg, 0.104 mmol), acetone cyanohydrin (48 μL, 0.52 mmol), and triphenyl phosphine (137 mg, 0.52 mmol), in 2.5 mL of THF at 0° C. add diisopropylazodicarboxylate (103 μL, 0.52 mmol). Stir the solution and allow it to warm slowly to room temperature overnight. Add 1 g of silica gel and concentrate. Purify by silica gel chromatography eluting with 10-30% EtOAc in hexanes to afford 30 mg (0.62 mmol, 59%) of preparation 52. ¹H NMR (400 MHz, CDCl₃) δ 7.63-7.33 (m, 12H), 7.06-7.02 (m, 2H), 6.90 (d, J=8.8 Hz, 1H), 6.83 (dd, J=3.1, 8.8 Hz, 1H), 6.77 (d, J=3.1 Hz, 1H), 5.13 (s, 2H), 5.07 (d, J=2.2 Hz, 1H), 5.06 (s, 2H), 3.70 (t, J=6.6 Hz, 1H), 2.97 (ddt, J=2.2, 9.2, 18.9 Hz, 1H), 2.68 (m, 1H), 2.43 (m, 1H), 2.28 (ddd, J=1.8, 7.0, 8.8 Hz, 1H), 2.15 (dt, J=9.2, 13.6 Hz, 1H), 1.80 (ddd, J=6.2, 9.3, 13.6 Hz, 1H); HRMS(FAB) calcd. for C₃₃H₂₉NO₃: 487.2147; found: 487.2124 (M+).

### EXAMPLE 24

### (2R, 3aR, 4S, 9bS)- and (2S, 3aS, 4R, 9bR)-8-Hydroxy-4-(4-hydroxy-phenyl)-1,2,3,3a,4,9b-hexahydro-cyclopenta[c]chromene-2-carbonitrile

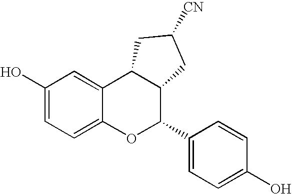

Dissolve preparation 52 (24 mg, 0.050 mmol) in 1 mL of THF. Add a slurry of 10% Pd/C (10 mg) in 1 mL of iPrOH. Add another 1 mL of THF, warm to redissolve preparation 52, then stir under an atmosphere of hydrogen gas at ambient pressure for 6 hrs. Filter the solution through a 0.2 μm HPLC filter, wash with methanol and concentrate. Purify by silica gel chromatography eluting with 5-50% (9:1 EtOAc:MeOH) in hexanes to afford 11.2 mg (0.036, 73%) of example 26. ¹H NMR (400 MHz, CDCl₃): δ 7.32-7.30 (m, 2H), 6.85-6.82 (m, 2H), 6.76 (d, J=8.4 Hz, 1H), 6.65 (d, J=2.6 Hz, 1H), 6.61 (dd, J=3.0, 8.7 Hz, 1H), 5.00 (d, J=2.5 Hz, 1H), 3.67 (t, J=6.6 Hz, 1H), 2.99 (dt, J=2.2, 9.7 Hz, 1H), 2.72 (ddd, J=7.0, 9.7, 13.6 Hz, 1H), 2.41 (ddd, J=7.0, 9.7, 12.7 Hz, 1H), 2.29 (ddd, J=1.6, 6.6, 8.8 Hz, 1H), 2.11 (ddd, J=9.2, 13.6, 18.0 Hz, 1H), 1.68 (ddd, J=6.6, 9.2, 13.2 Hz, 1H). HPLC (Zorbax C18 column; 10 to 100% CH₃CN/H₂O for 10 min then 100% CH₃CN for 5 min; 1 mL/min; tᵣ 9.064 min). HRMS(ES−) calcd. for C₁₉H₁₆NO₃: 306.1130; found: 306.1155 (M−1).

### Preparation 53

### 8-Benzyloxy-4-(4-benzyloxy-phenyl)-1,2,3,3a,4,9b-hexahydro-cyclopenta[c]chromene-2-carbonitrile

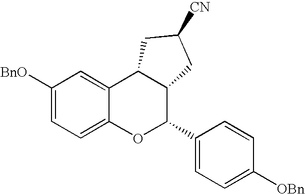

Place preparation 51 (0.2619 g, 0.5472 mmol) and triphenylphosphine (0.29 g, 1.1 mmol) in a flask and flush with N₂. Add THF (5.5 mL) and p-nitrobenzoic acid (0.27 g 1.6 mmol) and cool to 0° C. Add diisopropylazodicarboxylate (0.22 mL, 1.1 mmol) dropwise to the reaction mixture keeping it below 5° C. Let the reaction mixture warm slowly to room temperature overnight. Dilute the solution with EtOAc (100 mL), wash with saturated aqueous sodium bicarbonate (2×50 mL), brine (50 mL), dry over Na₂SO₄, filter and concentrate. Purify by silica gel chromatography (10-25% of 9:1 CH₂Cl₂:EtOAc in hexanes over 30 min) to afford 0.1876 g (0.2989 mmol, 55%) of a yellow solid. ¹H NMR (400 MHz, CDCl₃): δ 8.30 (d, 2H, J=8.8 Hz), 8.71 (d, 2H, J=8.8 Hz), 7.35-7.49 (m, 10 H), 7.41 (d, 2H, J=8.8 Hz), 7.03 (d, 2H, J=8.4 Hz), 6.91 (d, 1H, J=8.8 Hz), 6.81-6.85 (m, 2H), 5.41 (m, 1H), 5.14 (m, 1H), 5.11 (s, 2H), 5.06 (s, 2H), 3.74-3.77 (m, 1H), 2.95-3.02 (m, 1H), 2.49-2.55 (m, 1H), 2.36-2.42 (m, 1H), 2.16-2.24 (m, 1H), 1.73 (dd, 1H, J=7.5 Hz, J=14 Hz). HRMS (CI+) calcd. for C₃₉H₃₃NO₇: 627.2257; found: 627.2263 (M+).

Dissolve the yellow solid (0.1839 g, 0.2930 mmol) in 2.9 mL of THF and add an aqueous solution of LiOH (0.035 g, 1.5 mmol) in 1.1 mL of water. Stir at room temperature overnight. Add 1.0 M aqueous NaH₂PO₄ (1.5 mL, 1.5 mmol). Dilute with EtOAc (100 mL), wash with saturated aqueous NaHCO₃ (2×50 mL), wash with brine (50 mL), dry over Na₂SO₄, filter and concentrate to afford a white solid (0.1398 g, 0.2921 mmol, 99%). ¹H NMR (400 MHz, CDCl₃): δ 7.36-7.49 (m, 12H), 7.03 (d, 2H, J=8.8 Hz), 6.87-6.89 (m, 1H), 6.78-6.80 (m, 2H), 5.12 (s, 2H), 5.10-5.11 (m, 1H), 5.05 (s, 2H), 4.34 (m, 1H), 3.65-3.70 (m, 1H), 2.98-3.06 (m, 1H), 2.26-2.32 (m, 1H), 2.04-2.10 (m, 1H), 1.87-1.95 (m, 1H), 1.44 (dd, 1H, J=7.9 Hz, J=14 Hz), 1.30 (m, 1H). HRMS (CI+) calcd. for C₃₂H₃₀O₄: 478.2144; found: 478.2154 (M+).

Dissove the white solid (0.1092 g, 0.2282 mmol) and PPh₃ (0.30 g, 1.1 mmol) in THF (5.5 mL). Add acetone cyanohydrin (0.42 mL, 4.6 mmol) and cool to 0° C. Add diisopropyl azodicarboxylate (0.22 mL, 1.1 mmol) dropwise keeping the solution below 5° C. Let the solution warm slowly to room temperature overnight. Add silica gel and concentrate. Purify by silica gel chromatography eluting with CH₂Cl₂ in EtOAc to afford 0.0432 g (0.0886 mmol, 39%) of preparation 53. ¹H NMR (400 MHz, CDCl₃): δ 7.35-7.50 (m, 12H), 7.04 (d, 2H, J=8.8 Hz), 6.92 (d, 1H, J=8.8 Hz), 6.84 (dd, 1H, J=8.8 Hz, J=3.1 Hz), 6.76 (d, 1H, J=3.1 Hz), 5.13 (s, 2H), 5.06 (s, 2H), 5.05 (m, 1H), 3.59-3.65 (m, 1H), 2.70-2.80 (m, 2H), 2.59-2.67 (m, 1H), 2.00-2.10 (m, 2H), 1.84-1.91 (m, 1H). HRMS calcd. for C₃₃H₂₉NO₃: 487.2147; found: 487.2134 (M+).

### EXAMPLE 25

### (2S, 3aR, 4S, 9bS)- and (2R, 3aS, 4R, 9bR)-8-Hydroxy-4-(4-hydroxy-phenyl)-1,2,3,3a,4,9b-hexahydro-cyclopenta[c]chromene-2-carbonitrile

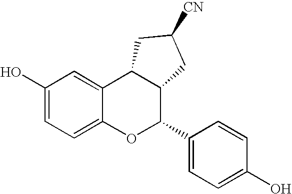

Example 27 can be prepared in a manner substantially similar to Example 26 except starting from Preparation 53. The hydrogenation is carried out under a 60 psi atmosphere of hydrogen for several days. ¹H NMR (δ, 400 MHz, CDCl₃): 7.30 (d, 2H, J=8.4 Hz), 6.83 (d, 2H, J=8.8 Hz), 6.78 (d, 2H, J=8.8 Hz), 6.64-6.56 (m, 1H), 6.62 (dd, 1H, J=8.4, 2.6 Hz), 4.99 (m, 1H), 3.55-3.62 (m, 1H), 2.90-3.00.(m, 1H), 2.67-2.76 (m, 2H), 1.94-2.04 (m, 2H), 1.78-1.94 (m, 2H). HPLC (Zorbax C18 column; 10 to 100% CH₃CN/H₂O for 10 min then 100% CH₃CN for 5 min; 1 mL/min; tᵣ 8.873 min). HRMS(CI+) calcd. for C₁₉H₁₇NO₃: 307.1208; found: 307.1212 [M+].

### Preparation 54

### 8-(tert-Butyl-dimethyl-silanyloxy)-4-[4-(tert-butyl-dimethyl-silanyloxy)-phenyl]-1,3a,4,9b-tetrahydro-3H-cyclopenta[c]chromen-2-one

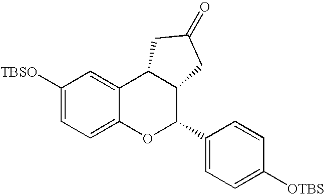

Dissolve Preparation 50 (1.59 g, 3.33 mmol) in 50 mL of THF. Add a slurry of 10% Pd/C (570 mg) in isopropyl alcohol. Stir the solution under 60 psi of hydrogen gas overnight. Filter the solution through celite and wash with isopropyl alcohol and THF. Combine and concentrate the organic solutions to afford a tan solid. Dissolve the solid 17 mL of DMF. Add imidazole (1.36 g, 20 mmol) and DMAP (42 mg, 0.34 mmol) followed by TBSCl (1.10 g, 7.3 mmol). Let the solution stir overnight. Dilute with EtOAc and wash with saturated aqueous sodium bicarbonate, water, brine, dry over Na₂SO₄, filter, and concentrate. Purify by silica gel chromatography eluting with 0-10% EtOAc in hexanes to afford 1.15 g (2.19, 66%) of preparation 54. ¹H NMR (400 MHz, MeOD): δ 7.33-7.30 (m, 2H), 6.90-6.87 (m, 2H), 6.85 (d, J=8.8 Hz, 1H), 6.68 (dd, J=2.2, 8.8 Hz, 1H), 6.63 (d, J=2.2 Hz, 1H), 5.15 (d, J=1.8 Hz, 1H), 3.87 (t, J=7.5 Hz, 1H), 2.93 (m, 1H), 2.81 (dd, J=7.6, 17.6 Hz, 1H), 2.62 (d, J=18.5 Hz, 1H), 2.36 (dd, J=12.3, 18.8 Hz, 1H), 2.04 (dd, J=7.9, 18.8 Hz, 1H), 1.02 (s, 9H), 1.01 (s, 9H), 0.24 (s, 6H), 0.21 (s, 6H).

### EXAMPLE 26

### (3aR, 4S, 9bS)- and (3aS, 4R, 9bR)-4-(4-Hydroxy-phenyl)-2-methylene-1,2,3,3a,4,9b-hexahydro-cyclopenta[c]chromen-8-ol

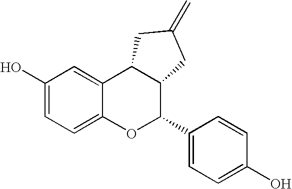

Add KHMDS (5.7 mL of 0.5 M solution in toluene, 2.85 mmol) to a solution of methyltriphenylphosphonium bromide (1.2 g, 3.36 mmol) in 30 mL of THF at −78° C. Stir for 30 min and then add via cannula a solution of preparation 54 (500 mg, 0.95 mmol) in 10 mL of THF followed by 2×5 mL THF washes. Remove cooling bath and let stir overnight. Quench with saturated aqueous ammonium chloride. Dilute with EtOAc wash with 1:1 brine:water, brine, dry over Na₂SO₄, filter and concentrate. Adsorb to silica gel and purify by silica gel chromatography eluting with 0-100% EtOAc in hexanes to afford the title compound in addition to mono and di-TBS protected material. Repeat the procedure starting with 250 mg of preparation 54 except stir for only 3 hrs. Combine the di-TBS protected material (219 mg, 0.42 mmol) and dissolve in 5 mL of THF. Add TBAF (0.88 mL of a 1 M solution in THF, 0.88 mmol). Let stir for 15 min and quench with saturated aqueous sodium carbonate. Dilute with water and EtOAc. Separate and extract the aqueous solution with EtOAc. Combine the organic solutions, add a little methanol, and wash with brine, dry over Na₂SO₄, filter and concentrate. Repeat the same procedure with the combined mono-TBS protected material. Combine all the deprotected material and adsorb to 5 g of silica gel. Purify by silica gel flash chromatography eluting with 10-40% (9:1 EtOAc:MeOH) in hexanes to afford 255 mg (0.87 mmol, 75%) of example 28. The two enantiomers can be separated by chiral preparative HPLC (Chiralpak AD, MeOH). ¹H NMR (400 MHz, MeOD): δ 7.32-7.29 (m, 2H), 6.84-6.80 (m, 2H), 6.72 (d, J=8.8 Hz, 1H), 6.64 (d, J=3.1 Hz, 1H), 6.58 (d, J=3.1, 8.8 Hz, 1H), 5.05 (d, J=1.8 Hz, 1H), 4.74 (d, J=13.2 Hz, 2H), 3.54 (t, J=7.5 Hz, 1H), 2.95-2.88 (m, 1H), 2.77-2.69 (m, 1H), 2.61 (d, J=16.3 Hz, 1H), 2.40-2.32 (m, 1H), 2.05 (dd, J=8.8, 16.7 Hz, 1H); HPLC (Zorbax C18 column; 10 to 100% CH₃CN/H₂O for 10 min then 100% CH₃CN for 5 min; 1 mL/min; tᵣ 9.838 min; HRMS(ES−) calcd. for C₁₉H₁₇O₃: 293.1178; found: 293.1148 [M−1]. HPLC (Chiralpak AD, 15% EtOH/Heptane, 1 mL/min; t_{R}=9.0 min (enatiomer A); 13.4 min (enantiomer B).

### Preparation 55

### 8-(tert-Butyl-dimethyl-silanyloxy)-4-[4-(tert-butyl-dimethyl-silanyloxy)-phenyl]-2-difluoromethylene-1,2,3,3a,4,9b-hexahydro-cyclopenta[c]chromene

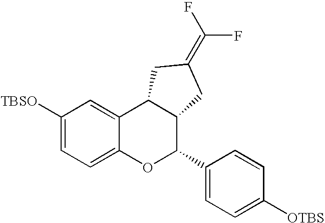

Dissolve diisopropylamine (67 μL, 0.48 mmol) in 2 mL of THF, cool to −50° C. and add n-butyllithium (238 μL of a 1.6 M solution in hexanes, 0.38 mmol). Then add a solution of (Difluoromethyl)diphenylphosphine oxide (prepared according to Edwards, M. L.; Stemerick, D. M.; Jarvi, E. T.; Matthews, D. P.; McCarthy, J. R. Tetrahedron Lett. 1990, 31, 5571-5574) in 0.5 mL of THF via cannula followed by a 0.5 mL wash. Let stir for 30 min and then add preparation 54 (100 mg, 0.19 mmol) as a solution in 0.5 mL of THF via syringe followed by a 0.5 mL wash. Let stir and allow to warm slowly to 0° C. over 2 hrs. Remove the cooling bath and let warm to room temperature and then warm to reflux for 1 hr. Cool the solution to room temperature and quench with saturated aqueous ammonium chloride. Dilute the solution with EtOAc, wash with brine, dry over Na₂SO₄, filter and concentrate. Adsorb to 1 g of silica gel and purify by silica gel chromatography eluting with 5-20% EtOAc to afford 41 mg (0.073 mmol, 39%) preparation 55. ¹H NMR (400 MHz, MeOD): δ 7.35-7.30 (m, 2H), 6.91-6.88 (m, 2H), 6.83 (d, J=8.3 Hz, 1H), 6.69-6.65 (m, 2H), 5.14 (s, 1H), 3:60 (m, 1H), 2.82 (m, 1H), 2.74-2.64 (m, 2H), 2.42 (m, 1H), 2.09 (dd, J=8.3, 15.8 Hz, 1H), 1.03 (s, 9H), 1.02 (s, 9H), 0.24 (s, 6H), 0.22 (s, 6H).

### EXAMPLE 27

### (3aR, 4S, 9bS)- and (3aS, 4R, 9bR)-2-Difluoromethylene-4-(4-hydroxy-phenyl)-1,2,3,3a,4,9b-hexahydro-cyclopenta[c]chromen-8-ol

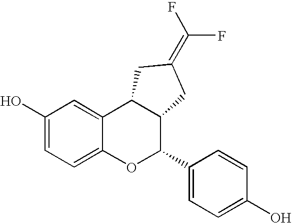

Add TBAF (135 μL of a 1M solution in THF, 0.135 mmol) to a solution of preparation 55 (38 mg, 0.068 mmol) in 1 mL of THF. Let stir for 5 min, add one more drop of TBAF and then quench with aqueous sodium bicarbonate. Dilute with EtOAc, wash with water, brine, dry over Na₂SO₄, filter and concentrate. Adsorb to 0.5 g of silica gel and purify by silica gel chromatography eluting with 5-50% (9:1 EtOAc:MeOH) in hexanes to afford 20 mg (0.061 mmol, 89%) of example 29. ¹H NMR (400 MHz, MeOD): δ 7.31 (m, 2H), 6.83 (m, 2H), 6.74 (d, J=8.8 Hz, 1H), 6.67 (d, J=2.6 Hz, 1H), 6.61 (dd, J=2.6, 8.8 Hz, 1H), 5.07 (s, 1H), 3.60 (m, 1H), 2.86-2.73 (m, 2H), 2.64 (d, J=15.4 Hz, 1H), 2.34 (m, 1H), 2.04 (m, 1H); HPLC (Zorbax C18 column; 10 to 100% CH₃CN/H₂O for 10 min then 100% CH₃CN for 5 min; 1 mL/min; tᵣ 10.094 min; HRMS(ES−) calcd. for C₁₉H₁₅F₂O₃: 329.0989; found: 329.0999 [M−1].

### Preparation 56

### 8-(tert-Butyl-dimethyl-silanyloxy)-4-[4-(tert-butyl-dimethyl-silanyloxy)-phenyl]-2-trimethylsilanylethynyl-1,2,3,3a,4,9b-hexahydro-cyclopenta[c]chromene

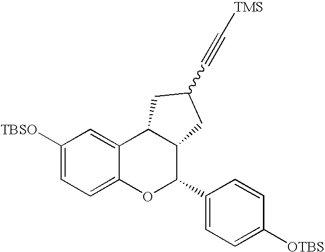

Add 1 mL of dry THF to dry cerium trichloride (120 mg, 0.22 mmol, prepared from cerium trichloride heptahydrate according to cerium(III) chloride in the Encyclopedia of Reagents for Organic Synthesis, Wiley Interscience), stir for 1 hr, and then cool to 0° C. In a separate flask add n-butyllithium (0.286 mL of a 1.6 M solution in hexanes, 0.46 mmol) to a solution of trimethylsilylacetylene (80 □L, 0.57 mmol) in 1 mL of THF cooled to −78° C. Add this solution to the cerium trichloride via cannula. Then add via cannula a solution of preparation 54 (120 mg, 0.22 mmol) in 1 mL of THF followed by 2×0.5 mL THF washes. Let stir for 3 hrs. Prepare another solution of lithiated trimethylsilylacetylene as described above and add it to the reaction flask via cannula. Let stir for 1 hr. Quench the reaction with saturated aqueous ammonium chloride, dilute with EtOAc, separate and extract the aqueous solution with EtOAc. Combine the organic solutions and wash with water, brine, dry over Na₂SO₄, filter and concentrate.

Dissolve the material in 2 mL of CH₂Cl₂. Add DMAP (3 mg, 0.024 mmol), triethylamine (0.096 mL, 0.69 mmol), and then add methylchlorooxoacetate (0.032 mL, 0.34 mmol). Let stir for 1 hr. Quench with saturated sodium bicarbonate and separate. Wash the organic solution with 1 M aqueous NaH₂PO₄, saturated aqueous sodium bicarbonate, brine, dry over Na₂SO₄, filter and concentrate. Adsorb to 1 g of silica gel and purify by silica gel flash chromatography eluting with 0-15% EtOAc in hexanes.

Dissolve the material in 1.5 mL of toluene. Add triphenyltin hydride (163 mg, 0.464 mmol) and AIBN (4 mg, 0.024 mmol). Warm the solution to 80° C. Let stir for 1 hr. Remove heat and let sit for 3 hrs. Filter through a glass frit and wash precipitate with ether. Combine the filtrates and concentrate. Adsorb to 1.2 g of silica gel and purify by silica gel chromatography eluting with 0-50% CH₂Cl₂ in hexanes to afford 56 mg (0.092 mmol) of preparation 56 as a 5:1 diastereomeric mixture of products. ¹H NMR (400 MHz, CDCl₃) of major diastereomer: δ 7.37-7.27 (m, 2H), 6.89-6.85 (m, 2H), 6.81 (d, J=8.4 Hz, 1H), 6.65-6.60 (m, 2H), 4.98 (d, J=2.2 Hz, 1H), 3.49 (m, 1H), 2.75-2.50 (m, 3H), 1.81-1.66 (m, 3H), 1.03 (s, 9H), 1.02 (s, 9H), 0.24 (s, 6H), 0.22 (s, 6H), 0.10 (s, 9H).

### EXAMPLE 28

### (3aR, 4S, 9bS)- and (3aS, 4R, 9bR)-2-Ethynyl-4-(4-hydroxy-phenyl)-1,2,3,3a,4,9b-hexahydro-cyclopenta[c]chromen-8-ol

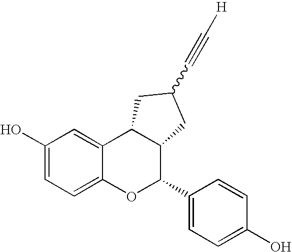

Add TBAF (0.28 mL of a 1 M solution in THF, 0.28 mmol) to a solution of preparation 56 (56 mg, 0.092 mmol) in 2 mL of THF. Let stir for 10 min. Quench with saturate sodium bicarbonate and dilute with EtOAc. Separate and wash the organic solution with water, brine, dry over Na₂SO₄, filter and concentrate. Adsorb to 0.5 g silica gel. Purify by silica gel chromatography eluting with 0-50% (9:1 EtOAc:MeOH) in hexanes to afford 11.3 mg (0.037 mmol) of example 30 as a 5:1 mixture of diastereomers. ¹H NMR (400 MHz, MeOD) of major diastereomer: δ 7.27 (m, 2H), 6.83-6.81 (m, 2H),6.74 (d, J=8.0 Hz, 1H), 6.64-6.56 (m, 2H), 4.89 (d, J=3.1 Hz, 1H), 3.48 (dt, J=8.8, 5.7 Hz, 1H), 2.74-2.57 (m, 3H), 2.22 (d, J=2.6 Hz), 1.64-1.57 (m, 3H); HPLC (Zorbax C18 column; 10 to 100% CH₃CN/H₂O for 10 min then 100% CH₃CN for 5 min; 1 mL/min; tᵣ 9.683 min (major), 9.805 (minor); HRMS(ES−) calcd. for C₂₀H₁₇O₃: 305.1178; found: 305.1170 [M−1].

### Preparation 57

### 8-Benzyloxy-4-(4-benzyloxy-phenyl)-2-ethylidene-1,2,3,3a,4,9b-hexahydro-cyclopenta[c]chromene

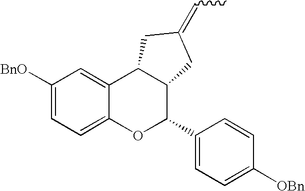

Heat a solution of preparation 50, (0.1562 g, 0.3278 mmol) in dry THF (6 mL) to dissolve. Cool a solution of ethyltriphenylphosphonium bromide (0.49 g, 1.3 mmol) in dry THF (10 mL) to −78° C. and then add KHMDS (2.2 mL of a 0.5 M solution in toluene, 1.1 mmol). After stirring at −78° C. for 15 min, add the solution of preparation 50 dropwise via cannula. Allow the solution to warm to RT for 2 h, then quench with saturated aqueous ammonium chloride (25 mL) and water (25 mL) and extract with EtOAc (3×50 mL). Wash the combined organic solutions with brine (50 mL), dry over Na₂SO₄, filter and concentrate. Purify by silica gel flash chromatography eluting with 0-50% CH₂Cl₂ in hexanes to afford 0.1552 g (0.3179 mmol, 97%) of preparation 57 as mixture of E and Z isomers. ¹H NMR (400 MHz, CDCl₃): δ 7.36-7.50 (m, 12H, 2 isomers), 7.02-7.06 (m, 2H, 2 isomers), 6.78-6.89 (m, 4H, 2 isomers), 5.22 (m, 1 H, 2 isomers), 5.17 (d, 1H, J=1.8 Hz, 1 isomer), 5.15 (d, 1H, J=1.3 Hz, 1 isomer), 5.13 (s, 2H, 1 isomer), 5.12 (s, 2H, 1 isomer), 5.06 (s, 2H, 1 isomer), 5.05 (s, 2H, 1 isomer), 3.63 (t, 1H, J=7.9 Hz, 1 isomer), 3.54 (t, 1H, J=7.0 Hz, 1 isomer), 2.60-2.85 (m, 3H, 2 isomers), 2.41-2.48 (m, 1H, 1 isomer), 2.25-2.32 (m, 1H, 1 isomer), 2.03-2.12 (m, 1H, 2 isomers), 1.52 (d, 3H, J=6.6 Hz, 1 isomer), 1.47 (d, 3H, J=7.0 Hz, 1 isomer). HRMS(CI+) calcd. for C₃₄H₃₃O₃: 489.6241; found: 489.2411 (M+1).

### Preparation 58

### 8-Benzyloxy-4-(4-benzyloxy-phenyl)-2-propylidene-1,2,3,3a,4,9b-hexahydro-cyclopenta[c]chromene

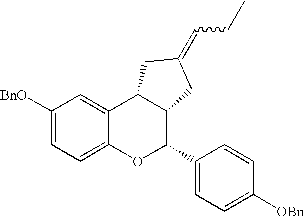

Preparation 58 can be prepared in a manner substantially similar to preparation 57 starting with preparation 50 (0.2041g, 0.4283 mmol) and propyltriphenyl-phosphonium bromide to obtain 0.1927 g (0.3834 mmol, 90%) of a mixture of E and Z isomers. ¹H NMR (400 MHz, CDCl₃): δ 7.36-7.50 (m, 12H, 2 isomers), 7.02-7.06 (m, 2H, 2 isomers), 6.79-6.89 (m, 4H, 2 isomers), 5.16 (m, 1H, 1 isomer), 5.145 (m, 1H, 1 isomer), 5.13 (s, 2H, 1 isomer), 5.12 (s, 2H, 1 isomer), 5.06 (s, 2H, 1 isomer), 5.05 (s, 2H, 1 isomer), 3.62 (t, 1H, J=7.5 Hz),3.54 (t, 1H, J=7.0 Hz, 1 isomer), 2.58-2.92 (m, 3H, 2 isomers), 2.41-2.48 (m, 1H, 1 isomer), 2.25-2.32 (m, 1H, 1 isomer), 1.85-2.11 (m, 3H, 2 isomer), 0.896 (t, 3H, J=7.5 Hz, 1-isomer), 0.863 (t, 3H, J=7.5 Hz, 1 isomer). HRMS(CI+) calcd. for C₃₅H₃₅O₃: 503.2586; found: 503.2563 (M+1) .

### Preparation 59

### 8-Benzyloxy-4-(4-benzyloxy-phenyl)-2-butylidene-1,2,3,3a,4,9b-hexahydro-cyclopenta[c]chromene

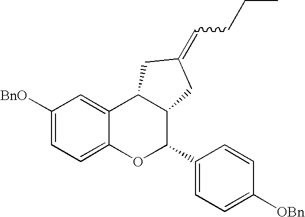

Preparation 59 can be prepared in a manner substantially similar to preparation 57 starting with preparation 50 (0.203 g, 0.427 mmol) and butyltriphenyl-phosphonium to obtain (0.1894 g, 0.3665 mmol, 86%) of a mixture of E and Z isomers. ¹H NMR (400 MHz, CDCl₃): δ 7.34-7.50 (m, 12H, 2 isomers), 7.02-7.06 (m, 2H, 2 isomers), 6.78-6.88 (m, 4H, 2 isomers), 5.14 -5.17 (m, 2H, 2 isomers), 5.13 (s, 2H, 1 isomer), 5.12 (s, 2H, 1 isomer), 5.06 (s, 2H, 1 isomer), 5.05 (s, 2H, 1 isomer), 3.61 (t, 1H, J=7.5 Hz, 1 isomer), 3.54 (t, 1H, J=6.6 Hz), 2.59-2.86 (m, 3H, 2 isomers), 2.42-2.49 (m, 1H, 1 isomer), 2.24-2.31 (m, 1H, 1 isomer), 2.03-2.11 (m, 1H, 2 isomers), 1.80-1.89 (m, 2H, 2 isomers), 1.22-1.35 (m, 2H, 2 isomers), 0.856 (t, 3H, J=7.5 Hz, 1 isomer), 0.804 (t, 3H, J=7.0 Hz, 1 isomer).

### Preparation 60

### 2-Benzylidene-8-benzyloxy-4-(4-benzyloxy-phenyl)-1,2,3,3a,4,9b-hexahydro-cyclopenta[c]chromene

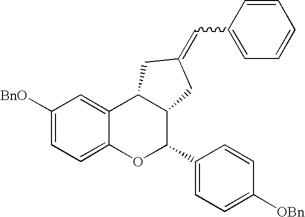

Preparation 60 can be prepared in a manner substantially similar to preparation 57 except the reaction mixture was heated to reflux overnight. Starting with preparation 50 (0.203 g, 0.427 mmol) using two addition of the Wittig reagent formed from benzyltriphenyl-phosphonium chloride affords 0.0922 g (0.167 mmol, 39%) of a mixture of E and Z isomers. ¹H NMR (400 MHz, CDCl₃): δ, 7.20-7.51(m, 17H, 2 isomers), 7.06 (d, 2H, J=8.6 Hz, 2 isomers), 6.82-6.90 (m, 4H, 2 isomers), 6.26 (s, 1H, major isomer), 5.20 (s, 1H, minor isomer), 5.18 (s, 2H, minor isomer), 5.14 (s, 2H, major isomer), 5.06 (s, 2H, minor isomer), 5.03 (s, 2H, major isomer), 3.70-3.78 (m, 1H, major isomer), 3.56-3.64 (m, 1H, minor isomer), 3.12-3.25 (m, 1H, 2 isomers), 2.24-3.00 (m, 3H, 2 isomers).

### EXAMPLE 29

### 2-Butyl-4-(4-hydroxy-phenyl)-1,2,3,3a,4,9b-hexahydro-cyclopenta[c]chromen-8-ol

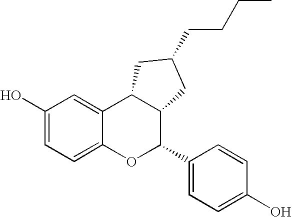

Dissolve preparation 59 (0.1829 g, 0.3540 mmol) in 11 mL of THF. Add a slurry of 10% Pd/C (0.0619 g) in 11 mL of isopropyl alcohol. Stir the solution under an atmosphere of hydrogen at ambient pressure and temperature overnight. Filter the solution through Celite and wash the filter cake with isopropyl alcohol and THF. Combine and concentrate the filtrate and washings and concentrate. Adsorb to 2 g of silica gel. Purify by silica gel flash chromatography eluting with 10-50% (1:9 MeOH/EtOAc) in hexanes to afford 0.0823 g (0.2432 mmol, 69%) of example 31. ¹H NMR (400 MHz, MeOD): δ 7.27 (d, 2H, J=8.8 Hz), 6.81 (d, 2H, J=8.4 Hz), 6.71 (d, 1H, J=8.4 Hz), 6.58 (d, 1H, J=3.1 Hz), 6.54 (dd, 1H, J=3.1 Hz, J=8.8 Hz), 3.41-3.47 (m, 1H), 2.48-2.63 (m, 2H), 1.77-1.90 (m, 1H), 1.40-1.46 (m, 1H), 1.14-1.26 (m, 8H), 0.863 (t, 3H, J=6.6 Hz). HPLC (Zorbax C18 column; 10 to 100% CH₃CN/H₂O for 10 min then 100% CH₃CN for 5 min; 1 mL/min; tᵣ 11.575 min). LRMS (ES−): 337.2 (M−1).

The two enantiomers can be separated by chiral preparative HPLC (Chiralpak AD, 15% EtOH/Heptane).

Enantiomer A: ¹H NMR (400 MHz, MeOD): δ 7.27 (d, 2H, J=8.8 Hz), 6.81 (d, 2H, J=8.4 Hz), 6.71 (d, 1H, J=8.4 Hz), 6.58 (d, 1H, J=3;1 Hz), 6.54 (dd, 1H, J=3.1 Hz, J=8.8 Hz), 3.41-3.47 (m, 1H), 2.48-2.63 (m, 2H), 1.77-1.90 (m, 1H), 1.40-1.46 (m, 1H), 1.14-1.26 (m, 8H), 0.863 (t, 3H, J=6.6 Hz). HPLC (Zorbax C18 column; 10 to 100% CH₃CN/H₂O for 10 min then 100% CH₃CN for 5 min; 1 mL/min; tᵣ 11.568 min). HPLC (Chiralpak AD, 15% EtOH/Heptane; 1 mL/min; t_{R}=3.213 min). LRMS (ES−): 337.2 (M−1).

Enantiomer B: ¹H NMR (400 MHz, MeOD): δ 7.27 (d, 2H, J=8.8 Hz), 6.81 (d, 2H, J=8.4 Hz), 6.71 (d, 1H, J=8.4 Hz), 6.58 (d, 1H, J=3.1 Hz), 6.54 (dd, 1H, J=3.1 Hz, J=8.8 Hz), 3.41-3.47 (m, 1H), 2.48-2.63 (m, 2H), 1.77-1.90 (m, 1H), 1.40-1.46 (m, 1H), 1.14-1.26 (m, 8H), 0.863 (t, 3H, J=6.6 Hz). HPLC (Zorbax C18-column; 10 to 100% CH₃CN/H₂O for 10 min then 100% CH₃CN for 5 min; 1 mL/min; tᵣ 11.578 min). HPLC (Chiralpak AD, 15% EtOH/Heptane; 1 mL/min; t_{R}=5.877 min). LRMS (ES−): 337.2 (M−1).

### EXAMPLE 30

### 4-(4-Hydroxy-phenyl)-2-propyl-1,2,3,3a,4,9b-hexahydro-cyclopenta[c]chromen-8-ol

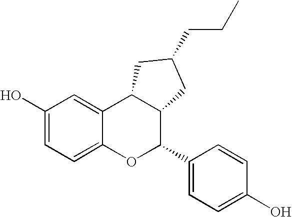

Example 32 can be prepared in a manner substantially similar to that described for example 31 starting from preparation 58 (0.1842 g, 0.3665 mmol) to afford 0.108 g (0.331 mmol, 90%). ¹H NMR (400 MHz, MeOD): δ 7.27 (d, 2H, J=8.8 Hz), 6.81 (d, 2H, J=8.8 Hz), 6.71 (d, 1H, J=8.8 Hz), 6.58 (d, 1H, J=3.1 Hz), 6.54 (dd, 1H, J=3.1 Hz, J=8.8 Hz), 3.41-3.47 (m, 1H), 2.48-2.63 (m, 2H), 1.80-1.92 (m, 1H), 1.38-1.46 (m, 1H), 1.14-1.30 (m, 6H), 0.849 (t, 3H, J=7.0 Hz). HPLC (Zorbax C18 column; 10 to 100% CH₃CN/H₂O for 10 min then 100% CH₃CN for 5 min; 1 mL/min; tᵣ 11.137 min). LRMS (ES−): 323.2 (M−1).

The two enantiomers can be separated by chiral preparative HPLC (Chiralpak AD, IPA/Heptane).

Enantiomer A: ¹H NMR (400 MHz, MeOD): δ 7.27 (d, 2H, J=8.8 Hz), 6.81 (d, 2H, J=8.8 Hz), 6.71 (d, 1H, J=8.8 Hz), 6.58 (d, 1H, J=3.1 Hz), 6.54 (dd, 1H, J=3.1 Hz, J=8.8 Hz), 3.41-3.47 (m, 1H), 2.48-2.63 (m, 2H), 1.80-1.92 (m, 1H), 1.38-1.46 (m, 1H), 1.14-1.30 (m, 6H), 0.849 (t, 3H, J=7.0 Hz). HPLC (Zorbax C18 column; 10 to 100% CH₃CN/H₂O for 10 min then 100% CH₃CN for 5 min; 1 mL/min; tᵣ 11.125 min). HPLC (Chiralpak AD, 15% EtOH/Heptane; 1 mL/min; t_{R}=3.477 min). LRMS (ES−): 323.2 (M−1).

Enantiomer B: ¹H NMR (400 MHz, MeOD): δ 7.27 (d, 2H, J=8.8 Hz), 6.81 (d, 2H, J=8.8 Hz), 6.71 (d, 1H, J=8.8 Hz), 6.58 (d, 1H, J=3.1 Hz), 6.54 (dd, 1H, J=3.1 Hz, J=8.8 Hz), 3.41-3.47 (m, 1H), 2.48-2.63 (m, 2H), 1.80-1.92 (m, 1H), 1.38-1.46 (m, 1H), 1.14-1.30 (m, 6H), 0.849 (t, 3H, J=7.0 Hz). HPLC (Zorbax C18 column; 10 to 100% CH₃CN/H₂O for 10 min then 100% CH₃CN for 5 min; 1 mL/min; tᵣ 11.127 min). HPLC (Chiralpak AD, 15% EtOH/Heptane; 1 mL/min; t_{R}=6.997 min). LRMS (ES−): 323.2 (M−1).

### EXAMPLE 31

### 2-Ethyl-4-(4-hydroxy-phenyl)-1,2,3,3a,4,9b-hexahydro-cyclopenta[c]chromen-8-ol

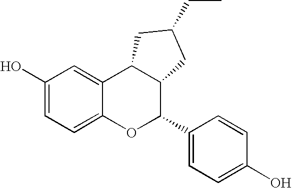

Example 33 can be prepared in a manner substantially similar to that described for example 31 starting from preparation 57. ¹H NMR (400 MHz, MeOD): δ 7.27 (d, 2H, J=8.8 Hz), 6.81 (d, 2H, J=8.4 Hz), 6.71 (d, 1H, J=8.8 Hz), 6.59 (d, 1H, J=3.1 Hz), 6.54 (dd, 1H, J=3.1 Hz, J=8.8 Hz), 3.42-3.48 (m, 1H), 2.49-2.63 (m, 2H), 1.71-1.83 (m, 1H), 1.40-1.47 (m, 1H), 1.15-1.26 (m, 4H), 0.829 (t, 3H, J=7.5 Hz). HPLC (Zorbax C18 column; 10 to 100% CH₃CN/H₂O for 10 min then 100% CH₃CN for 5 min; 1 mL/min; tᵣ 10.681 min) LRMS (ES−): 309.2 (M−1).

The two enantiomers can be separated by chiral preparative HPLC (Chiralpak AD, 15% EtOH/Heptane).

Enantiomer A: ¹H NMR (400 MHz, MeOD): □ 7.27 (d, 2H, J=8.8 Hz), 6.81 (d, 2H, J=8.4 Hz), 6.71 (d, 1H, J=8.8 Hz), 6.59 (d, 1H, J=3.1 Hz), 6.54 (dd, 1H, J=3.1 Hz, J=8.8 Hz), 3.42-3.48 (m, 1H), 2.49-2.63 (m, 2H), 1.71-1.83 (m, 1H), 1.40-1.47 (m, 1H), 1.15-1.26 (m, 4H), 0.829 (t, 3H, J=7.5 Hz). HPLC (Zorbax C18 column; 10 to 100% CH₃CN/H₂O for 10 min then 100% CH₃CN for 5 min; 1 mL/min; tᵣ 10.703 min). HPLC (Chiralpak AD, 15% EtOH/Heptane; 1 mL/min; t_{R}=3.687 min). LRMS (ES−) 309.2.

Enantiomer B: ¹H NMR (400 MHz, MeOD): □ 7.27 (d, 2H, J=8.8 Hz), 6.81 (d, 2H, J=8.4 Hz), 6.71 (d, 1H, J=8.8 Hz), 6.59 (d, 1H, J=3.1 Hz), 6.54 (dd, 1H, J=3.1 Hz, J=8.8 Hz), 3.42-3.48 (m, 1H), 2.49-2.63 (m, 2H), 1.71-1.83 (m, 1H), 1.40-1.47 (m, 1H), 1.15-1.26 (m, 4H), 0.829 (t, 3H, J=7.5 Hz). HPLC (Zorbax C18 column; 10 to 100% CH₃CN/H₂O for 10 min then 100% CH₃CN for 5 min; 1 mL/min; tᵣ 10.663 min). HPLC (Chiralpak AD, 15% EtOH/Heptane; 1 mL/min; t_{R}=8.264 min). LRMS (ES−) 309.2 (M−1).

### EXAMPLE 32

### 2-Benzyl-4-(4-hydroxy-phenyl)-1,2,3,3a,4,9b-hexahydro-cyclopenta[c]chromen-8-ol

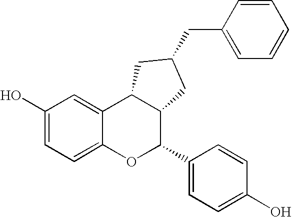

Example 34 can be prepared in a manner substantially similar to that described for example 31 starting from preparation 60 except under an atmosphere of hydrogen at 60 psi of H₂. ¹H NMR (400 MHz, MeOD): □ 7.05 (m, 7H), 6.79 (d, 2H, J=8.8 Hz), 6.74 (d, 1H, J=8.4 Hz), 6.56-6.58 (m, 2H), 4.90-4.92 (m, 1H), 3.40-3.46 (m, 1H), 2.58-2.66 (m, 1H), 2.46-2.56 (m, 1H), 2.32-2.43 (m, 2H), 2.10-2.22 (m, 1H), 1.28-1.45 (m, 3H). HPLC (Zorbax C18 column; 10 to 100% CH₃CN/H₂O for 10 min then 100% CH₃CN for 5 min; 1 mL/min; tᵣ 11.269 min). LRMS(ES−): 371.2 (M−1).

### Test Procedures

ER Binding Assay

The competition ER binding assay was run in a buffer containing 50 mM N-[2-hydroxyethyl]piperazine-N′-[2-ethanesulfonic acid (Hepes) pH 7.5, 1.5 mM EDTA, 150 mM NaCl, 10% glycerol, 1 mg/mL ovalbumin, 5mM DTT, 0.025 μCi per well of ³H-Estradiol(NEN #NET517 at 118 Ci/mmol, 1 mCi/mL), and 10 ng/well ERAlpha or ERbeta Receptor (Pan Vera). Competing compounds were added at 10 different concentrations. Non-specific binding was determined in the presence of 1 μM of E2 (17-β Estradiol, Sigma, St. Louis, Mo.). The binding reaction (140 μL) was incubated for 4 hours at room temperature, then 70 μL of cold dextran coated charcoal (DCC) buffer was added to each reaction (DCC buffer was prepared by adding 0.75 g of charcoal [Sigma] and 0.25 g of dextran [Pharmacia] per 50 mL of assay buffer). The incubation plates were mixed for 8 minutes on an orbital shaker at 4° C. and then centrifuged at 3,000 rpm for 10 minutes at 4° C. An aliquot of 120 μl of the mix was transferred to another 96-well, white flat bottom plate (Costar) and 175 μl of Wallac Optiphase Hisafe 3 scintillation fluid was added to each well. The plates were sealed and then shaken vigorously on an orbital shaker. After an incubation of 2.5 hrs, the radioactivity was counted in a Wallac Microbeta counter. The IC₅₀ and percent inhibition at 10 μM were calculated. The K_{d} for ³H-Estradiol was determined by saturation binding to ERα and ERβ receptors. The IC₅₀ values for compounds were converted to Kᵢ values using the Cheng-Prusoff equation and the K_{d} values were determined by saturation binding assay. Compounds of Examples 1-19 and 22-25 are active in the assay as described. Preferred compounds bind to the ER beta receptor with a Kᵢ of less than 20 nM. More preferred compounds bind to the ER beta receptor with a Kᵢ of less than 1 nM. Compounds that are selective to binding to the ER beta receptor compared to the ER alpha receptor bind to the ER beta receptor with a lower Kᵢ compared to the Kᵢ for the ER alpha receptor.

As determined by the above assay, the compounds of examples 1-32 exhibit binding affinities (Kis) at the ER Alpha subtype in the range 5.0→10,000 nM and to the ER beta subtype in the range of 0.20-429 nM.

LNCaP Human PCa Xenograft Assay

ERbeta agonists are evaluated for their effects on the growth of androgen-sensitive LNCaP human prostatic cancer (PCa) xenografts grown in intact sexually mature (5-6 weeks old) Hsd: Athymic Nude-nu (Athymic Nude) male mice. 2.0×10⁶ LNCaP tumor cells are injected bilaterally by the subcutaneous route into the pre-tracheal region of testicular intact male mice. Mice are castrated via the scrotal route to serve as the positive control group. Test compounds are administered once per day by subcutaneous or gavage administration at multiple dose levels in a volume of 0.2 ml to xenograft-bearing mice starting on the day following tumor injection. Test compounds are reformulated weekly based on average group mean body weights. The vehicle for these studies is 1% carboxymethyl cellulose (CMC) with 0.25% Tween 80. Body weights and tumor measurements are recorded on a weekly basis and entered directly into a JMP™ (SAS; Cary, N.C.) spreadsheet from electronic caliper measurement. Tumor volumes in mm³ are calculated in JMP using the following formula: L×W×H×0.5236. Tumor and body weight responses for individual mice are recorded on a weekly basis. When LNCaP tumor volumes enter log-phase expansion, lesions are measured every 3-4 days. Growth rates are determined using linear modeling of the log tumor values and time to treatment failure (tumor vol=1300-1500 mm³) are determined using a linear extrapolation model (SAS; Cary, N.C.). Because of humane animal use considerations, animals are sacrificed when their tumor volumes approach 1200-1400 mm³. At necropsy, final tumor measurement and body weights are recorded and whole blood is obtained via cardiac puncture and allowed to clot on ice. Serum is transferred to appropriately labeled 0.5 ml Eppendorf micro tubes, and samples are stored at −80° C. for biomarker analysis.

General Rat Preparation Procedure

Seventy-five day old (unless otherwise indicated) female Sprague Dawley rats (weight range of 200 to 225 g) are obtained from Charles River Laboratories (Portage, Mich.). The animals are either bilaterally ovariectomized (OVX) or exposed to a Sham surgical procedure at Charles River Laboratories, and then shipped after one week. Upon arrival, they are housed in metal hanging cages in groups of 3 or 4 per cage and have ad libitum access to food (calcium content approximately 0.5%) and water for one week. Room temperature is maintained at 22.2°±1.7° C. with a minimum relative humidity of 40%. The photoperiod in the room was 12 hours light and 12 hours dark.

Dosing Regimen Tissue Collection: After a one week acclimation period (therefore, two weeks post-OVX) daily dosing with a compound of formula (I) (“F-I”) is initiated. 17α-ethynyl estradiol or F-I is given orally, unless otherwise stated, as a suspension in 1% carboxymethylcellulose or dissolved in 20% cyclodextrin. Animals are dosed daily for 4 days. Following the dosing regimen, animals are weighed and anesthetized with a ketamine: Xylazine (2:1, v:v) mixture and a blood sample is collected by cardiac puncture. The animals are then sacrificed by asphyxiation with CO₂, the uterus is removed through a midline incision, and a wet uterine weight is determined. 17α-ethynyl estradiol is obtained from Sigma Chemical Co., St. Louis, Mo.

Cardiovascular Disease/Hyperlipidemia

The blood samples from above are allowed to clot at room temperature for 2 hours, and serum is obtained following centrifugation for 10 minutes at 3000 rpm. Serum cholesterol is determined-using a Boehringer Mannheim Diagnostics high performance cholesterol assay. Briefly the cholesterol is oxidized to cholest-4-en-3-one and hydrogen peroxide. The hydrogen peroxide is then reacted with phenol and 4-aminophenazone in the presence of peroxidase to produce a p-quinone imine dye, which is read spectrophotemetrically at 500 nm. Cholesterol concentration is then calculated against a standard curve. The entire assay is automated using a Biomek Automated Workstation.

Uterine Eosinophil Peroxidase (EPO) Assay

The uteri from above are kept at 4° C. until time of enzymatic analysis. The uteri are then homogenized in 50 volumes of 50 mM Tris buffer (pH 8.0) containing 0.005% Triton X-100. Upon addition of 0.01% hydrogen peroxide and 10 mM O-phenylenediamine (final concentrations) in Tris buffer, increase in absorbance is monitored for one minute at 450 nm. The presence of eosinophils in the uterus is an indication of estrogenic activity of a compound. The maximal velocity of a 15 second interval is determined over the initial, linear portion of the reaction curve.

Inhibition of Bone Loss (Osteoporosis) Test Procedure

Following the general preparation procedure described above, the rats are treated daily for thirty-five days (6 rats per treatment group) and sacrificed by carbon dioxide asphyxiation on the 36th day. The thirty-five day time period is sufficient to allow maximal reduction in bone density, measured as described herein. At the time of sacrifice, the uteri are removed, dissected free of extraneous tissue, and the fluid contents are expelled before determination of wet weight in order to confirm estrogen deficiency associated with complete ovariectomy. Uterine weight is routinely reduced about 75% in response to ovariectomy. The uteri are then placed in 10% neutral buffered formalin to allow for subsequent histological analysis.

The right femurs are excised and digitilized X-rays generated and analyzed by an image analysis program (NIH image) at the distal metaphysis. The proximal aspect of the tibiae from these animals are also scanned by quantitative computed tomography. In accordance with the above procedures, F-I or ethynyl estradiol (EE₂) in 20% hydroxypropyl β-cyclodextrin are orally administered to test animals.

Therapeutic Methods of Use and Dosages

Various diseases and conditions described to be treated herein, are well known and appreciated by those skilled in the art. It is also recognized that one skilled in the art may affect the associated diseases and conditions by treating a patient presently afflicted with the diseases or conditions or by prophylactically treating a patient afflicted with the diseases or conditions with a therapeutically effective amount of the compounds of formula (I).

As used herein, the term “patient” refers to a warm blooded animal such as a mammal that is afflicted with a particular estrogen receptor-beta mediated disease. It is understood that guinea pigs, dogs, cats, rats, mice, horses, cattle, sheep, and humans are examples of animals within the scope of the meaning of the term.

As used herein, the term “therapeutically effective amount” of a compound of formula (I) refers to an amount which is effective in controlling diseases and conditions associated with estrogen receptor-beta mediated diseases such as prostate cancer, benign prostatic hyperplasia, testicular cancer, cardiovascular diseases, neurodegenerative disorders, urinary incontinence, CNS disorders, GI tract disorders, and osteoporosis. The term “controlling” is intended to refer to all processes wherein there may be a slowing, interrupting, arresting, or stopping of the progression of the diseases and conditions described herein, but does not necessarily indicate a total elimination of all disease and condition symptoms, but does include prophylactic treatment of the diseases and conditions associated with estrogen receptor-beta mediated diseases such as prostate cancer, benign prostatic hyperplasia, testicular cancer, cardiovascular diseases, neurodegenerative disorders, urinary incontinence, CNS, GI tract disorders, and osteoporosis.

A therapeutically effective amount can be readily determined by the attending diagnostician, as one skilled in the art, by the use of conventional techniques and by observing results obtained under analogous circumstances. In determining the therapeutically effective amount, the dose, a number of factors are considered by the attending diagnostician, including, but not limited to: the species of mammal; its size, age, and general health; the specific disease involved; the degree of or involvement or the severity of the disease; the response of the individual patient; the particular compound administered; the mode of administration; the bioavailability characteristic of the preparation administered; the dose regimen selected; the use of concomitant medication; and other relevant circumstances.

A therapeutically effective amount of a compound of formula (I) is expected to vary from about 0.001 milligram per kilogram of body weight per day (mg/kg/day) to about 100 mg/kg/day. Preferred amounts can be determined by one skilled in the art.

In effecting treatment of a patient afflicted with the diseases and conditions described above, a compound of formula (I) can be administered in any form or mode which makes the compound bioavailable in a therapeutically effective amount, including oral, inhalation, and parenteral routes. For example, compounds of formula (I) can be administered orally, by inhalation of an aerosol or dry powder, subcutaneously, intramuscularly, intravenously, transdermally, intranasally, rectally, topically, and the like. Oral or inhalation administration is generally preferred for treatment of respiratory diseases, e.g. asthma. One skilled in the art of preparing formulations can readily select the proper form and mode of administration depending upon the particular characteristics of the compound selected, the disease or condition state to be treated, the stage of the disease or condition, and other relevant circumstances. (Remington's Pharmaceutical Sciences, 18th Edition, Mack Publishing Co. (1990)).

The compounds of the present invention can be administered alone or in the form of a pharmaceutical composition in combination with pharmaceutically acceptable carriers or excipients, the proportion and nature of which are determined by the solubility and chemical properties of the compound selected, the chosen route of administration, and standard pharmaceutical practice. The compounds of the present invention, while effective themselves, may be formulated and administered in the form of their pharmaceutically acceptable salts, such as acid addition salts or base addition salts, for purposes of stability, convenience of crystallization, increased solubility and the like.

In another embodiment, the present invention provides pharmaceutical compositions comprising a therapeutically effective amount of a compound of formula (I) in admixture or otherwise in association with one or more pharmaceutically acceptable carriers or excipients.

The pharmaceutical compositions are prepared in a manner well known in the pharmaceutical art. The carrier or excipient may be a solid, semi-solid, or liquid material, which can serve as a vehicle or medium for the active ingredient. Suitable carriers or excipients are well known in the art. The pharmaceutical composition may be adapted for oral, inhalation, parenteral, or topical use and may be administered to the patient in the form of tablets, capsules, aerosols, inhalants, suppositories, solution, suspensions, or the like.

The compounds of the present invention may be administered orally, for example, with an inert diluent or with an edible carrier. They may be enclosed in gelatin capsules or compressed into tablets. For the purpose of oral therapeutic administration, the compounds may be incorporated with excipients and used in the form of tablets, troches, capsules, elixirs, suspensions, syrups, wafers, chewing gums and the like. These preparations should contain at least 4% of the compound of the present invention, the active ingredient, but may be varied depending upon the particular form and may conveniently be between 4% to about 70% of the weight of the unit. The amount of the compound present in compositions is such that a suitable dosage will be obtained. Preferred compositions and preparations according to the present invention may be determined by someone skilled in the art.

The tablets, pills, capsules, troches and the like may also contain one or more of the following adjuvants: binders such as microcrystalline cellulose, gum tragacanth or gelatin; excipients such as starch or lactose, disintegrating agents such as alginic acid, Primogel, corn starch and the like; lubricants such as magnesium stearate or Sterotex; glidants such as colloidal silicon dioxide; and sweetening agents such as sucrose or saccharin may be added or a flavoring agent such as peppermint, methyl salicylate or orange flavoring. When the dosage unit form is a capsule, it may contain, in addition to materials of the above type, a liquid carrier such as polyethylene glycol or a fatty oil. Other dosage unit forms may contain other various materials that modify the physical form of the dosage unit, for example, as coatings. Thus, tablets or pills may be coated with sugar, shellac, or other enteric coating agents. A syrup may contain, in addition to the present compounds, sucrose as a sweetening agent and certain preservatives, dyes and colorings and flavors. Materials used in preparing these various compositions should be pharmaceutically pure and non-toxic in the amounts used.

For the purpose of parenteral therapeutic administration, the compounds of the present invention may be incorporated into a solution or suspension. These preparations should contain at least 0.1% of a compound of the inyention, but may be varied to be between 0.1 and about 50% of the weight thereof. The amount of the compound of formula (I) present in such compositions is such that a suitable dosage will be obtained. Preferred compositions and preparations are able to be determined by one skilled in the art.

The compounds of the present invention may also be administered by inhalation, such as by aerosol or dry powder. Delivery may be by a liquefied or compressed gas or by a suitable pump system that dispenses the compounds of the present invention or a formulation thereof. Formulations for administration by inhalation of compounds of formula (I) may be delivered in single phase, bi-phasic, or tri-phasic systems. A variety of systems are available for the administration by aerosols of the compounds of formula (I). Dry powder formulations are prepared by either pelletizing or milling the compound of formula (I) to a suitable particle size or by admixing the pelletized or milled compound of formula (I) with a suitable carrier material, such as lactose and the like. Delivery by inhalation includes the necessary container, activators, valves, subcontainers, and the like. Preferred aerosols and dry powder formulations for administration by inhalation are able to be determined by one skilled in the art.

The compounds of the present invention may also be administered topically, and when done so the carrier may suitably comprise a solution, ointment or gel base. The base, for example, may comprise one or more of the following: petrolatum, lanolin, polyethylene glycols, bee wax, mineral oil, diluents such as water and alcohol, and emulsifiers and stabilizers. Topical formulations may contain a concentration of the formula (I) or its pharmaceutical salt from about 0.1 to about 10% w/v (weight per unit volume).

The solutions or suspensions may also include one or more of the following adjuvants: sterile diluents such as water for injection, saline solution, fixed oils, polyethylene glycols, glycerin, propylene glycol or other synthetic solvents; antibacterial agents such as benzyl alcohol or methyl paraben; antioxidants such as ascorbic acid or sodium bisulfite; chelating agents such as ethylene diaminetetraacetic acid; buffers such as acetates, citrates or phosphates and agents for the adjustment of tonicity such as sodium chloride or dextrose. The parenteral preparation can be enclosed in ampules, disposable syringes or multiple dose vials made of glass or plastic.

What is claimed is:

1. A compound of formula (I) comprising: 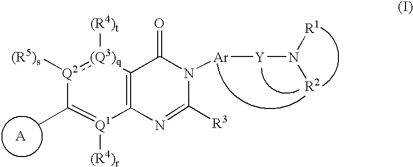
a pharmaceutically acceptable salt, solvate, or physiologically functional derivative thereof, wherein:
\textcircled{A} is aryl or heteroaryl, optionally substituted one to four times by at least one substituent selected from the group consisting of C₁₋₆ straight or branched alkyl, alkenyl, halo, amino, alkylamino, dialkylamino, hydroxy,
C₁₋₆ alkoxy, cyano, nitro, and alkylthio groups;
the dashed line connecting Q² to Q³ represents an optional bond;
q, r, s, and t are each independently 0 or 1;
when q is 1, the dashed line is a bond;
Q¹ and Q³ are each independently C or N;
when q is 0 then Q² is N, S, or O;
when q is 1, then Q² is C or N; when q is 1 and Q² is N, then s is 0;
when Q² is S or O, s is 0;
when Q¹ is N, r is 0;
when Q³is N, t is 0;
R³is selected from the group consisting of hydrogen, amino, C₁₋₆ straight or branched alkyl, C₃₋₆ cycloalkyl, and C₁₋₃ alkylthio;
when Q¹ or Q³ is C, then each corresponding R⁴ is independently selected from the group consisting of hydrogen, C₁₋₆ straight or branched alkyl, C₃₋₆ cycloalkyl, C₁₋₆ alkoxy, amino, alkylamino, dialkylamino, hydroxy, cyano, alkylthio, and halo;
when q is 1 and Q² is C or when q is 0 and Q² is N, then R⁵ is selected from the group consisting of hydrogen, C₁₋₆ straight or branched alkyl, C₃₋₆ cycloalkyl, C₁₋₆ alkoxy, amino, alkylamino, dialkylamino, hydroxy, cyano, alkylthio, and halo;
Ar is an optionally substituted fused bicyclic ring;
Y is a bond or a C₁₋₆ alkylene, optionally substituted;
(i) R¹ and R² are each independently selected from the group consisting of hydrogen, C₁₋₆ straight or branched alkyl, C₃₋₆ cycloalkyl, and a 5- or 6-membered heterocycle wherein said alkyl, said cycloalkyl, and said heterocycle are optionally substituted one to four times by at least one substituent selected from the group consisting of phenyl, C₁₋₃ alkyl, hydroxy, oxo, alkoxy and halo;
or (ii) R¹ and R² are each selected from the group consisting of aryl and a 5- or 6-membered heteroaryl containing 1, 2, or 3 heteroatoms selected from N, O, and S, wherein said aryl and said heteroaryl are optionally substituted 1, 2, or 3 times with at least one substituent selected from halo, C₁₋₆ straight or branched alkyl, C₃₋₆ cycloalkyl, C₁₋₆ alkenyl, C₃₋₆ cycloalkenyl, hydroxy, C₁₋₆ alkoxy, oxo, amino, C₁₋₆ alkylamino, C₁₋₆ dialkylamino, C₁₋₆ alkylthio, C₁₋₆ alkylsulfinyl, and phenyl;
or (iii) R¹ and R² together with the nitrogen atom to which they are bonded form a 4-8 membered heterocyclic ring or a 7-11 membered bicyclic heterocyclic ring, wherein said 4-8 membered heterocyclic ring and said 7-11 membered bicyclic heterocyclic ring contain 1, 2 or 3 heteroatoms selected from the group consisting of N, O, and S, and wherein either said heterocyclic ring or said bicyclic heterocyclic ring is optionally substituted one to four times by at least one substituent selected from the group consisting of by phenyl, C₁₋₃ alkyl, hydroxy, C₁₋₃ alkoxy, oxo, amino, C₁₋₆ alkylamino, C₁₋₆ dialkylamino, or halo;
or (iv) R² together with the adjacent nitrogen atom and Y may form an optionally substitued nitrogen-containing heterocycle, or R² together with the adjacent nitrogen atom, Y, and Ar may form an optionally substituted nitrogen-containing heterocycle or salt thereof, wherein said heterocycle is optionally substituted one to four times by at least one substituent selected from the group consisting of phenyl, C₁₋₃ alkyl, hydroxy, C₁₋₃ alkoxy, oxo, amino, C₁₋₆ alkylamino, C₁₋₆ dialkylamino, and halo.

2. The compound according to claim 1 wherein said
\textcircled{A} is an aryl substituted with at least one substituent selected from the group consisting of halo, C₁₋₃ alkyl, and C₁₋₃ alkoxy.

3. The compound according to claim 2 wherein \textcircled{A} is an aryl substituted with a group selected from the group consisting of fluoro, chloro, and methoxy.

4. The compound according to claim 1 wherein said \textcircled{A} is a halo-substituted aryl or a halo-substituted heteroaryl; q is 0; s is 0; Q¹ is C; Q² is S; and R⁴ is hydrogen or halo.

5. The compound according to claim 4 wherein \textcircled{A} is 4-chlorophenyl; and R³and R⁴are each hydrogen.

6. The compound according to claim 1 wherein Q¹, Q², and Q³ are each C; and q, r, s, and t are 1.

7. The compound according to claim 1 wherein Q¹ is N; Q² is S; and q, r, s, and t are 0.

8. The compound according to claim 1 wherein R³ is hydrogen or C₁₋₃ alkyl.

9. The compound according to claim 8 wherein R³ is hydrogen or methyl.

10. The compound according to claim 1 wherein Ar is a 9-14 membered fused polycyclic aromatic ring or a 9-14 membered fused polycyclic heteroaromatic ring.

11. The compound of claim 10 wherein the fused polycyclic aromatic ring or the fused polycyclic heteroaromatic ring is a ten-membered ring.

12. The compound of claim 11 wherein said fused polycyclic aromatic ring is naphthalene or the fused polycyclic heteroaromatic ring is quinoline.

13. The compound of claim 11 wherein Y is an optionally substituted C₁₋₆ alkylene.

14. The compound of claim 13 wherein Y is a C₁₋₃ alkylene, optionally substituted.

15. The compound of claim 14 wherein Y is methylene.

16. The compound according to claim 1 wherein in (i), R¹ and R² are each selected independently from the group consisting of hydrogen, C₁₋₆ straight or branched alkyl, and C₃₋₆ alkyl.

17. The compound according to claim 16 wherein in (i), R¹ and R² are selected independently from the group consisting of hydrogen, C₁₋₃ straight or branched alkyl, and C₃₋₆ alkyl.

18. The compound according to claim 1 wherein, in (ii), either R¹ or R² is aryl or heteroaryl, the other remaining R¹ or R² is hydrogen, C₁₋₆ alkyl, or a C₃₋₆ cycloalkyl.

19. The compound according to claim 1 wherein, in (iii), R¹ and R² together with the nitrogen atom to which they are bonded form a 5- or 6-membered heterocyclic ring or an 8- to 11-membered bicyclic heterocyclic ring; having 1 or 2 heteroatoms selected from the group N, O, and S; wherein said heterocyclic ring and said bicyclic heterocyclic ring are optionally substituted up to two times with a substituent selected from the group consisting of oxo and halo.

20. The compound according to claim 19 wherein R¹ and R² together with the nitrogen atom to which they are bonded form a heterocyclic ring selected from the group consisting of morpholine, piperidine, piperazine, pyrrolidine, 1,3-thiazolidine, 1H-imidazole, 4,5-dihydro-1H-imidazole, 2,3-dihydroindole, 1,2,3,4-tetrahydroquinoline, and 1,2,3,4-tetrahydroisoquinoline; and wherein said heterocyclic ring is optionally substituted one to four times by at least one substituent selected from the group consisting of phenyl, C₁₋₃ alkyl, hydroxy, alkoxy, oxo, and halo.

21. The compound according to claim 1 wherein, in (iv), Y is a C₁₋₆ alkylene and R² is linked to said Y to form a 3 to 7-membered ring.

22. The compound according to claim 21 wherein said ring is a 5 to 7-membered ring optionally substituted one to four times by at least one substituent selected from the group consisting of phenyl, C₁₋₃ alkyl, hydroxy, alkoxy, oxo, and halo.

23. The compound according claim 1 wherein the compound is selected from the group consisting of
6-(4-chlorophenyl)-3-{6-[(4-hydroxy-1-piperidinyl)methyl]-2-naphthalenyl}thieno[3,2-d]pyrimidin-4(3H)-one;
6-(4-chlorophenyl)-3-[6-(pyrrolidin-1-ylmethyl)-2-naphthyl]thieno[3,2-d]pyrimidin-4(3H)-one;
6-(4-chlorophenyl)-3-{2-[(4-methylpiperazin-1-yl)methyl]-1-benzothien-5-yl}thieno[3,2-d]pyrimidin-4(3H)-one;
6-(4-fluorophenyl)-3-[2-(pyrrolidin-1-ylmethyl)quinolin-6-yl]thieno[3,2-d]pyrimidin-4(3H)-one;
6-(4-fluorophenyl)-3-[2-(piperidin-1-ylmethyl)quinolin-6-yl]thieno[3,2-d]pyrimidin-4(3H)-one;
6-(4-chlorophenyl)-3-{2-[(2-methyl-4,5-dihydro-1H-imidazol-1-yl)methyl]quinolin-6-yl}thieno[3,2-d]pyrimidin-4(3H)-one;
6-(4-chlorophenyl)-3-{2-[(2,2,6,6-tetramethylpiperidin-1-yl)methyl]quinolin-6-yl}thieno[3,2-d]pyrimidin-4(3H)-one;
and 6-phenyl-3-[2-(pyrrolidin-1-ylmethyl)quinolin-6-yl]thieno[3,2-d]pyrimidin-4(3H)-one.

24. The compound of claim 10 wherein Ar is a 9-membered fused polycyclic heteroaromatic ring.

25. The compound of claim 24 wherein Ar is benzimidazole, indole, benzothiophene, benzothiazole, or benzofuran.

26. The compound of claim 25 wherein Y is a bond or C₁₋₃ alkylene.

27. The compound of claim 26 wherein Y is a bond or methylene.

28. The compound according to claim 24 wherein, in (i), R¹ and R² are each selected independently from the group consisting of hydrogen, C₁₋₆ straight or branched alkyl, and C³⁻⁶ alkyl.

29. The compound according to claim 28 wherein, in (i), R¹ and R² each are selected independently from the group consisting of hydrogen, C₁₋₃ straight or branched alkyl, and C₃₋₆ alkyl.

30. The compound according to claim 24 wherein, in (ii), either R¹ or R² is aryl or heteroaryl, the other remaining R¹ or R² is hydrogen, C₁₋₆ alkyl, or a C₃₋₆ cycloalkyl.

31. The compound according to claim 24 wherein, in (iii), R¹ and R² together with the nitrogen atom to which they are bonded form a 5- or 6-membered heterocyclic ring or an 8- to 11-membered bicyclic heterocyclic ring having 1 or 2 heteroatoms selected from the group N, O, and S; and wherein said heterocyclic ring and said bicyclic heterocyclic ring may be optionally substituted up to two times with a substituent selected from the group consisting of oxo and halo.

32. The compound according to claim 31 wherein said ring is selected from the group consisting of morpholine, piperidine, piperazine, pyrrolidine, 1,3-thiazolidine, 1H-imidazole, 4,5-dihydro-1H-imidazole, 2,3-dihydroindole, 1,2,3,4-tetrahydroquinoline, or 1,2,3,4-tetrahydroisoquinoline; and wherein said heterocyclic ring is optionally substituted one to four times by at least one substituent selected from the group consisting of phenyl, C₁₋₃ alkyl, hydroxy, alkoxy, oxo, and halo.

33. The compound according to claim 24 wherein, in (iv), Y is a C₁₋₆ alkylene and is linked to R² to form a 3-7 membered ring.

34. The compound according to claim 33 wherein said 3-7 membered ring is a 5 to 7 membered ring optionally substituted one to four times by at least one substitutent selected from the group consisting of phenyl, C₁₋₃ alkyl, hydroxy, alkoxy, oxo, and halo.

35. The compound according claim 24 wherein the compound is
6-(4-chlorophenyl)-3-[2-(dimethylamino)-1-methyl-1H-benzimidazol-6-yl]thieno[3,2-d]pyrimidin-4(3H)-one.

36. A process for preparing a compound of claim 1 comprising reacting an aniline of formula (II) 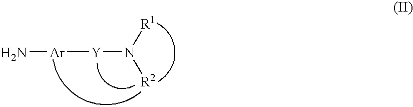
with a compound of formula (III) 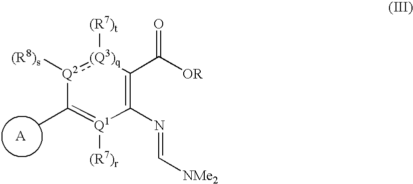
while heating in a solvent; wherein \textcircled{A}, R⁵, R⁴, R³, R², R¹, Ar, Y, Q¹, Q², Q³, q, r, s, and t, are as defined in formula (I); and R is C₁-C₄ alkyl.

37. A process for preparing a compound of claim 1 comprising coupling an amino acid of formula (IV) 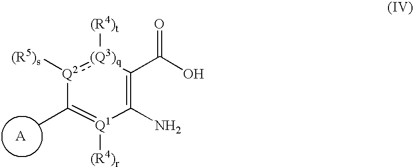
with an aniline of formula (II) 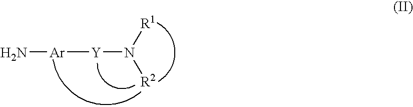
in a solvent in the presence of at least one coupling agent to produce a compound of formula (V) 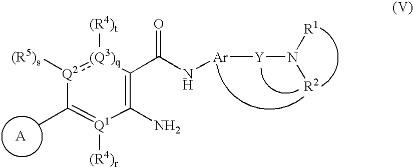
and cyclizing said compound of formula (V) to form a compound of formula (I) and
wherein \textcircled{A}, R⁵, R⁴, R³, R², R¹, Ar, Y, Q¹, Q², Q³, q, r, s, and t, are as defined in formula (I).

38. A process for preparing a compound of claim 1 comprising reaction of a compound of formula (Va) 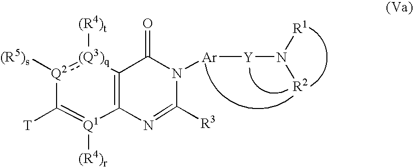
with a boronic acid and a palladium catalyst using a Suzuki coupling reaction or with an organostannane reagent and a palladium catalyst using a Stille coupling reaction
and wherein \textcircled{A}, R⁵, R⁴, R³, R², R¹, Ar, Y, Q¹, Q², Q³, q, r, s, and t, are as defined in formula (I) and T is a leaving group.

39. A process for preparing a compound of claim 1 comprising coupling an amino ester of formula (III) 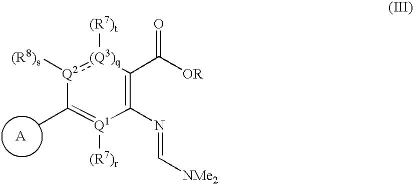
with an aniline of formula (II) 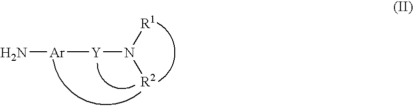
in a solvent in the presence of trimethylaluminum to produce a compound of formula (Vb) 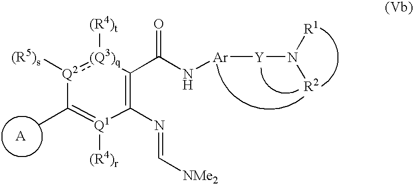
and cyclizing said compound of formula (Vb) to form a compound of formula (I) and
wherein wherein \textcircled{A}, R⁵, R⁴, R³, R², R¹, Ar, Y, Q¹, Q², Q³, q, r, s, and t, are as defined in formula (I).

40. A process for preparing a compound of claim 1 wherein R⁵ is hydrogen comprising reacting a sulfur-containing compound of formula (VI) 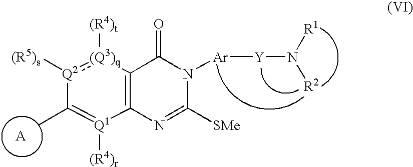
with a Raney nickel reductant in the presence of a solvent and wherein wherein \textcircled{A}, R⁵, R⁴, R³, R², R¹, Q¹, Q², Q³, q, r, s, and t, are as defined in formula (I).

41. A process for preparing a compound of claim 1 comprising the alkylation of an amine of formula (VII)
H—NR¹R² (VII)
with an alkylating agent of formula (VIII) 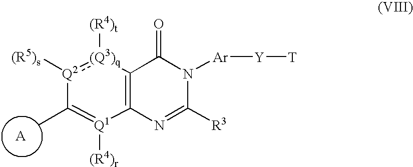
wherein T is a leaving group, and wherein \textcircled{A}, R⁵, R⁴, R³, R², R¹, Ar, Y, Q¹, Q², Q³, q, r, s, and t, are as defined in formula (I).

42. A process for preparing a compound of claim 1 comprising the treatment of an amine of formula (VII)
H—NR¹R² (VII)
with a strong base such as sodium hexamethyldisilazane and reaction with an ester of formula (III) 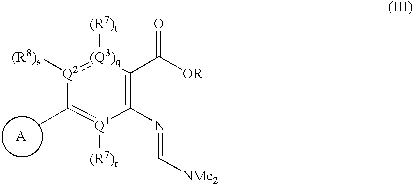
in a solvent such as tetrahydrofuran to produce a compound of formula (Vb) 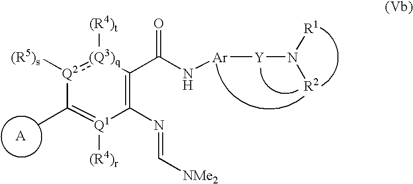
and cyclizing said compound of formula (Vb) to form a compound of formula (I) and
wherein wherein \textcircled{A}, R⁵, R⁴, R³, R², R¹, Ar, Y, Q¹, Q², Q³, q, r, s, and t, are as defined in formula (I).

43. A method of treating obesity, diabetes, depression, or anxiety in a mammal comprising the administration to said mammal of an effective amount of a compound according to claim 1 or a pharmaceutically acceptable salt, solvate, or physiologically functional derivative thereof.

44. The method of claim 43 wherein said mammal is a human.

45. A method of treating obesity, diabetes, depression, or anxiety in a mammal comprising the administration of an effective amount of a pharmaceutical composition containing a compound according to claim 1, a pharmaceutically acceptable salt, solvate, or physiologically functional derivative thereof to said mammal.

46. The method of claim 45 wherein said mammal is a human.

47. The compound of claim 1, a salt, a solvate, or physiologically functional derivative thereof in combination with at least one species selected from the group consisting of an agent for treating diabetes, an agent for treating hypertension, and an agent for treating arteriosclerosis.

48. The compound of claim 1, a salt, a solvate, or a physiologically functional derivative thereof in combination with at least one species for the treatment of obesity selected from the group consisting of (i) human ciliary neurotrophic factor, (ii) a CB-1 antagonist or inverse agonist, (iii) a neurotransmitter reuptake inhibitor, (iv) a lipase inhibitor, (v) an MC4R agonist, (vi) a 5-HT2c agonist, and (vii) a ghrelin receptor agonist or antagonist.

49. A pharmaceutical composition comprising a compound of claim 1 and at least one excipient or carrier.

50. (canceled)
